(19) United States
(12) Patent Application Publication
(10) Pub. No.: US 2024/0200065 A1
(43) Pub. Date: Jun. 20, 2024

(54) COMPOSITIONS AND METHODS USEFUL FOR THE TREATMENT OF H-ABC LEUKODYSTROPHY

(71) Applicant: THE CHILDREN'S HOSPITAL OF PHILADELPHIA, Philadelphia, PA (US)

(72) Inventors: Adeline VANDERVER, Bryn Mawr, PA (US); Sunetra SASE, Philadelphia, PA (US); Akshata ALMAD, Philadelphia, PA (US)

(73) Assignee: THE CHILDREN'S HOSPITAL OF PHILADELPHIA, Philadelphia, PA (US)

(21) Appl. No.: 18/287,150

(22) PCT Filed: Apr. 18, 2022
(86) PCT No.: PCT/US22/25246

§ 371 Date: Oct. 16, 2023

Related U.S. Application Data
(60) Provisional application No. 63/175,988, filed on Apr. 16, 2021.

(51) Int. Cl. C12N 15/113; A61K 31/7125; A61P 25/14

(52) U.S. Cl. CPC C12N 15/113; A61K 31/7125; A61P 25/14

(57) ABSTRACT

Compositions and methods for the treatment of H-ABC leukodystrophy are disclosed.

### CROSS-REFERENCE TO RELATED APPLICATIONS

This application claims priority to U.S. Provisional Application No. 63/175,998 filed Apr. 16, 2021, which is incorporated herein by reference as though set forth in full.

### INCORPORATION-BY-REFERENCE OF MATERIAL SUBMITTED IN ELECTRONIC FORM

Incorporated herein by reference in its entirety is the Sequence Listing submitted via EFS-Web as a text file named SEQLIST.txt, created Apr. 18, 2022 and having a size of 77,824 bytes

### FIELD OF THE INVENTION

This invention relates the fields of H-ABC leukodystrophy and methods of use of small molecule compositions, particularly modified antisense molecules, for ameliorating symptoms of the same. More specifically, the invention provides modified nucleic acid-based therapeutics which down modulate tubular alpha 4A (TUBB4A), thereby improving dystonia, ataxia, altered gait and motor dysfunction in patients in need thereof.

### BACKGROUND OF THE INVENTION

Several publications and patent documents are cited throughout the specification in order to describe the state of the art to which this invention pertains. Each of these citations is incorporated herein by reference as though set forth in full.

Hypomyelination with atrophy of the basal ganglia and cerebellum, also called H-ABC, is a rare genetic disorder that progressively damages the nervous system specifically targeting two parts of the brain: the basal ganglia and the cerebellum, which control the body's actions and movement (Hersheson et al., 2013; Simons et al., 2013). H-ABC is a type of leukodystrophy, a group of conditions that affect the white matter of the brain. As used herein, H-ABC is intended to refer also to a larger group of related disorders, e.g., TUBB4A associated disorders, with variable manifestations of dystonia, hypomyelination, and developmental abnormalities (Blumkin et al., 2014; Ferreira et al., 2014; Pizzino et al., 2014). These diseases are associated with impairment of the myelination process resulting in damage to the myelin sheath, which surrounds and protects the nerve cells in the brain and spinal cord and speeds transmission of messages between cells. Typically, myelination occurs during the first few years of life. In H-ABC, there is hypomyelination due to the body's inability to produce myelin at normal levels, thereby preventing or otherwise impeding, normal myelination of the brain. In certain cases, the nerves outside the brain may be affected. Unfortunately, the condition also reduces the size and function of the basal ganglia and the cerebellum. As a result, people with H-ABC often have motor problems, including stiffness of the muscles and joints, low muscle tone, difficulty with controlling movements, and problems with balance and coordination (Blumkin et al., 2014; Ferreira et al., 2014; Pizzino et al., 2014; Simons et al., 2013).

H-ABC is caused by a mutation in the TUBB4A gene which often presents as a random mutation in the person who develops the condition. In these cases, neither parent is a carrier, and the chances of having another child with the disease is extremely low. There is a small chance that one parent may carry the mutation in some cells (mosaicism), which means that they would not have symptoms but still be able to transmit the disorder to other children.

Currently, there is no known cure for this disease. New therapeutic agents for ameliorating and improving H-ABC symptoms are urgently needed.

### SUMMARY OF THE INVENTION

In accordance with the present invention, a method of lowering TUBB4A levels in targeted cells, thereby improving symptoms of H-ABC leukodystrophy in a patient in need thereof is provided. An exemplary method entails administration of a therapeutically effective amount of a composition comprising a synthetic inhibitory nucleic acid molecule targeting TUBB4A, wherein lowering TUBB4A levels reduces one or more of i) delayed motor development; ii) cognitive dysfunction; iii) gait dysfunction; iv) ataxia; v) intention tremor; vi) dysarthria; vii) dysphonia; and viii) aberrant hypomyelination; in said patient. In preferred embodiments the synthetic nucleic acid molecule is selected from an antisense oligonucleotide, an shRNA, an siRNA, and a guide strand suitable for CRISPR editing TUBB4A targeted nucleic acids. Also provided is a method of treating, delaying the onset of, ameliorating, and/or reducing a disease, disorder and/or condition, or a symptom thereof, associated with H-ABC leukodystrophy in a patient in need thereof. In one aspect the method comprises administering to the patient a therapeutically effective amount of a synthetic inhibitory nucleic acid targeting TUBB4A, wherein the disease, disorder and/or condition, or the symptom thereof, associated with H-ABC leukodystrophy is treated, inhibited, the onset delayed, ameliorated, and/or reduced in the patient. As above, the synthetic nucleic acid molecule is selected from an antisense oligonucleotide, an shRNA, an siRNA, and a guide strand suitable for CRISPR editing. In certain embodiments, the synthetic inhibitory nucleic acid targeting TUBB4A is listed in Table 3 or Table 5 and is modified to increase stability and/or uptake in vivo. In certain embodiments, the synthetic nucleic acid encoding said antisense oligonucleotide is cloned into a vector. In other embodiments, the leukodystrophy is a disease, disorder and/or condition is associated with hypomyelination. In other embodiments, the modified oligonucleotide comprises at least one modified internucleoside linkage, at least one nucleoside of the modified oligonucleotide comprising a modified sugar or at least one nucleoside of the modified oligonucleotide comprising a modified nucleobase. In another aspect of the invention, the method further comprises administration of an additional active pharmaceutical agent useful for treatment of leukodystrophy.

Also provided is a composition for reducing expression of TUBB4A, comprising a synthetic inhibitory nucleic acid molecule targeting, and specifically hybridizing to, a TUBB4A encoding nucleic acid, selected from an antisense oligonucleotide, an shRNA, an siRNA, and a guide strand suitable for CRISPR editing in a biologically acceptable carrier. In certain embodiments, the synthetic inhibitory nucleic acid is modified to increase stability in bodily fluids and/or uptake in a cell of interest. In preferred embodiments, the synthetic inhibitory nucleic acid is listed in Table 3 or in Table 5. The nucleic acid is optionally cloned into a vector. The vector may be a plasmid vector, an AAV vector and adenovirus associated vector. In certain embodiments, the oligo nucleotide is administered in a lipid nanoparticle complex. The synthetic inhibitory nucleic acid and, or vector or carrier comprising the same, may be present in a pharmaceutically acceptable carrier which is formulated via administration routes including, but not limited to ex vivo cellular administration, intracranial administration, parenteral administration, intramuscular, and intravenous administration. In a preferred embodiment, the composition is formulated for single intracerebroventricular (ICV) bolus injection.

Also provided is a kit comprising components suitable for practicing any of the aforementioned methods.

### BRIEF DESCRIPTION OF THE DRAWINGS

FIG. 1A-1D: The library of ASOs was screened in human A549N cells using electroporation with three replicates. After ASO electroporation at different concentrations (0.1 μM, 0.5 μM, 1 μM and 5 μM), RNA was extracted and subjected to qPCR to assess TUBB4A expression levels. TUBB4A expression is normalized to GAPDH (n=3, ***p<0.0001). FIG. 1A shows TUBB4A mRNA expression of non targeted control (NTC) (Cells treated with scrambled oligo) and ASO H1-H5. FIG. 1B shows TUBB4A mRNA expression of ASO H6-H12. FIG. 1C shows TUBB4A mRNA expression of ASO H13-H19. FIG. 1D shows TUBB4A mRNA expression of NTC, ASO H2A, ASO H2B, ASO H10A, ASO H10B, ASO HI 1A, ASO H11B, ASO H15A, and ASO H15B.

FIG. 2A-2E: The library of ASOs was screened in mouse HT-22 cell line using electroporation with three replicates. After ASO electroporation at different concentrations (0.1 μM, 0.5 μM, 1 μM and 5 μM), RNA was extracted and subjected to qPCR to assess the TUBB4A expression. TUBB4A expression is normalized to sfrs9 (n=3, ***p<0.0001). FIG. 2A shows Tubb4a mRNA expression of ASO 1-7. FIG. 2B shows TUBB4A mRNA expression of ASO 8-14. FIG. 2C shows TUBB4A mRNA expression of ASO H15-H21. FIG. 2D shows TUBB4A mRNA expression of ASO 4A, ASO 4B, ASO 6A, ASO 6B, ASO 7A, and ASO 7B. FIG. 2E shows TUBB4A mRNA expression of ASO 22-24 and NTC ASO1-ASO3.

FIG. 3A-3D: Efficient ASO-mediated TUBB4A transcript suppression RT-qPCR data showing the levels of TUBB4A transcript in the cerebral cortex, cerebellum, striatum from wild-type mice injected with ASO 7A (FIG. 3A), ASO 7B (FIG. 3B), ASO 6A (FIG. 3C) and ASO 6B (FIG. 3D), 10 days post-injection.

FIG. 4: SHYSY5Y cells were plated at 250,000 cells/well and treated with 10 μm of Biospring and Microsynth ASO for 96 hours. RNA was extracted to conduct qRT-PCR for testing the levels of TUBB4A.

FIG. 5: SHYSY5Y cells were plated at 250,000 cells/well and treated with 10 μm of Biospring and Microsynth ASO for 96 hours. RNA was extracted to conduct Biospring for testing the levels of TUBB4A.

FIG. 6A-6C: SHYSY5Y cells were plated at 10000 cells/well and treated with 10 μm of Biospring ASO for 24 hours. The viability (FIG. 6A), cytotoxicity (FIG. 6B), and apoptosis (FIG. 6C) were tested using Digitonin and Staurosporine as positive controls.

FIG. 7A-7C: SHYSY5Y cells were plated at 10000 cells/well and treated with 10 μm of Biospring ASOs for 48, 72 and 96 hours. The viability (FIG. 7A), cytotoxicity (FIG. 7B), and apoptosis (FIG. 7C) were tested using Digitonin and Staurosporine as positive controls.

FIG. 8: Selection of potential ASO candidates on control human iPSC derived neurons using Gymnosis. Human iPSC derived medium neurons were treated at day 37 and resupplemented after four (4) days with total one week treatment. RNA was extracted and TUBB4A expression was determined using qRT-PCR.

FIG. 9A-9C: ASO toxicity. Human iPSC derived medium neurons were treated at day 37 and resupplemented after four (4) days with total one week treatment. To evaluate potential toxicity of ASOs, RNA was extracted and BAX (FIG. 9A) and BCL-2 (FIG. 9B) expression was determined using qRT-PCR. Ratios of BAXBCL-2>1 indicates more apoptosis, whereas ratios <1 indicates less apoptosis (FIG. 9C).

FIG. 10: Dose response of potential ASO candidate on control human iPSC derived neurons using Gymnosis. Human iPSC derived medium neurons were treated at day and resupplemented after four (4) days with total one week treatment. RNA was extracted and TUBB4A expression was determined using qRT-PCR.

FIG. 11A-11C: ASO toxicity. Human iPSC derived medium neurons were treated at day 37 and resupplemented after four (4) days with total one week treatment. To evaluate potential toxicity of ASOs, RNA was extracted and BAX (FIG. 11A) and BCL-2 (FIG. 111B) expression was determined using qRT-PCR. Ratios of BAX/BCL-2>1 indicates more apoptosis, whereas ratios <1 indicates less apoptosis (FIG. 11C).

FIG. 12: ASO candidate H15 in aTris-EDTA buffer was used to assess the downregulation in mutant human iPSC derived neurons (TUBB4A^{D249N}) using Gymnosis. The wild-type medium spiny neurons (MSNs) were re-run to confirm the previous downregulation results: Human iPSC derived medium neurons were treated at d37 and re-supplemented on d4 with total one week treatment. RNA was extracted and TUBB4A expression was determined using qRT-PCR.

FIG. 13A-13C: ASO toxicity in iPSC derived human mutant MSNs. Human iPSC derived medium spiny neurons were treated at day 37 and resupplemented after four (4) days with total one week treatment. To evaluate potential toxicity of ASOs, RNA was extracted and BAX (FIG. 13A) and BCL-2 (FIG. 13B) expression was determined using qRT-PCR. Ratios of BAX/BCL-2 >1 indicates more apoptosis, whereas ratios <1 indicates less (FIG. 13C).

FIG. 14A-14B: Cortical mouse primary neurons were plated at 200K cells/well and treated with 1 and 5 μm of Biospring ASOs for 1 week. RNA was extracted to conduct qRT-PCR for testing the levels of TUBB4A. FIG. 14A shows TUBB4A ASO downregulation of ASOs 6-1 to ASO 7-4. FIG. 14B shows TUBB4A ASO downregulation of ASO 8-1 to ASO 18-3.

FIG. 15: Cortical mouse primary neurons were plated at 150K cells/well and treated with 1 and 5 μm of Biospring ASOs for 1 week. RNA was extracted to conduct qRT-PCR for testing the levels of TUBB4A.

FIG. 16A-16B: Mouse cortical neurons were plated at 20000 cells/well and treated with 5 μm of Biospring ASOs for 96 hours. The viability (FIG. 16A) and apoptosis (FIG. 16B) were tested using Digitonin and Staurosporine as positive controls.

FIG. 17A-17C: Motor behavior rotarod (FIG. 17A) and grip strength (FIG. 17B and FIG. 17C) was performed 30 days post ASO ICV injection in wild-type mice to assess the motor outcomes after ASO injection.

FIG. 18A-18B: ASO 7-2 (FIG. 18A) and 7-3 (FIG. 18B) were ICV injected at age of P60 (adult) and tissue was collected after 23-30 days. RNA was extracted and TUBB4A downregulation was determined using qRT-PCR.

FIG. 19A-19B: ASO 8-2 (FIG. 19A) and 8-3 (FIG. 19B) were ICV injected at age of P60 (adult) and tissue was collected after 23-30 days, RNA was extracted and TUBB4A downregulation was determined using qRT-PCR.

FIG. 20A-20C: ASO 18-1 (FIG. 20A) and 18-3 (FIG. 20B) were ICV injected at age of P60 (adult) and tissue was collected after 23-30 days, RNA was extracted and TUBB4A downregulation was determined using qRT-PCR. Weights of the mice after injection at P90, P97, and P104 are shown in FIG. 20C.

### DETAILED DESCRIPTION OF THE INVENTION

Hypomyelination and atrophy of basal ganglia and cerebellum (H-ABC) is a rare hypomyelinating leukodystrophy associated with causal variants in tubulin alpha 4 (TUBB4A). For example, p.Asp249Asn (D249N) is a recurring variant occurring in the majority of affected individuals. Monoallelic mutations in TUBB4A may also result in a larger spectrum of neurologic disorders ranging from an early onset encephalopathy to an adult-onset Dystonia type 4 (whispering dysphonia) (Blumkin et al., 2014; Ferreira et al., 2014; Pizzino et al., 2014; Simons et al., 2013). H-ABC is within this spectrum, and typically begins in early childhood characterized by dystonia, ataxia, altered gait and progressive motor dysfunction with loss of ambulation before the end of the first decade of life. To date, there is no therapeutic approach available for this progressive and disabling pediatric disorder. To understand how TUBB4A mutations cause H-ABC and to facilitate the development and pre-clinical testing of therapeutic strategies, our group has developed a knock-in mouse model harboring heterozygous (TUBB4A^{D249N}) or homozygous (TUBB4A^{D249N/D249N}) TUBB4A mutations using a CRIISPR-Cas9 approach.

In earlier studies we describe a mouse model of classical H-ABC (TUBB4A^{D249N/D249N}) which displays decreased survival, progressive motor dysfunction with tremor, abnormal gait and ataxia, thus recapitulating the phenotypic features of the disease. Neuropathological assessment of TUBB4A^{D249N/D249N} mice using immunolabeling and western blot on post-natal day (P) 14, P21 and end-stage P40 shows initial delay of myelination followed by ultimate demyelination. There is decrease of myelin proteins over time and dramatic loss of aspartocyclase activity (ASPA) positive oligodendrocytes (myelinating cells in CNS). Ultrathin brain sections for electron microscopy further demonstrated hypomyelination and ongoing loss of myelin in spinal cord and optic nerves of these mice. In addition, in vitro studies on oligodendrocytes in culture from TUBB4A^{D249N/D249N} mice demonstrated decreased maturation and myelin markers. Similarly, neuropathology demonstrates severe neuronal loss in the striatum and cerebellar granule cells. Further, effects in neurons in culture were noted with decreased neuronal survival along with unstable microtubule dynamics in cells from TUBB4A^{D249N/D249N} mice. TUBB4A^{D249N/D249N} mice provide a novel mouse model for H-ABC, and demonstrate the complexity of cellular physiology in this disorder, with potential microtubule instability from TUBB4A mutations, resulting in cell autonomous effects on oligodendrocytes, striatal neurons and cerebellar granule cells, and profound neurodevelopmental phenotypes.

We have also developed new therapeutic antisense oligonucleotides which effectively down modulate TUBB4A expression, thereby providing a new approach for treatment of leukodystrophy.

### Definitions

The present subject matter may be understood more readily by reference to the following detailed description which forms a part of this disclosure. It is to be understood that this invention is not limited to the specific products, methods, conditions or parameters described and/or shown herein, and that the terminology used herein is for the purpose of describing particular embodiments by way of example only and is not intended to be limiting of the claimed invention.

Unless otherwise defined herein, scientific and technical terms used in connection with the present application shall have the meanings that are commonly understood by those of ordinary skill in the art. Further, unless otherwise required by context, singular terms shall include pluralities and plural terms shall include the singular.

As employed above and throughout the disclosure, the following terms and abbreviations, unless otherwise indicated, shall be understood to have the following meanings.

In the present disclosure the singular forms “a,” “an,” and “the” include the plural reference, and reference to a particular numerical value includes at least that particular value, unless the context clearly indicates otherwise. Thus, for example, a reference to “a compound” is a reference to one or more of such compounds and equivalents thereof known to those skilled in the art, and so forth. The term “plurality”, as used herein, means more than one. When a range of values is expressed, another embodiment includes from the one particular and/or to the other particular value. Similarly, when values are expressed as approximations, by use of the antecedent “about,” it is understood that the particular value forms another embodiment. All ranges are inclusive and combinable.

The terms “comprising, having, or including” indicate that the claim is open and can read on components not recited.

The phrase “consisting essentially of” when referring to a particular nucleotide or amino acid means a sequence having the properties of a given SEQ ID NO. For example, when used in reference to an amino acid sequence, the phrase includes the sequence per se and molecular modifications that would not affect the functional and novel characteristics of the sequence.

The phrase “consisting of” indicates that the claim reads on only the components recited.

As used herein, the terms “component,” “composition,” “composition of compounds,” “compound,” “drug,” “pharmacologically active agent,” “active agent,” “therapeutic,” “therapy,” “treatment,” or “medicament” are used interchangeably herein to refer to a compound or compounds or composition of matter which, when administered to a subject (human or animal) induces a desired pharmacological and/or physiologic effect by local and/or systemic action. The terms “agent” and “test compound” denote a chemical compound, a mixture of chemical compounds, a biological macromolecule, or an extract made from biological materials such as bacteria, plants, fungi, or animal (particularly mammalian) cells or tissues. Biological macromolecules include siRNA, shRNA, antisense oligonucleotides, peptides, peptide/DNA complexes, and any nucleic acid based molecule which exhibits the capacity to modulate the activity of the TUBB4A containing nucleic acids described herein or their encoded proteins.

As used herein, “TUBB4A” refers to a gene which encodes a member of the beta tubulin family. Beta tubulins are one of two core protein families (alpha and beta tubulins) that heterodimerize and assemble to form microtubules. Mutations in this gene cause hypomyelinating leukodystrophy-6 and autosomal dominant torsion dystonia-4 and H-ABC, now more commonly referred to as TUBB4A-Associated Leukoencephalopathy. Reference sequences for TUBB4A include for example NM_001289123.1, NM_001289127.1 NM_001289129.1 which can be found on GenBank. Alternate splicing results in multiple transcript variants encoding different isoforms. The wild type TUBB4A protein sequence is found on UniProt, accession no. P04350-TBB4A_human. Several TUBB4A variants known to be associated with human disease have been identified and are listed below in Table 1. The present invention focuses on the D249N variant, however the findings are generalizable to other existing TUBB4A mutations.

TABLE 1
TUBB4A Variants

| | | | |
|---|---|---|---|
| c.76A > T | c.1190G > T | c.535G > A | c.731G > C |
| c.1052C > T | c.1228G > A | c.535G > C | c.731G > T |
| c.1054G > A | c.1254G > T | c.535G > T | c.743C > A |
| c.1061G > A | c.1325G > A | c.538G > A | c.745G > A |
| c.1062C > G | c.286G > A | c.539A > C | c.755A > G |
| c.1088T > C | c.293G > A | c.568C > T | c.763G > A |
| c.1099T > A | c.395G > C | c.5G > A | c.785G > A |
| c.1099T > C | c.467G > T | c.691G > A | c.796T > A |
| c.1162A > G | c.4C > G | c.716G > A | c.811G > A |
| c.1164G > A | c.4C > T | c.716G > T | c.845G > C |
| c.1164G > T | c.518A > T | c.730G > A | c.874C > A |
| c.1171C > T | c.523G > A | c.730G > C | c.916G > A |
| c.1172G > A | c.533C > A | c.730G > T | c.968T > G |
| c.1172G > T | c.533C > G | c.731G > A | c.971A > C |
| c.1181T > G | c.533C > T | | |

Table 2 provides a listing of certain amino acid changes associated with different forms of TUBB4A-Associated Leukoencephalopathy.

TABLE 2

| | Human | Cell Type |
|---|---|---|
| Mutation | Phenotype | Affected |
| p.Arg2Gly | Whispering | Neurons |
| R2G | Dysphonia | |
| p.Asp249Arg | Classical | Neurons and |
| (D249N) | H-ABC | Oligodendrocytes |
| p.Arg28Pro | Isolated | Oligodendrocytes |
| R282P | hypomyelination | |
| p.Arg391His | Isolated | Oligodendrocytes |
| (R391H) | hypomyelination | |

As used herein, the terms “treatment” or “therapy” (as well as different forms thereof) include preventative (e.g., prophylactic), curative or palliative treatment. As used herein, the term “treating” includes alleviating or reducing at least one adverse or negative effect or symptom of a condition, disease or disorder.

The terms “subject,” “individual,” and “patient” are used interchangeably herein, and refer to an animal, for example a human, to whom treatment, including prophylactic treatment, with the pharmaceutical composition according to the present invention, is provided. The term “subject” as used herein refers to human and non-human animals. The terms “non-human animals” and “non-human mammals” are used interchangeably herein and include all vertebrates, e.g., mammals, such as non-human primates, (particularly higher primates), sheep, dog, rodent, (e.g. mouse or rat), guinea pig, goat, pig, cat, rabbits, cows, horses and non-mammals such as reptiles, amphibians, chickens, and turkeys.

The terms “polynucleotide”, “nucleotide”, “nucleotide sequence”, “nucleic acid” and “oligonucleotide” are used interchangeably. They refer to a polymeric form of nucleotides of any length, either deoxyribonucleotides or ribonucleotides, or analogs thereof. A polynucleotide may comprise one or more modified nucleotides, such as methylated nucleotides and nucleotide analogs. If present, modifications to the nucleotide structure may be imparted before or after assembly of the polymer. The sequence of nucleotides may be interrupted by non-nucleotide components. A polynucleotide may be further modified after polymerization, such as by conjugation with a labeling component.

As used herein the term “wild type” is a term of the art understood by skilled persons and means the typical form of an organism, strain, gene or characteristic as it occurs in nature as distinguished from mutant or variant forms. As used herein the term “variant” should be taken to mean the exhibition of qualities that have a pattern that deviates from the wild type or a comprises non naturally occurring components.

The terms “non-naturally occurring” or “engineered” are used interchangeably and indicate the involvement of the hand of man. The terms, when referring to nucleic acid molecules or polypeptides mean that the nucleic acid molecule or the polypeptide is at least substantially free from at least one other component with which they are naturally associated in nature and as found in nature.

The term “effective amount” or “therapeutically effective amount” refers to the amount of an agent that is sufficient to effect beneficial or desired results. The therapeutically effective amount may vary depending upon one or more of: the subject and disease condition being treated, the weight and age of the subject, the severity of the disease condition, the manner of administration and the like, which can readily be determined by one of ordinary skill in the art. The term also applies to a dose that will provide an image for detection by any one of the imaging methods described herein. The specific dose may vary depending on one or more of: the particular agent chosen, the dosing regimen to be followed, whether it is administered in combination with other compounds, timing of administration, the tissue to be imaged, and the physical delivery system in which it is carried.

The practice of the present invention employs, unless otherwise indicated, conventional techniques of immunology, biochemistry, chemistry, molecular biology, microbiology, cell biology, genomics and recombinant DNA, which are within the skill of the art. See Sambrook, Fritsch and Maniatis, MOLECULAR CLONING: A LABORATORY MANUAL, 2nd edition (1989); CURRENT PROTOCOLS IN MOLECULAR BIOLOGY (F. M. Ausubel, et al. eds., (1987)); the series METHODS IN ENZYMOLOGY (Academic Press, Inc.): PCR 2: A PRACTICAL APPROACH (M. J. MacPherson, B. D. Hames and G. R. Taylor eds. (1995)), Harlow and Lane, eds. (1988) ANTIBODIES, A LABORATORY MANUAL, and ANIMAL CELL CULTURE (R. I. Freshney, ed. (1987)).

In some embodiments, a vector is capable of driving expression of one or more sequences in mammalian cells using a mammalian expression vector. Examples of mammalian expression vectors include pCDM8 (Seed, 1987. Nature 329: 840) and pMT2PC (Kaufman, et al., 1987. EMBO J. 6: 187-195). When used in mammalian cells, the expression vector's control functions are typically provided by one or more regulatory elements. For example, commonly used promoters are derived from polyoma, adenovirus 2, cytomegalovirus, simian virus 40, and others disclosed herein and known in the art. For other suitable expression systems for both prokaryotic and eukaryotic cells see, e.g., Chapters 16 and 17 of Sambrook, et al., MOLECULAR CLONING: A LABORATORY MANUAL. 2nd ed., Cold Spring Harbor Laboratory, Cold Spring Harbor Laboratory Press, Cold Spring Harbor, N.Y., 1989.

The term “vector” relates to a single or double stranded circular nucleic acid molecule that can be infected, transfected or transformed into cells and replicate independently or within the host cell genome. A circular double stranded nucleic acid molecule can be cut and thereby linearized upon treatment with restriction enzymes. An assortment of vectors, restriction enzymes, and the knowledge of the nucleotide sequences that are targeted by restriction enzymes are readily available to those skilled in the art, and include any replicon, such as a plasmid, cosmid, bacmid, phage or virus, to which another genetic sequence or element (either DNA or RNA) may be attached so as to bring about the replication of the attached sequence or element. A nucleic acid molecule of the invention can be inserted into a vector by cutting the vector with restriction enzymes and ligating the two pieces together.

In some embodiments, the recombinant mammalian expression vector is capable of directing expression of the nucleic acid (e.g., an antisense oligonucleotide) preferentially in a particular cell type (e.g., tissue-specific regulatory elements are used to express the nucleic acid). Tissue-specific regulatory elements are known in the art. Non-limiting examples of suitable tissue-specific promoters include the albumin promoter (liver-specific; Pinkert, et al., 1987. Genes Dev. 1: 268-277), lymphoid-specific promoters (Calame and Eaton, 1988. Adv. Immunol. 43: 235-275), in particular promoters of T cell receptors (Winoto and Baltimore, 1989. EMBO J. 8: 729-733) and immunoglobulins (Baneiji, et al., 1983. Cell 33: 729-740; Queen and Baltimore, 1983. Cell 33: 741-748), neuron-specific promoters (e.g., the neurofilament promoter; Byrne and Ruddle, 1989. Proc. Natl. Acad. Sci. USA 86: 5473-5477), pancreas-specific promoters (Edlund, et al., 1985. Science 230: 912-916), and mammary gland-specific promoters (e.g., milk whey promoter, U.S. Pat. No. 4,873,316 and European Application Publication No. 264,166). Developmentally-regulated promoters are also encompassed, e.g., the murine hox promoters (Kessel and Gruss, 1990. Science 249: 374-379) and the α-fetoprotein promoter (Campes and Tilghman, 1989. Genes Dev. 3: 537-546). In order to obtain high levels of expression, the TUBB4A encoding nucleic acid can be codon-optimized.

Methods of non-viral delivery of nucleic acids include lipofection, nucleofection, microinjection, biolistics, virosomes, liposomes, immunoliposomes, polycation or lipid:nucleic acid conjugates, naked DNA, artificial virions, and agent-enhanced uptake of DNA. Lipofection is described in e.g., U.S. Pat. Nos. 5,049,386, 4,946,787; and 4,897,355) and lipofection reagents are sold commercially (e.g., Transfectam™ and Lipofectin™). Cationic and neutral lipids that are suitable for efficient receptor-recognition lipofection of polynucleotides include those of Feigner, WO 91/17424; WO 91/16024. Delivery can be to cells (e.g. in vitro or ex vivo administration) or target tissues (e.g. in vivo administration).

The preparation of lipid:nucleic acid complexes, including targeted liposomes such as immunolipid complexes, is well known to one of skill in the art (see, e.g., Crystal, Science 270:404-410 (1995); Banskota et al., Cell 185:250-265 (2022), Blaese et al., Cancer Gene Ther. 2:291-297 (1995); Behr et al., Bioconjugate Chem. 5:382-389 (1994); Remy et al., Bioconjugate Chem. 5:647-654 (1994); Gao et al., Gene Therapy 2:710-722 (1995); Ahmad et al., Cancer Res. 52:4817-4820 (1992); U.S. Pat. Nos. 4,186,183, 4,217,344, 4,235,871, 4,261,975, 4,485,054, 4,501,728, 4,774,085, 4,837,028, and 4,946,787).

The use of RNA or DNA viral based systems for the delivery of nucleic acids take advantage of highly evolved processes for targeting a virus to specific cells in the body and trafficking the viral payload to the nucleus. Viral vectors can be administered directly to patients (in vivo) or they can be used to treat cells in vitro, and the modified cells may optionally be administered to patients (ex vivo). Conventional viral based systems could include retroviral, lentivirus, adenoviral, adeno-associated and herpes simplex virus vectors for gene transfer. Integration in the host genome is possible with the retrovirus, lentivirus, and adeno-associated virus gene transfer methods, often resulting in long term expression of the inserted transgene. Additionally, high transduction efficiencies have been observed in many different cell types and target tissues.

The tropism of a retrovirus can be altered by incorporating foreign envelope proteins, expanding the potential target population of target cells. Lentiviral vectors are retroviral vectors that are able to transduce or infect non-dividing cells and typically produce high viral titers. Selection of a retroviral gene transfer system would therefore depend on the target tissue. Retroviral vectors are comprised of cis-acting long terminal repeats with packaging capacity for up to 6-10 kb of foreign sequence. The minimum cis-acting LTRs are sufficient for replication and packaging of the vectors, which are then used to integrate the therapeutic gene into the target cell to provide permanent transgene expression. Widely used retroviral vectors include those based upon murine leukemia virus (MuLV), gibbon ape leukemia virus (GaLV), Simian Immuno deficiency virus (SIV), human immuno deficiency virus (HIV), and combinations thereof (see, e.g., Buchscher et al., J. Virol. 66:2731-2739 (1992); Johann et al., J. Virol. 66:1635-1640 (1992); Sommnerfelt et al., Virol. 176:58-59 (1990); Wilson et al., J. Virol. 63:2374-2378 (1989); Miller et al., J. Virol. 65:2220-2224 (1991); PCT/US94/05700).

In applications where transient expression is preferred, adenoviral based systems may be used. Adenoviral based vectors are capable of very high transduction efficiency in many cell types and do not require cell division. With such vectors, high titer and levels of expression have been obtained. This vector can be produced in large quantities in a relatively simple system.

Adeno-associated virus (“AAV”) vectors may also be used to transduce cells with target nucleic acids, e.g., in the in vitro production of nucleic acids and peptides, and for in vivo and ex vivo gene therapy procedures (see, e.g., West et al., Virology 160:38-47 (1987); U.S. Pat. No. 4,797,368; WO 93/24641; Kotin, Human Gene Therapy 5:793-801 (1994); Muzyczka, J. Clin. Invest. 94:1351 (1994). Construction of recombinant AAV vectors are described in a number of publications, including U.S. Pat. No. 5,173,414; Tratschin et al., Mol. Cell. Biol. 5:3251-3260 (1985); Tratschin, et al., Mol. Cell. Biol. 4:2072-2081 (1984); Hermonat & Muzyczka, PNAS 81:6466-6470 (1984); and Samulski et al., J. Virol. 63:03822-3828 (1989).

Packaging cells are typically used to form virus particles that are capable of infecting a host cell. Such cells include 293 cells, which package adenovirus, and ψ2 cells or PA317 cells, which package retrovirus. Viral vectors used in gene therapy are usually generated by producing a cell line that packages a nucleic acid vector into a viral particle. The vectors typically contain the minimal viral sequences required for packaging and subsequent integration into a host, other viral sequences being replaced by an expression cassette for the polynucleotide(s) to be expressed. The missing viral functions are typically supplied in trans by the packaging cell line. For example, AAV vectors used in gene therapy typically only possess ITR sequences from the AAV genome which are required for packaging and integration into the host genome. Viral DNA is packaged in a cell line, which contains a helper plasmid encoding the other AAV genes, namely rep and cap, but lacking ITR sequences. The cell line may also be infected with adenovirus as a helper. The helper virus promotes replication of the AAV vector and expression of AAV genes from the helper plasmid. The helper plasmid is not packaged in significant amounts due to a lack of ITR sequences. Contamination with adenovirus can be reduced by, e.g., heat treatment to which adenovirus is more sensitive than AAV.

In some embodiments, a host cell is transiently or non-transiently transfected with one or more vectors described herein. In some embodiments, a cell is transfected as it naturally occurs in a subject. In some embodiments, a cell that is transfected is taken from a subject. In some embodiments, the cell is derived from cells taken from a subject, such as a cell line.

In one aspect, the invention provides for methods of modifying a target polynucleotide in a eukaryotic cell, which may be in vivo, ex vivo or in vitro. In some embodiments, the method comprises sampling a cell or population of cells from a human or non-human animal, and modifying the cell or cells. Culturing may occur at any stage ex vivo. The cell or cells may be re-introduced into the human or non-human animal.

In one aspect, the invention provides for methods of modifying a target polynucleotide in a eukaryotic cell. In some embodiments, the method comprises allowing a CRISPR complex to bind to the target polynucleotide to effect cleavage of said target polynucleotide thereby modifying the target polynucleotide, wherein the CRISPR complex comprises a CRISPR enzyme complexed with a guide sequence hybridized to a target sequence within said target polynucleotide, wherein said guide sequence is linked to a tracr mate sequence which in turn hybridizes to a tracr sequence.

In one aspect, the invention provides kits containing any one or more of the elements disclosed in the above methods and compositions. In some embodiments, the kit comprises a vector system or components for an alternative delivery system such as those described above. Elements may be provided individually or in combinations, and may be provided in any suitable container, such as a vial, a bottle, or a tube. In some embodiments, the kit includes instructions in one or more languages, for example in more than one language.

In some embodiments, a kit comprises one or more reagents for use in a process utilizing one or more of the elements described herein. Reagents may be provided in any suitable container. For example, a kit may provide one or more reaction or storage buffers. Reagents may be provided in a form that is usable in a particular assay, or in a form that requires addition of one or more other components before use (e.g. in concentrate or lyophilized form). A buffer can be any buffer, including but not limited to a sodium carbonate buffer, a sodium bicarbonate buffer, a borate buffer, a Tris buffer, a MOPS buffer, a HEPES buffer, and combinations thereof. In some embodiments, the buffer is alkaline. In some embodiments, the buffer has a pH from about 7 to about 10. In some embodiments, the kit comprises one or more oligonucleotides corresponding to a guide sequence for insertion into a vector so as to operably link the guide sequence and a regulatory element. In some embodiments, the kit comprises a homologous recombination template polynucleotide.

Down-modulating or inhibitory nucleic acids include, without limitation, antisense molecules, aptamers, ribozymes, triplex forming molecules, RNA interference (RNAi), CRISPR (Clustered Regularly Interspaced Short Palindromic Repeats) RNA (crRNA), and external guide sequences. These nucleic acid molecules can act as affectors, inhibitors, modulators, and stimulators of a specific activity possessed by a target molecule, or the functional nucleic acid molecules can possess a de novo activity independent of any other molecules. In certain embodiments, inhibitory nucleic acids are employed.

Antisense molecules are designed to interact with a target nucleic acid molecule through either canonical or non-canonical base pairing. The interaction of the antisense molecule and the target molecule is designed to promote the destruction of the target molecule through, for example, RNase H mediated RNA-DNA hybrid degradation. Alternatively, the antisense molecule is designed to interrupt a processing function that normally would take place on the target molecule, such as transcription or replication. Antisense molecules can be designed based on the sequence of the target molecule. Numerous methods for optimization of antisense efficiency by finding the most accessible regions of the target molecule exist. Exemplary methods would be in vitro selection experiments and DNA modification studies using DMS and DEPC. It is preferred that antisense molecules bind the target molecule with a dissociation constant (K_{d}) less than or equal to 10⁻⁶, 10⁻⁸, 10⁻¹⁰, or 10⁻¹². A representative sample of methods and techniques which aid in the design and use of antisense molecules can be found in U.S. Pat. Nos. 5,135,917, 5,294,533, 5,627,158, 5,641,754, 5,691,317, 5,780,607, 5,786,138, 5,849,903, 5,856,103, 5,919,772, 5,955,590, 5,990,088, 5,994,320, 5,998,602, 6,005,095, 6,007,995, 6,013,522, 6,017,898, 6,018,042, 6,025,198, 6,033,910, 6,040,296, 6,046,004, 6,046,319, and 6,057,437.

Triplex forming functional nucleic acid molecules are molecules that can interact with either double-stranded or single-stranded nucleic acid. When triplex molecules interact with a target region, a structure called a triplex is formed, in which there are three strands of DNA forming a complex dependent on both Watson-Crick and Hoogsteen base-pairing. Triplex molecules are preferred because they can bind target regions with high affinity and specificity. It is preferred that the triplex forming molecules bind the target molecule with a K_{d} less than 10⁻⁶, 10⁻⁸, 10⁻¹⁰, or 10⁻¹². Representative examples of how to make and use triplex forming molecules to bind a variety of different target molecules can be found in U.S. Pat. Nos. 5,176,996, 5,645,985, 5,650,316, 5,683,874, 5,693,773, 5,834,185, 5,869,246, 5,874,566, and 5,962,426. Gene expression can also be effectively silenced in a highly specific manner through RNA interference (RNAi). This silencing was originally observed with the addition of double stranded RNA (dsRNA) (Fire, A., et al., Nature, 391:806-11 (1998); Napoli, C., et al., Plant Cell, 2:279-89 (1990); Hannon, G. J., Nature, 418:244-51 (2002)). Once dsRNA enters a cell, it is cleaved by an RNase III-like enzyme, Dicer, into double stranded small interfering RNAs (siRNA) 21-23 nucleotides in length that contain 2 nucleotide overhangs on the 3′ ends (Elbashir, S. M., et al., Genes Dev., 15:188-200 (2001); Bernstein, E., et al., Nature, 409:363-6 (2001); Hammond, S. M., et al., Nature, 404:293-6 (2000)). In an ATP-dependent step, the siRNAs become integrated into a multi-subunit protein complex, commonly known as the RNAi induced silencing complex (RISC), which guides the siRNAs to the target RNA sequence (Nykanen, A., et al., Cell, 107:309-21 (2001)). At some point the siRNA duplex unwinds, and it appears that the antisense strand remains bound to RISC and directs degradation of the complementary mRNA sequence by a combination of endo and exonucleases (Martinez, J., et al., Cell, 110:563-74 (2002)). However, the effect of RNAi or siRNA or their use is not limited to any type of mechanism.

Small Interfering RNA (siRNA) is a double-stranded RNA that can induce sequence-specific post-transcriptional gene silencing, thereby decreasing or even inhibiting gene expression. In one example, an siRNA triggers the specific degradation of homologous RNA molecules, such as mRNAs, within the region of sequence identity between both the siRNA and the target RNA. For example, WO 02/44321 discloses siRNAs capable of sequence-specific degradation of target mRNAs when base-paired with 3′ overhanging ends, herein incorporated by reference for the method of making these siRNAs. Sequence specific gene silencing can be achieved in mammalian cells using synthetic, short double-stranded RNAs that mimic the siRNAs produced by the enzyme dicer (Elbashir, S. M., et al., Nature, 411:494 498(2001); Ui-Tei, K., et al., FEBS Lett, 479:79-82 (2000)). siRNA can be chemically or in vitro-synthesized or can be the result of short double-stranded hairpin-like RNAs (shRNAs) that are processed into siRNAs inside the cell. Synthetic siRNAs are generally designed using algorithms and a conventional DNA/RNA synthesizer. Suppliers include Ambion (Austin, Tex.), ChemGenes (Ashland, Mass.), Dharmacon (Lafayette, Colo.), Glen Research (Sterling, Va.), MWB Biotech (Esbersberg, Germany), Proligo (Boulder, Colo.), and Qiagen (Vento, The Netherlands). siRNA can also be synthesized in vitro using kits such as Ambion's SILENCER® siRNA

As used herein, the term “overexpressing” when referring to the production of a protein in a host cell means that the protein is produced in greater amounts than it is produced in its naturally occurring environment.

The term “genetic alteration” as used herein refers to a change from the wild-type or reference sequence of one or more nucleic acid molecules. Genetic alterations include without limitation, base pair substitutions, additions and deletions of at least one nucleotide from a nucleic acid molecule of known sequence.

The term “solid matrix” as used herein refers to any format, such as beads, microparticles, a microarray, the surface of a microtitration well or a test tube, a dipstick or a filter. The material of the matrix may be polystyrene, cellulose, latex, nitrocellulose, nylon, polyacrylamide, dextran or agarose.

“Target nucleic acid” as used herein refers to a previously defined region of a nucleic acid present in a complex nucleic acid mixture wherein the defined wild-type region contains at least one known nucleotide variation associated with leukodystrophy. The nucleic acid molecule may be isolated from a natural source by cDNA cloning or subtractive hybridization or synthesized manually. The nucleic acid molecule may be synthesized manually by the triester synthetic method or by using an automated DNA synthesizer.

The term “complementary” describes two nucleotides that can form multiple favorable interactions with one another. For example, adenine is complementary to thymine as they can form two hydrogen bonds. Similarly, guanine and cytosine are complementary since they can form three hydrogen bonds. Thus, if a nucleic acid sequence contains the following sequence of bases, thymine, adenine, guanine and cytosine, a “complement” of this nucleic acid molecule would be a molecule containing adenine in the place of thymine, thymine in the place of adenine, cytosine in the place of guanine, and guanine in the place of cytosine. Because the complement can contain a nucleic acid sequence that forms optimal interactions with the parent nucleic acid molecule, such a complement can bind with high affinity to its parent molecule.

The term “promoter element” describes a nucleotide sequence that is incorporated into a vector that, once inside an appropriate cell, can facilitate transcription factor and/or polymerase binding and subsequent transcription of portions of the vector DNA into mRNA. In one embodiment, the promoter element of the present invention precedes the 5′ end of the Leukodystrophy specific marker nucleic acid molecule such that the latter is transcribed into mRNA. Host cell machinery then translates mRNA into a polypeptide.

Those skilled in the art will recognize that a nucleic acid vector can contain nucleic acid elements other than the promoter element and the TUBB4A down modulating nucleic acid molecule. These other nucleic acid elements include, but are not limited to, origins of replication, ribosomal binding sites, nucleic acid sequences encoding drug resistance enzymes or amino acid metabolic enzymes, and nucleic acid sequences encoding secretion signals, localization signals, or signals useful for polypeptide purification.

A “replicon” is any genetic element, for example, a plasmid, cosmid, bacmid, plastid, phage or virus, that is capable of replication largely under its own control. A replicon may be either RNA or DNA and may be single or double stranded.

An “expression operon” refers to a nucleic acid segment that may possess transcriptional and translational control sequences, such as promoters, enhancers, translational start signals (e.g., ATG or AUG codons), polyadenylation signals, terminators, and the like, and which facilitate the expression of a polypeptide coding sequence in a host cell or organism.

As used herein, the terms “reporter,” “reporter system”, “reporter gene,” or “reporter gene product” shall mean an operative genetic system in which a nucleic acid comprises a gene that encodes a product that when expressed produces a reporter signal that is a readily measurable, e.g., by biological assay, immunoassay, radio immunoassay, or by colorimetric, fluorogenic, chemiluminescent or other methods. The nucleic acid may be either RNA or DNA, linear or circular, single or double stranded, antisense or sense polarity, and is operatively linked to the necessary control elements for the expression of the reporter gene product. The required control elements will vary according to the nature of the reporter system and whether the reporter gene is in the form of DNA or RNA, but may include, but not be limited to, such elements as promoters, enhancers, translational control sequences, poly A addition signals, transcriptional termination signals and the like.

As mentioned above, the introduced nucleic acid may or may not be integrated (covalently linked) into nucleic acid of the recipient cell or organism. In bacterial, yeast, plant and mammalian cells, for example, the introduced nucleic acid may be maintained as an episomal element or independent replicon such as a plasmid. Alternatively, the introduced nucleic acid may become integrated into the nucleic acid of the recipient cell or organism and be stably maintained in that cell or organism and further passed on or inherited to progeny cells or organisms of the recipient cell or organism. Finally, the introduced nucleic acid may exist in the recipient cell or host organism only transiently.

The term “operably linked” means that the regulatory sequences necessary for expression of the coding sequence are placed in the DNA molecule in the appropriate positions relative to the coding sequence so as to effect expression of the coding sequence. This same definition is sometimes applied to the arrangement of transcription units and other transcription control elements (e.g. enhancers) in an expression vector.

“Gymnosis” as described herein entails delivery of single-stranded antisense oligodeoxynucleotides to cells in the absence of any carriers (example: transfection) or conjugation, that produces sequence-specific gene silencing.

The phrase “modified backbone linkage”, includes but is not limited to phosphorothioate linkages, methylphosphonate linkages, ethylphosphonate linkages, boranophosphate linkages, sulfonamide, carbonylamide, phosphorodiamidate, phosphorodiamidate linkages comprising a positively charged side group, phosphorodithioates, aminoethylglycine, phosphotriesters, aminoalkylphosphotriesters; 3′-alkylene phosphonates; 5′-alkylene phosphonates, chiral phosphonates, phosphinates, 3′-amino phosphoramidate, aminoalkylphosphoramidates, thionophosphoramidates; thionoalkyl-phosphonates, thionoalkylphosphotriesters, selenophosphates, 2-5′ linked boranophosphonate analogs, linkages having inverted polarity, abasic linkages, short chain alkyl linkages, cycloalkyl internucleoside linkages, mixed heteroatom and alkyl or cycloalkyl internucleoside linkages, short chain heteroatomic or heterocyclic internucleoside linkages with siloxane backbones, sulfide, sulfoxide, sulfone, formacetyl linkages, thioformacetyl linkages, methylene formacetyl linkages, thioformacetyl linkages, riboacetyl linkages, alkene linkages, sulfamate backbones, methyleneimino linkages, methylenehydrazino linkages, sulfonate linkages, and amide linkages.

The phrase “modified sugar” includes, without limitation, 2′ fluoro, 2′ fluoro substituted ribose, 2′-fluoro-D-arabinonucleic acid (FANA), 2′-0-methoxyethyl ribose, 2′-O-methoxyethyl deoxyribose, 2′-O-methyl substituted ribose, a morpholino, a piperazine, and a locked nucleic acid (LNA).

A “specific binding pair” comprises a specific binding member (sbm) and a binding partner (bp) which have a particular specificity for each other and which in normal conditions bind to each other in preference to other molecules. Examples of specific binding pairs are antigens and antibodies, ligands and receptors and complementary nucleotide sequences. The skilled person is aware of many other examples. Further, the term “specific binding pair” is also applicable where either or both of the specific binding member and the binding partner comprise a part of a large molecule. In embodiments in which the specific binding pair comprises nucleic acid sequences, they will be of a length to hybridize to each other under conditions of the assay, preferably greater than 10 nucleotides long, more preferably greater than 15 or 20 nucleotides long.

“Sample” or “patient sample” or “biological sample” generally refers to a sample which may be tested for a particular molecule, preferably an leukodystrophy specific marker molecule, such as a marker shown in the tables provided below. Samples may include but are not limited to cells, body fluids, including blood, serum, plasma, urine, saliva, cerebral spinal fluid, tears, pleural fluid and the like.

### Kits and Articles of Manufacture

Any of the aforementioned products can be incorporated into a kit which may contain a TUBB4A directed down modulating nucleic acids in pharmaceutically acceptable carrier. The nucleic acid may or may not be disposed in a vector which is capable of transducing mammalian cells. In other aspects, the kit contains a vector which expresses human wild type TUBB4A and/or mutant TUBB4A encoding nucleic acids for overexpressing the same in target cells of interest. The kit may optionally include nanoparticle or liposome formulations which facilitate delivery of the nucleic acids into cells. The kit may also contain instructions for use, a container, a vessel for administration, an assay substrate, or any combination thereof.

### Methods for the Development and Screening of Therapeutic Agents

Since genetic alterations in TUBB4A identified herein have been associated with the etiology of H-ABC, methods for identifying agents that modulate the activity of the mutated genes and their encoded products should result in the generation of efficacious therapeutic agents for the treatment of leukodystrophy, particularly H-ABC.

Molecular modeling should facilitate the identification of specific organic molecules with capacity to bind to the active site of altered TUBB4A proteins based on conformation or key amino acid residues required for function. A combinatorial chemistry approach will be used to identify molecules with greatest activity and then iterations of these molecules will be developed for further cycles of screening.

The polypeptides or fragments employed in drug screening assays may either be free in solution, affixed to a solid support or within a cell. One method of drug screening utilizes eukaryotic or prokaryotic host cells which are stably transformed with recombinant polynucleotides expressing the polypeptide or fragment, preferably in competitive binding assays. Such cells, either in viable or fixed form, can be used for standard binding assays. One may determine, for example, formation of complexes between the polypeptide or fragment and the agent being tested, or examine the degree to which the formation of a complex between the polypeptide or fragment and a known substrate is interfered with by the agent being tested.

Another technique for drug screening provides high throughput screening for compounds having suitable binding affinity for the encoded polypeptides and is described in detail in Geysen, PCT published application WO 84/03564, published on Sep. 13, 1984. Briefly stated, large numbers of different, small peptide test compounds, such as those described above, are synthesized on a solid substrate, such as plastic pins or some other surface. The peptide test compounds are reacted with the target polypeptide and washed. Bound polypeptide is then detected by methods well known in the art.

A further technique for drug screening involves the use of host eukaryotic cell lines or cells (such as described above) which have a nonfunctional or altered TUBB4A associated gene. These host cell lines or cells are defective at the polypeptide level. The host cell lines or cells are grown in the presence of drug compound. The rate of cellular metabolism of the host cells is measured to determine if the compound is capable of regulating the cellular metabolism in the defective cells. Methods for introducing DNA molecules are also well known to those of ordinary skill in the art as discussed above.

Host cells expressing the H-ABC associated nucleic acids of the present invention or functional fragments thereof provide a system in which to screen potential compounds or agents for the ability to modulate the development of leukodystrophy. Thus, in one embodiment, the nucleic acid molecules of the invention may be used to create recombinant cell lines for use in assays to identify agents which modulate aspects of cellular metabolism associated with neuronal signaling and neuronal cell communication and structure. Also provided herein are methods to screen for compounds capable of modulating the function of proteins encoded by TUBB4A containing nucleic acids.

Another approach entails the use of phage display libraries engineered to express fragment of the polypeptides encoded by the altered TUBB4A nucleic acids on the phage surface. Such libraries are then contacted with a combinatorial chemical library under conditions wherein binding affinity between the expressed peptide and the components of the chemical library may be detected. U.S. Pat. Nos. 6,057,098 and 5,965,456 provide methods and apparatus for performing such assays. Such compound libraries are commercially available from a number of companies including but not limited to Maybridge Chemical Co., (Trevillet, Cornwall, UK), Comgenex (Princeton, N.J.), Microsour (New Milford, Conn.) Aldrich (Milwaukee, Wis.) Akos Consulting and Solutions GmbH (Basel, Switzerland), Ambinter (Paris, France), Asinex (Moscow, Russia) Aurora (Graz, Austria), BioFocus DPI (Switzerland), Bionet (Camelford, UK), Chembridge (San Diego, Calif.), Chem Div (San Diego, Calif.). The skilled person is aware of other sources and can readily purchase the same. Once therapeutically efficacious compounds are identified in the screening assays described herein, they can be formulated into pharmaceutical compositions and utilized for the treatment of H-ABC.

The goal of rational drug design is to produce structural analogs of biologically active polypeptides of interest or of small molecules with which they interact (e.g., agonists, antagonists, inhibitors) in order to fashion drugs which are, for example, more active or stable forms of the polypeptide, or which, e.g., enhance or interfere with the function of a polypeptide in vivo. See, e.g., Hodgson, (1991) Bio/Technology 9:19-21. In one approach, discussed above, the three-dimensional structure of a protein of interest or, for example, of the protein-substrate complex, is solved by x-ray crystallography, by nuclear magnetic resonance, by computer modeling or most typically, by a combination of approaches. Less often, useful information regarding the structure of a polypeptide may be gained by modeling based on the structure of homologous proteins. An example of rational drug design is the development of HIV protease inhibitors (Erickson et al., (1990) Science 249:527-533). In addition, peptides may be analyzed by an alanine scan (Wells, (1991) Meth. Enzym. 202:390-411). In this technique, an amino acid residue is replaced by Ala, and its effect on the peptide's activity is determined. Each of the amino acid residues of the peptide is analyzed in this manner to determine the important regions of the peptide.

It is also possible to isolate a target-specific antibody, selected by a functional assay, and then to solve its crystal structure. In principle, this approach yields a pharmacore upon which subsequent drug design can be based.

One can bypass protein crystallography altogether by generating anti-idiotypic antibodies (anti-ids) to a functional, pharmacologically active antibody. As a mirror image of a mirror image, the binding site of the anti-ids would be expected to be an analog of the original molecule. The anti-id could then be used to identify and isolate peptides from banks of chemically or biologically produced banks of peptides. Selected peptides would then act as the pharmacore.

In another embodiment, the availability of altered TUBB4A nucleic acids enables the production of strains of laboratory mice carrying the leukodystrophy-associated TUBB4A nucleic acids of the invention as described herein below. Transgenic mice expressing the leukodystrophy-associated nucleic acids of the invention provide a model system in which to examine the role of the mutated TUBB4A protein in the development and progression towards leukodystrophy. Methods of introducing transgenes in laboratory mice are known to those of skill in the art and are described hereinbelow. Three common methods include: 1. integration of retroviral vectors encoding the foreign gene of interest into an early embryo; 2. injection of DNA into the pronucleus of a newly fertilized egg; and 3. the incorporation of genetically manipulated embryonic stem cells into an early embryo. Production of the transgenic mice described above will facilitate the molecular elucidation of the role that a target protein plays in various cellular metabolic and neuronal processes. Such mice provide an in vivo screening tool to study putative therapeutic drugs in a whole animal model and are encompassed by the present invention.

The term “animal” is used herein to include all vertebrate animals, except humans. It also includes an individual animal in all stages of development, including embryonic and fetal stages. A “transgenic animal” is any animal containing one or more cells bearing genetic information altered or received, directly or indirectly, by deliberate genetic manipulation at the subcellular level, such as by targeted recombination or microinjection or infection with recombinant virus. The term “transgenic animal” is not meant to encompass classical cross-breeding or in vitro fertilization, but rather is meant to encompass animals in which one or more cells are altered by or receive a recombinant DNA molecule. This molecule may be specifically targeted to a defined genetic locus, be randomly integrated within a chromosome, or it may be extrachromosomally replicating DNA. The term “germ cell line transgenic animal” refers to a transgenic animal in which the genetic alteration or genetic information was introduced into a germ line cell, thereby conferring the ability to transfer the genetic information to offspring. If such offspring, in fact, possess some or all of that alteration or genetic information, then they, too, are transgenic animals.

The DNA used for altering a target gene may be obtained by a wide variety of techniques that include, but are not limited to, isolation from genomic sources, preparation of cDNAs from isolated mRNA templates, direct synthesis, or a combination thereof.

A preferred type of target cell for transgene introduction is the embryonal stem cell (ES). ES cells may be obtained from pre-implantation embryos cultured in vitro (Evans et al., (1981) Nature 292:154-156; Bradley et al., (1984) Nature 309:255-258; Gossler et al., (1986) Proc. Natl. Acad. Sci. 83:9065-9069). Transgenes can be efficiently introduced into the ES cells by standard techniques such as DNA transfection or by retrovirus-mediated transduction. The resultant transformed ES cells can thereafter be combined with blastocysts from a non-human animal. The introduced ES cells thereafter colonize the embryo and contribute to the germ line of the resulting chimeric animal.

Techniques are available to inactivate or alter any genetic region to a mutation desired by As used herein, a knock-in animal is one in which the endogenous murine gene, for example, has been replaced with human leukodystrophy-associated TUBB4A gene of the invention. Such knock-in animals provide an ideal model system for studying the development of leukodystrophy. Knock-out animals can also be created.

As used herein, the expression of a leukodystrophy associated nucleic acid, fragment thereof, can be targeted in a “tissue specific manner” or “cell type specific manner” using a vector in which nucleic acid sequences encoding all or a portion of leukodystrophy-associated nucleic acids are operably linked to regulatory sequences (e.g., promoters and/or enhancers) that direct expression of the encoded protein in a particular tissue or cell type. Such regulatory elements may be used to advantage for both in vitro and in vivo applications. Promoters for directing tissue specific proteins are well known in the art and described herein.

Methods of use for the transgenic mice of the invention are also provided herein. Transgenic mice into which a nucleic acid containing the leukodystrophy-associated TUBB4A or its encoded protein have been introduced are useful, for example, to develop screening methods to screen therapeutic agents to identify those capable of modulating the development of leukodystrophy.

### Pharmaceutical Compositions

Pharmaceutical compositions containing a therapeutic, prophylactic, or diagnostic agent derivative, such as functional nucleic acid derivative, may be administered parenterally to subjects in need of such a treatment. Parenteral administration can be performed by subcutaneous, intramuscular or intravenous injection by means of a syringe, optionally a pen-like syringe. Alternatively, parenteral administration can be performed by means of an infusion pump. Further options are to administer the therapeutic, prophylactic, or diagnostic agent nasally or pulmonally, preferably in compositions, powders or liquids, specifically designed for the purpose.

Injectable compositions of the therapeutic, prophylactic, or diagnostic agent derivatives, including intracranial injection, can be prepared using the conventional techniques of the pharmaceutical industry which involve dissolving and mixing the ingredients as appropriate to give the desired end product. Thus, according to one procedure, a therapeutic, prophylactic, or diagnostic agent derivative can be dissolved in an amount of water which is somewhat less than the final volume of the composition to be prepared. An isotonic agent, a preservative and a buffer can be added as required and the pH value of the solution is adjusted—if necessary—using an acid, e.g., hydrochloric acid, or a base, e.g., aqueous sodium hydroxide, as needed. Finally, the volume of the solution can be adjusted with water to give the desired concentration of the ingredients.

In some embodiments, the buffer can be selected from the group consisting of sodium acetate, sodium carbonate, citrate, glycylglycine, histidine, glycine, lysine, arginine, sodium dihydrogen phosphate, disodium hydrogen phosphate, sodium phosphate, and tris(hydroxymethyl)-aminomethan, bicine, tricine, malic acid, succinate, maleic acid, fumaric acid, tartaric acid, aspartic acid or mixtures thereof. Each one of these specific buffers and their combinations constitutes an alternative embodiment.

The pharmaceutical formulations, which may conveniently be presented in unit dosage form, may be prepared according to conventional techniques well known in the pharmaceutical industry. Such techniques include the step of bringing into association the active ingredients with the pharmaceutical carrier(s) or excipient(s). In general, the formulations are prepared by uniformly and intimately bringing into association the active ingredients with liquid carriers, finely divided solid carriers, or both, and then, if necessary, shaping the product (e.g., into a specific particle size for delivery). In a preferred embodiment of the invention and/or embodiments thereof, the pharmaceutical formulations are prepared for intramuscular administration in an appropriate solvent, e.g., water or normal saline, possibly in a sterile formulation, with carriers or other agents.

A “pharmaceutical carrier” or “excipient” can be a pharmaceutically acceptable solvent, suspending agent or any other pharmacologically inert vehicle for delivering one or more nucleic acids to an animal and are known in the art. The excipient may be liquid or solid and is selected, with the planned manner of administration in mind, so as to provide for the desired bulk, consistency, etc., when combined with a nucleic acid and the other components of a given pharmaceutical composition.

Compositions provided herein may contain two or more antisense compounds. In another related embodiment, compositions may contain one or more antisense compounds, particularly oligonucleotides, targeted to a first nucleic acid and one or more additional antisense compounds targeted to a second nucleic acid target. Alternatively, compositions provided herein can contain two or more antisense compounds targeted to different regions of the same nucleic acid target. Two or more combined compounds may be used together or sequentially. Compositions can also be combined with other non-antisense compound therapeutic agents.

The antisense oligomeric compound described herein may be in admixture with excipients suitable for the manufacture of aqueous suspensions. Such excipients are suspending agents, for example sodium carboxymethylcellulose, methylcellulose, hydropropyl-methylcellulose, sodium alginate, polyvinylpyrrolidone, gum tragacanth and gum acacia; dispersing or wetting agents can be a naturally-occurring phosphatide, for example, lecithin, or condensation products of an alkylene oxide with fatty acids, for example polyoxyethylene stearate, or condensation products of ethylene oxide with long chain aliphatic alcohols, for example heptadecaethyleneoxycetanol, or condensation products of ethylene oxide with partial esters derived from fatty acids and a hexitol such as polyoxyethylene sorbitol monooleate, or condensation products of ethylene oxide with partial esters derived from fatty acids and hexitol anhydrides, for example polyethylene sorbitan monooleate. Aqueous suspensions may also contain one or more preservatives, for example ethyl, or n-propyl p-hydroxybenzoate.

Dispersible powders and granules suitable for preparation of an aqueous suspension by the addition of water provide the active ingredient in admixture with a dispersing or wetting agent, suspending agent and one or more preservatives. antisense oligomeric compound compositions may be in the form of a sterile injectable aqueous or oleaginous suspension. Suspensions may be formulated according to the known art using those suitable dispersing or wetting agents and suspending agents that have been mentioned above. The sterile injectable preparation can also be a sterile injectable solution or suspension in a non-toxic parentally acceptable diluent or solvent, for example as a solution in 1,3-butanediol. Among the acceptable vehicles and solvents that can be employed are water, Ringer's solution and isotonic sodium chloride solution. In addition, sterile, fixed oils are conventionally employed as a solvent or suspending medium. For this purpose, any bland fixed oil can be employed including synthetic mono or diglycerides. In addition, fatty acids such as oleic acid find use in the preparation of injectables.

The present disclosure also includes antisense oligomeric compound compositions prepared for storage or administration that include a pharmaceutically effective amount of the desired compounds in a pharmaceutically acceptable carrier or diluent. Acceptable carriers or diluents for therapeutic use are well known in the pharmaceutical art, and are described, for example, in Remington's Pharmaceutical Sciences (Mack Publishing Co., A. R. Gennaro edit., 1985). For example, preservatives and stabilizers can be provided. These include sodium benzoate, sorbic acid and esters of p-hydroxybenzoic acid. In addition, antioxidants and suspending agents can be used.

Pharmaceutical compositions of this disclosure can also be in the form of oil-in-water emulsions. The oily phase can be a vegetable oil or a mineral oil or mixtures of these. Suitable emulsifying agents can be naturally-occurring gums, for example gum acacia or gum tragacanth, naturally-occurring phosphatides, for example soy bean, lecithin, and esters or partial esters derived from fatty acids and hexitol, anhydrides, for example sorbitan monooleate, and condensation products of the said partial esters with ethylene oxide, for example polyoxy ethylene sorbitan monooleate.

The antisense oligomeric compound of this disclosure may be administered to a patient by any standard means, with or without stabilizers, buffers, or the like, to form a composition suitable for treatment. When it is desired to use a liposome delivery mechanism, standard protocols for formation of liposomes can be followed. Thus the antisense oligomeric compound of the present disclosure may be administered in any form, for example intramuscular or by local, systemic, or intrathecal injection.

This disclosure also features the use of antisense oligomeric compound compositions comprising surface-modified liposomes containing poly(ethylene glycol) lipids (PEG-modif[iota]ed, or long-circulating liposomes or stealth liposomes). These formulations offer a method for increasing the accumulation of antisense oligomeric compound in target tissues. This class of drug carriers resists opsonization and elimination by the mononuclear phagocytic system (MPS or RES), thereby enabling longer blood circulation times and enhanced tissue exposure for the encapsulated antisense oligomeric compound (Lasic et al, Chem. Rev. 95:2601-2627 (1995) and Ishiwata et al, Chem. Pharm. Bull. 43:1005-1011 (1995). Long-circulating liposomes enhance the pharmacokinetics and pharmacodynamics of antisense oligomeric compound, particularly compared to conventional cationic liposomes which are known to accumulate in tissues of the MPS (Liu et al, J. Biol. Chem. 42:24864-24870 (1995); Choi et al, PCT Publication No. WO 96/10391; Ansell et al, PCT Publication No. WO 96/10390; Holland et al, PCT Publication No. WO 96/10392). Long-circulating liposomes are also likely to protect antisense oligomeric compound from nuclease degradation to a greater extent compared to cationic liposomes, based on their ability to avoid accumulation in metabolically aggressive MPS tissues such as the liver and spleen.

Following administration of the antisense oligomeric compound compositions according to the formulations and methods of this disclosure, test subjects will exhibit about a 10% up to about a 99% reduction in one or more symptoms associated with the disease or disorder being treated, as compared to placebo-treated or other suitable control subjects. Several methods for the delivery of antisense oligonucleotides for the treatment of neurodegenerative disorders are described in Evers et al. (2015) Advanced Drug Delivery Reviews 87:90-103, incorporated herein by reference.

The following materials and methods are provided to facilitate the practice of the present invention.

### Generation of Mouse Model

Heterozygous TUBB4A^{D249N} mice were generated using clustered regularly interspaced short palindromic repeats (CRISPR)—Cas-9 technology by inserting the p.Asp249Asn (c.745G>A) mutation in exon 4 of the TUBB4A gene. The mouse TUBB4A gene is located on Chromosome 17 comprising of 4 exons. Cas9 mRNA, gRNA and oligonucleotides (with targeting sequence, flanked by 120 bp homologous combined on both sides) were co-injected into zygotes. The resulting CRISPR knock-in mouse model has the heterozygous point mutation of c.745G>A in one allele of the TUBB4A gene (TUBB4A^{D249N}). These heterozygous mice were bred to produce homozygous TUBB4A^{D249N/D249N} mice in keeping with the homozygous mutation seen in taeip rat models (Li et al., 2003) in addition to heterozygous TUBB4A^{D249N} animals. Wild-type (WT), TUBB4A^{D249N} and TUBB4A^{D249N/D249N} mice were included in all analyses. The animals were genotyped at all experimental steps. Mice were maintained under a 12 h light:12 h dark cycle in a clean facility and given free access to food and water. The methods and study protocols conformed with the revised National Institutes of Health Office of Laboratory Animal Welfare Policy.

### Tissue Processing

Mice were deeply anesthetized based on weight with a mixture of 90-150 mg/kg of ketamine and 7.5-16 mg/kg of xylazine and transcardially perfused with 4% paraformaldehyde (PFA) in 1× phosphate buffer saline (PBS) (Thermo fisher Scientific, USA) after an initial flush with 1×PBS. Brains were collected and post-fixed with 4% PFA in 1×PBS overnight and then the tissues were dehydrated in 30% sucrose in 1×PBS. Brains were embedded in optimal cutting temperature compound (O.C.T. Compound, SAKURA, 4583, USA) and then sliced either as coronal or sagittal (50 m) sections on a cryostat microtome (CM 3050 S, Leica biosystems, USA).

### Antisense Oligonucleotide Synthesis and Screening

Mouse and human ASOs were designed and synthesized at Microsynth and Biospring companies. After receiving ASO in lyophilized powder form, ASOs were suspended in Tris-EDTA buffer (untreated vehicle) For both types of ASOs, in vitro screening was performed to identify the optimal ASO design. For screening of mouse ASOs, we used mouse HT-22 cells and for human ASO screening, we used human A5499 cells. One million cells of each cell line were electroporated with 1 μM, 5 μM and 10 μM of ASO concentrations at 150 V in 100 μL media with 100,000 cells/well on the NEPA21 electroporation system (NEPA GENE, USA). Following electroporation, cells were transferred to 96 well-plates and placed in an incubator. Forty-eight hours post-treatment, cells were washed with PBS and RNA extraction performed using PureLink™ RNA 96-well Kit (ThermoFisher Scientific, Cat #12173-011A) according to manufacturer's instructions. After treatment with DNAase (Invitrogen), 200 ng of RNA was used for cDNA with SuperScript™ IV First-Strand Synthesis System (ThermoFisher Scientific, Cat: 18091200). The mRNA expression levels of TUBB4A, and an endogenous housekeeping gene encoding Splicing factor, arginine/serine-rich 9 (sfrs9) as a reference, were quantified using real-time PCR analysis (Tagman chemistry) on an Applied Biosystems Quanta Flex 7 (ThermoFisher Scientific, USA). The results were analyzed using the ΔΔCT method.

### Mouse Intracerebroventricular (ICV) Injections

WT pups (P0-P1) were administered with the TUBB4A ASOs with different doses. ASOs were administered using a Hamilton 1700 gastight syringe (7653-01, Hamilton Company) by ICV injection to cryoanesthetized mice. The needle was placed between bregma and the eye, ⅖ the distance from bregma and inserted depth 2 mm. A total volume of 2 μL was administered to the left ventricle. Mice were allowed to recover on a heating pad and subsequently reintroduced to the dam.

Dose Response Determination Using RNA Extraction, cDNA Synthesis and qPCR

To determine the relative expression of TUBB4A in brain, after 10 days of ICV injection, mice were euthanized, and different brain areas were collected, snap frozen and stored at −80 C. RNA extraction was carried out using RNeasy 96 Universal Tissue kit (Qiagen, according to manufacturer's instructions. The mRNA expression levels of TUBB4A, and an endogenous housekeeping gene encoding hprt as a reference, were quantified using real-time PCR analysis (Tagman chemistry) on an Applied Biosystems Quanta Flex 7 (ThermoFisher Scientific, USA). The results were analyzed using the ΔΔCT method.

The following examples are provided to illustrate certain embodiments of the invention. They are not intended to limit the invention in any way.

### Example I

### Antisense Oligonucleotide Mediated Therapeutic Suppression of TUBB4A For the Treatment of H-ABC Leukodystrophy

Hypomyelination and Atrophy of Basal ganglia and Cerebellum (H-BC) is a rare leukodystrophy associated with mutations in tubular alpha 4A (TUBB4A). The p.Asp.249Asn (D249N) mutation is a recurring variant found in majority of H-ABC affected individuals. H-ABC typically begins in infancy, and is characterized by dystonia, ataxia, altered gait and progressive motor dysfunction. We recently characterized and published studies employing a CRISPR knock-in mouse model harboring this variant and recapitulating H-ABC disease features. Homozygous variants in TUBB4A^{D249N/D249N} exhibit progressive motor dysfunction, ataxia, decreed survival (˜P32-P37), severe myelination deficits and neuronal atrophy in striatum and cerebellum (Sase et al., 2020). When the variant is present only in the heterozygous state, TUBB4A^{D249N/+}, a reduced phenotype is observed characterized by a myelin defect without impact on motor abilities and survival. Thus, TUBB4A^{D249N/D249N} is a unique pre-clinical tool to test new therapeutic strategies for treatment of H-ABC disease. Our unpublished data shows that mice with germline deletion of both copies of TUBB4A (TUBB4A^{KO/KO}) develop normally, exhibit normal motor functions and show no myelination or neuronal deficits. When these TUBB4A^{KO/KO} are crossed with TUBB4A^{D249N/+} mice, the resulting TUBB4A^{D249N/KO} mice exhibit improved motor deficits, reduced myelination defects, decreased neuronal loss in the stratum and cerebellum, and increased survival (˜P110) relative to TUBB4A^{D249N/D249N} (n=8-9, p<0.0001). Together, these results suggest that H-ABC related disease correlates with overall expression of mutant TUBB4A an relative preservation of wild-type tubulin. Thus, we propose that one approach to treat H-ABC is to reduce overall Tubb4a expression. To evaluate the therapeutic potential of TUBB4A suppression, we designed new antisense oligonucleotides (ASO) targeted against human TUBB4A and mouse TUBB4A. We screened human TUBB4A ASOs in vitro in human cell line and found promising ASO sequences which reduces human TUBB4A. See Table 3 below.

TABLE 3
Human TUBB4A ASO sequences

| | | | | SEQ ID |
|---|---|---|---|---|
| | Label | Modified*AS*Seq | Length | NO: |
| 5240 | H1 | +U*+A*+G*G*T*C*T*C*A*T*C*C*G*+U*+A*+U* | 16 | 1 |
| 7652 | H2 | +U*+G*+C*A*C*G*C*T*C*A*G*C*A*+U*+C*+U* | 16 | 2 |
| 9913 | H3 | +U*+C*+A*G*A*A*G*C*C*T*C*G*+A*+G*+G* | 15 | 3 |
| 10479 | H4 | +G*+A*+G*+C*T*C*C*A*A*A*G*G*T*+A*+U*+U*+G* | 17 | 4 |
| 10491 | H5 | +G*+C*+G*+A*G*C*T*C*C*A*A*A*G*+G*+U*+A*+U* | 17 | 5 |
| 10502 | H6 | +A*+G*+C*G*A*G*C*T*C*C*A*A*A*+G*+G*+U* | 16 | 6 |
| 10532 | H7 | +G*+G*+U*A*A*A*G*C*G*A*G*C*T*+C*+C*+A* | 16 | 7 |
| 10563 | H8 | +G*+C*+C*+A*G*A*G*G*T*A*A*A*G*+C*+G*+A*+G* | 17 | 8 |
| 12277 | H9 | +G*+G*+U*T*A*A*A*G*G*T*G*A*+G*+G*+C* | 15 | 9 |
| 13004 | H10 | +G*+G*+A*C*T*T*G*C*A*G*G*T*G*+U*+A*+G* | 16 | 10 |
| 13231 | H11 | +G*+A*+A*C*T*G*C*A*G*C*T*C*+G*+G*+A* | 15 | 11 |
| 14024 | H12 | +A*+A*+G*+C*T*A*A*G*G*T*C*G*G*+C*+A*+G*+G* | 17 | 12 |
| 5230 | H13 | +U*+A*+G*+G*T*C*T*C*A*T*C*C*G*T*+A*+U*+U*+C* | 18 | 13 |
| 5241 | H14 | +G*+U*+A*+G*G*T*C*T*C*A*T*C*C*+G*+U*+A*+U* | 17 | 14 |
| 9925 | H15 | +G*+G*+U*C*A*G*A*A*G*C*C*T*+C*+G*+A* | 15 | 15 |
| 10486 | H16 | +G*+C*+G*+A*G*C*T*C*C*A*A*A*G*G*+U*+A*+U*+U* | 18 | 16 |
| 10492 | H17 | +A*+G*+C*+G*A*G*C*T*C*C*A*A*A*G*+G*+U*+A*+U* | 18 | 17 |
| 10497 | H18 | +A*+G*+C*+G*A*G*C*T*C*C*A*A*A*+G*+G*+U*+A* | 17 | 18 |
| 10504 | H19 | +A*+A*+A*+G*C*G*A*G*C*T*C*C*A*A*+A*+G*+G*+U* | 18 | 19 |
| 10509 | H20 | +A*+A*+A*+G*C*G*A*G*C*T*C*C*A*+A*+A*+G*+G* | 17 | 20 |
| 12999 | H21 | +G*+G*+A*+C*T*T*G*C*A*G*G*T*G*+U*+A*+G*+A* | 17 | 21 |
| 7652 | H2A | +U*+G+C+A | 16 | 22 |
| | | (5′Me)C*G*(5′Me)C*T*(5′Me)C*A*G*(5′Me)C*A+U+C+U | | |
| 7652 | H2B | +U*+G+C+A | 16 | 23 |
| | | (5′Me)C*G*(5′Me)C*T*(5′Me)C*A*G*(5′Me)C*A+U+C*+U | | |
| 13004 | H10A | +G*+G+A+CT*T*G*(5′Me)C*A*G*G*T*G+U+A+G | 16 | 24 |
| 13004 | H10B | +G*+G+A+CT*T*G*(5′Me)C*A*G*G*T*G+U+A*+G | 16 | 25 |
| 13231 | H11A | +G*+A+A(5′Me)C | 15 | 26 |
| | | T*G*(5′Me)C*A*G*(5′Me)C*T*(5′Me)C+G+G+A | | |
| 13231 | H11B | +G*+A+A (5′Me)C | 15 | 27 |
| | | T*G*(5′Me)C*A*G*(5′Me)C*T*(5′Me)C+G+G*+A | | |
| 9925 | H15A | +G*+G+U (5′Me)C | 15 | 28 |
| | | A*G*A*A*G*(5′Me)C*(5′Me)C*T+C+G+A | | |
| 9925 | H15B | +G*+G+U(5′Me)C | 15 | 29 |
| | | A*G*A*A*G*(5′Me)C*(5′Me)C*T+C+G*+A | | |

*phosphorothioate bonds
+sign-2′-O-(2-Methoxyethyl)-oligori-bonucleotides (2′-MOE)
5′Me-5′methyl

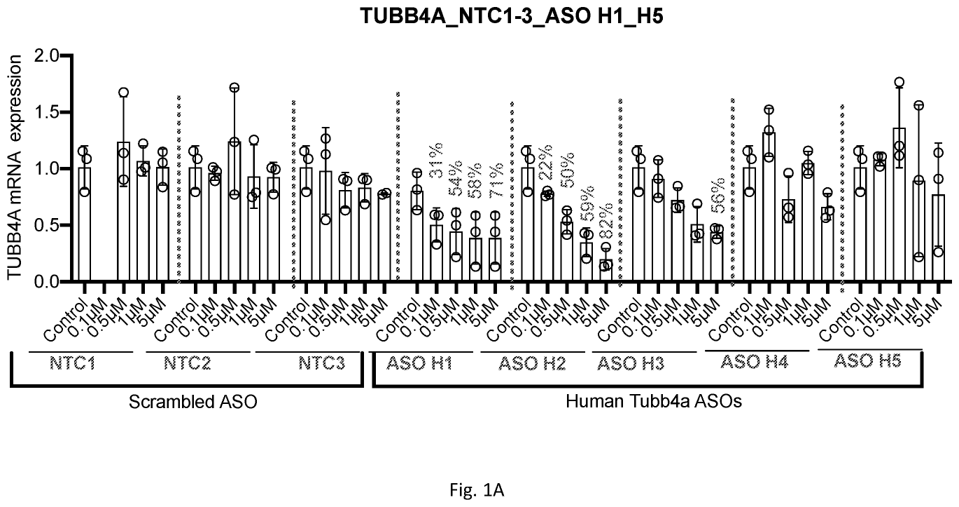

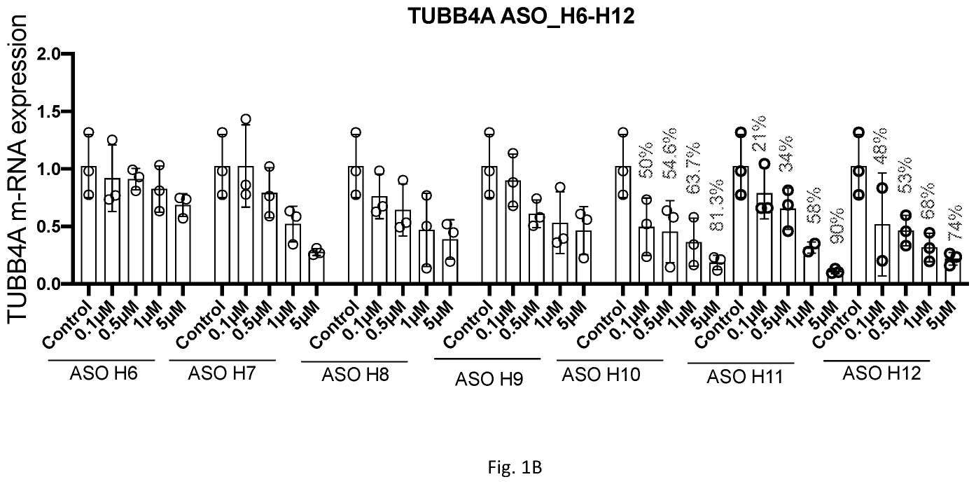

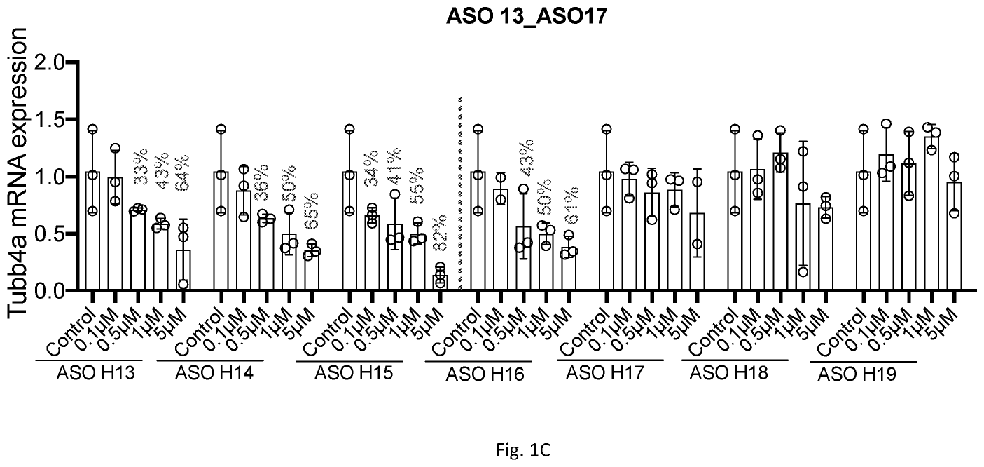

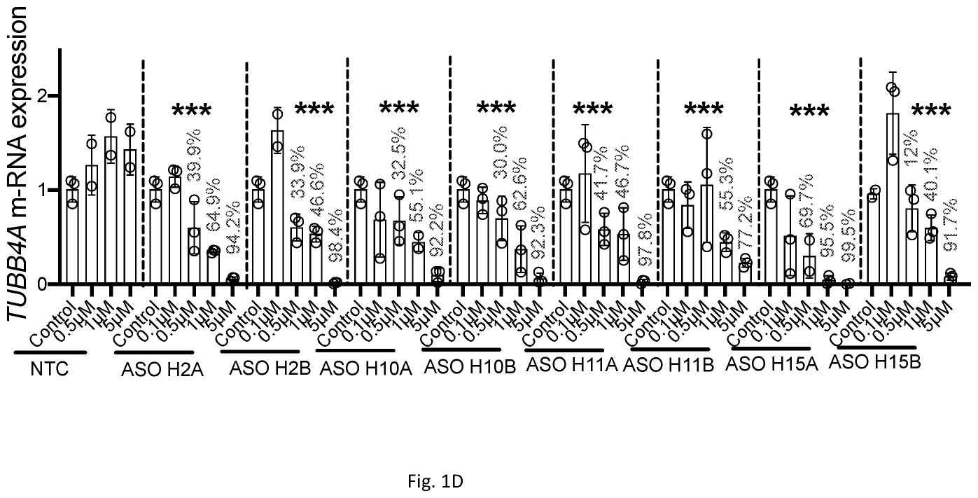

The library of ASOs was screened in A549N cells using electroporation with three replicates. After ASO electroporation at different concentrations (0.1 μM, 0.5 μM, 1 μM and M), RNA was extracted and subjected to qPCR to assess the TUBB4A expression. TUBB4A expression is normalized to GAPDH (n=3, ***p<0.0001). The results are shown in FIG. 1A-1D. A selection of these ASOs were further tested on induced pluripotent stem cell (iPSCs) generated from H-ABC patient cells below. These ASOs can be further tested using cells from patients with other TUBB4A mutations.

TABLE 4

| | | | | SEQ ID |
|---|---|---|---|---|
| ASO# | | Modified AS Seq | Length | NO: |
| 1 | 57 | +G*+C*+U*+U*G*C*A*G*G*T*G*C*A*+C*+G*+A*+U* | 17 | 30 |
| 2 | 4083 | +U*+G*+U*+C*G*A*T*G*C*A*G*T*A*+G*+G*+U*+C* | 17 | 31 |
| 3 | 4088 | +U*+G*+U*C*G*A*T*G*C*A*G*T*A*+G*+G*+U* | 16 | 32 |
| 4 | 4119 | +C*+C*+U*+C*G*T*T*G*T*C*G*A*T*+G*+C*+A*+G* | 17 | 33 |
| 5 | 4485 | +U*+U*+G*+A*G*G*T*C*C*C*C*G*T*+A*+G*+G*+U* | 17 | 34 |
| 6 | 6333 | +G*+C*+U*+C*G*T*C*T*A*C*C*T*C*+C*+U*+U*+C* | 17 | 35 |
| 7 | 6338 | +G*+C*+U*C*G*T*C*T*A*C*C*T*C*+C*+U*+U* | 16 | 36 |
| 8 | 6344 | +U*+G*+C*T*C*G*T*C*T*A*C*C*T*+C*+C*+U* | 16 | 37 |
| 9 | 7719 | +U*+C*+U*+C*G*T*C*C*A*T*G*C*C*+U*+U*+C*+G* | 17 | 38 |
| 10 | 7737 | +C*+C*+A*+U*C*T*C*G*T*C*C*A*T*+G*+C*+C*+U* | 17 | 39 |
| 11 | 8283 | +U*+C*+U*+U*C*G*A*A*C*T*C*G*C*+C*+C*+U*+C* | 17 | 40 |
| 12 | 8318 | +C*+C*+U*C*C*T*C*T*T*C*G*A*A*+C*+U*+C* | 16 | 41 |
| 13 | 10873 | +G*+G*+U*C*A*G*A*G*G*T*A*A*+G*+G*+C* | 15 | 42 |
| 14 | 525 | +U*+G*+C*+A*C*G*A*T*T*T*C*C*C*+G*+C*+A*+U* | 17 | 43 |
| 15 | 4095 | +G*+U*+U*+G*T*C*G*A*T*G*C*A*G*+U*+A*+G*+G* | 17 | 44 |
| 16 | 4109 | +C*+C*+U*+C*+G*T*T*G*T*C*G*A*T*G*C*+A*+G*+U*+A* | 19 | 45 |
| 17 | 4113 | +C*+U*+C*+G*T*T*G*T*C*G*A*T*G*+C*+A*+G*+U* | 17 | 46 |
| 18 | 6349 | +U*+G*+C*T*C*G*T*C*T*A*C*C*+U*+C*+C* | 15 | 47 |
| 19 | 7108 | +G*+U*+G*+C*T*G*T*T*G*C*C*G*A*T*+G*+A*+A*+G* | 18 | 48 |
| 20 | 7118 | +G*+U*+G*C*T*G*T*T*G*C*C*G*A*+U*+G*+A* | 16 | 49 |
| 21 | 7715 | +A*+U*+C*+U*+C*G*T*C*C*A*T*G*C*C*T*+U*+C*+G*+C* | 19 | 50 |
| 22 | 7732 | +C*+C*+A*+U*C*T*C*G*T*C*C*A*T*G*+C*+C*+U*+U* | 18 | 51 |
| 23 | 7743 | +U*+C*+C*+A*T*C*T*C*G*T*C*C*A*+U*+G*+C*+C* | 17 | 52 |
| 24 | 8276 | +U*+U*+C*G*A*A*C*T*C*G*C*C*C*+U*+C*+U* | 16 | 53 |
| 25 | 8289 | +C*+U*+C*+U*T*C*G*A*A*C*T*C*G*+C*+C*+C*+U* | 17 | 54 |
| 4A | 4119 | +C*+C+U+CG*T*T*G*T*(5′Me)C*G*A*T*+G+C+A+G | 17 | 55 |
| 4B | 4119 | +C*+C+U+CG*T*T*G*T*(5′Me)C*G*A*T*+G+C+A*+G | 17 | 56 |
| 6A | 6333 | +G*+C+U+CG*T*(5′Me)C*T*A*(5′Me)C*(5′Me)C*T*C*+C+U+ | 17 | 57 |
| | | U+C | | |
| 6B | 6333 | +G*+C+U+CG*T*(5′Me)C*T*A*(5′Me)C*(5′Me)C*T*C*+C+U+ | 17 | 58 |
| | | U* +C | | |
| 7A | 6338 | +G*+C+U+CG*T*(5′Me)C*T*A*(5′Me)C*(5′Me)C*T*(5′Me)C+C | 16 | 59 |
| | | +U+U | | |
| 7B | 6338 | +G*+C+U+CG*T*(5′Me)C*T*A*(5′Me)C*(5′Me)C*T*(5′Me)C+C | 16 | 60 |
| | | +U* +U | | |
| NTC | 158 | +U*+A+C+G(5′Me)C*G*A*C*U*U*A*U*+G+C+G*+G | 16 | 61 |

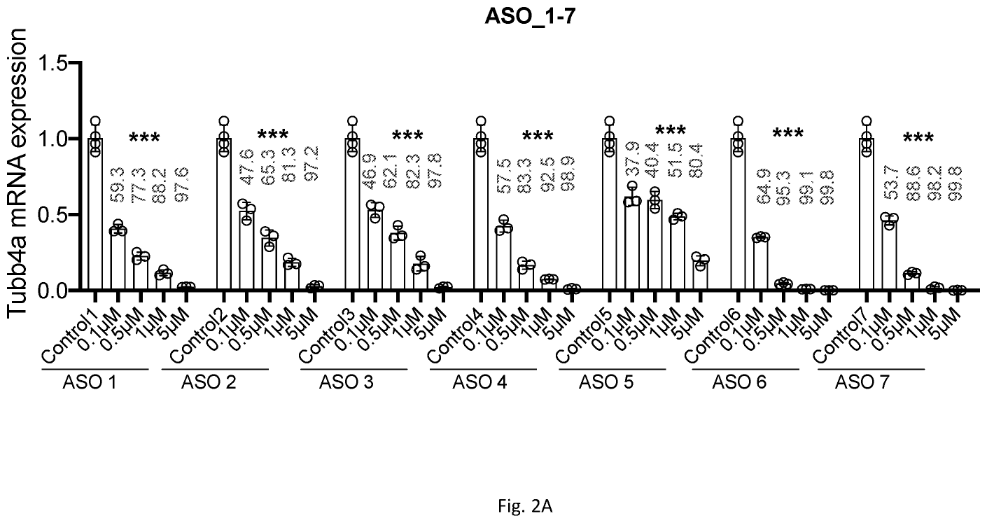

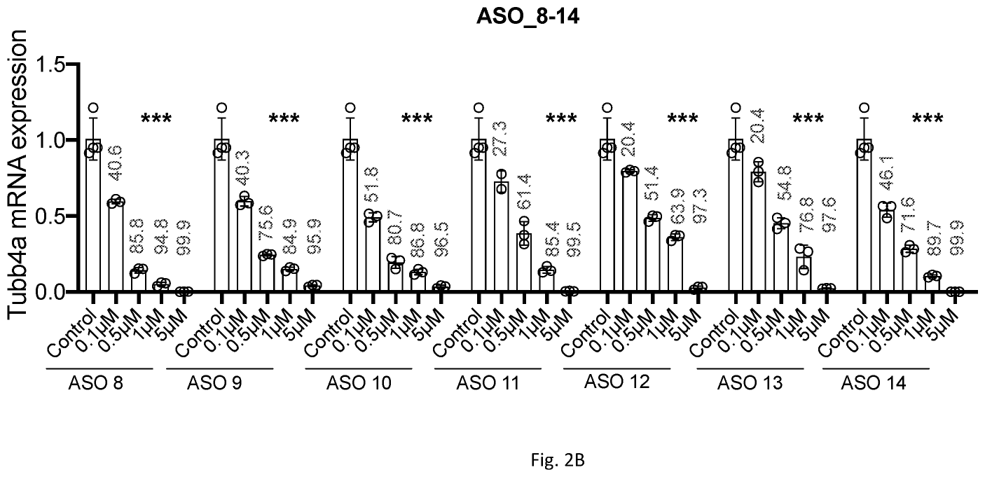

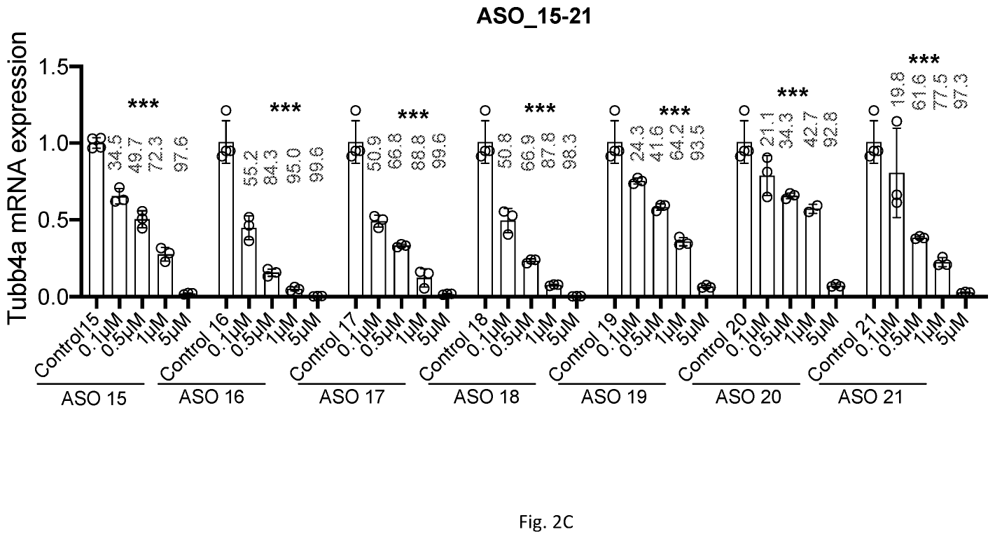

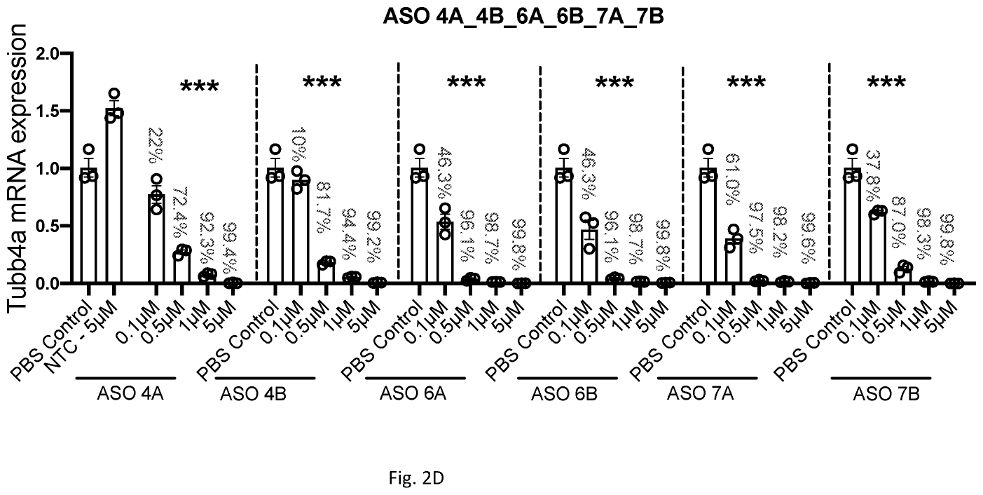

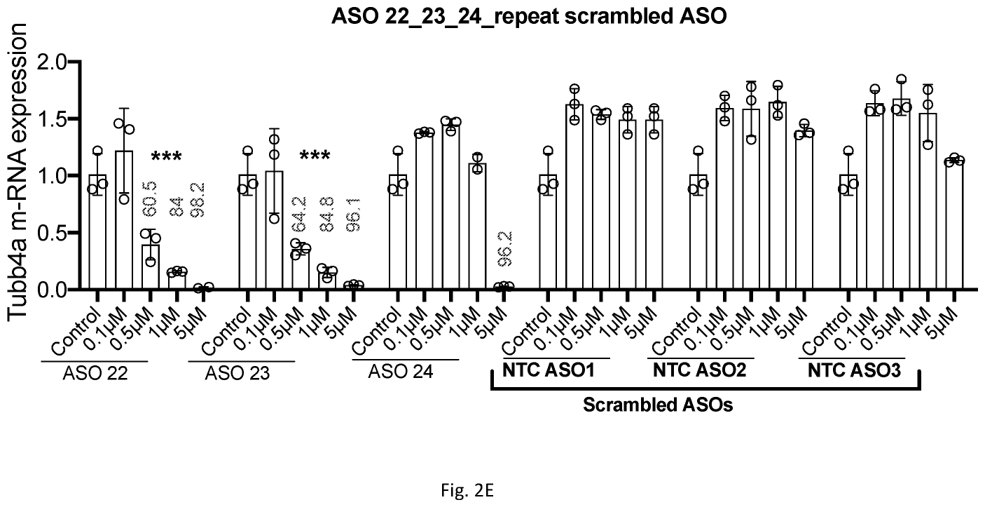

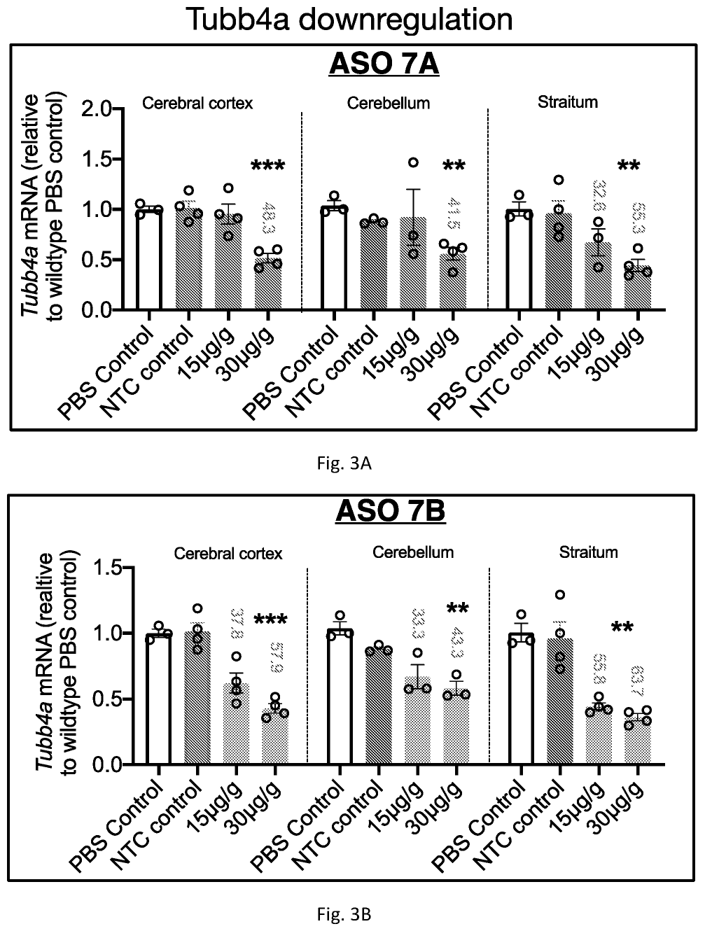

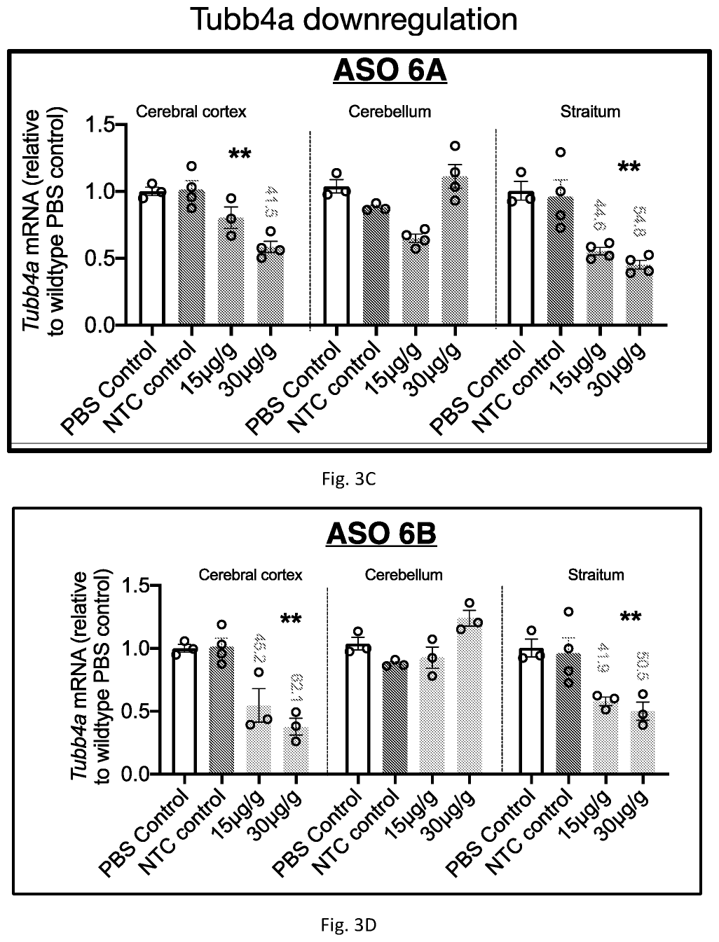

Concomitantly, we screened mouse ASO sequences in vitro on murine cell line and ASO sequences are listed in Table below. See Table 4 and FIG. 2A-2D.

The listed ASO sequences were tested in vivo in mice using single intracerebroventricular (ICV) bolus injection route of these different ASO sequences into wild-type mice can be employed to identify those ASOs which maximally reduce TUBB4A levels. This approach can also be employed in TUBB4A^{D249N/D249N} mice to ameliorate symptoms of H-ABC leukodystrophy.

### Example II

### Analysis of Antisense Oligonucleotide Mediated Therapeutic Suppression of TUBB4A and Toxicity in Humans Cells

By screening human TUBB4A ASOs in vitro in human cell lines and using the ASO sequences described in Example I, new antisense oligonucleotides (ASO) which are targeted against, and reduce human TUBB4A were developed. See Table 5.

These ASO gapmers include three different modifications—
1. Flanking 5′ and 3′ end sequences are 2-O-(2-Methoxyethyl)-oligori-bonucleotides indicated as NO
2. Flanking 5′ and 3′ end sequences are locked nucleic acids indicated as
3. Flanking 5′ and 3′ end sequences include mixed backbone of locked nucleic acids and 2′-O-(2-Methoxyethyl)-oligori-bonucleotides.

TABLE 5
Additional Human ASOs

| | | Gapmer | # PTO | SEQ ID |
|---|---|---|---|---|
| ASO | ASO design | type | bonds | NO: |
| H2-1 | +U*+G*+C^{m}*a*c^{m}*g*c^{m}*t*c^{m}*a*g*c^{m}*a*+U*+C^{m}*+U | 3-10-3 | 15 | 62 |
| H2-2 | +U*@G*+C^{m}*a*c^{m}*g*c^{m}*t*c^{m}*a*g*c^{m}*a*+U*@C^{m}*+U | 3-10-3 | 15 | 63 |
| H2-3 | @U*@G*@C^{m}*a*c^{m}*g*c^{m}*t*c^{m}*a*g*c^{m}*a*@U*@C^{m}* | 3-10-3 | 15 | 64 |
| | @U | | | |
| H2-4 | +C^{m}*+U*+G*+C^{m}*a*c^{m}*g*c^{m}*t*c^{m}*a*g*c^{m}*a*+U*+C^{m} | 4-10-4 | 17 | 65 |
| | *+U*+G | | | |
| H10-1 | +G*+G*+A*c^{m}*t*t*g*c^{m}*a*g*g*t*g*+U*+A*+G | 3-10-3 | 15 | 66 |
| H10-2 | @G*@G@A*c^{m}*t*t*g*c^{m}*a*g*g*t*g*@U@A*@G | 3-10-3 | 13 | 67 |
| H10-3 | +A*+G+G+A*c^{m}*t*t*g*c^{m}*a*g*g*t*g*+U+A+G*+A | 4-10-4 | 13 | 68 |
| H10-4 | @G@G@A*c^{m}*t*t*g*c^{m}*a*g*g*t*g*@U@A@G | 3-10-3 | 10 | 69 |
| H11-1 | +G*+A*+A*c^{m}*t*g*c^{m}*a*g*c^{m}*t*c^{m}*+G*+G+A | 3-9-3 | 14 | 70 |
| H11-2 | @G*+A*+A*c^{m}*t*g*c^{m}*a*g*c^{m}*t*c^{m}*+G*+G*@A | 3-9-3 | 14 | 71 |
| H11-3 | @U*+G*+A*+A*c^{m}*t*g*c^{m}*a*g*c^{m}*+G*+G*+A* | 4-9-4 | 16 | 72 |
| | @G | | | |
| H14-1 | +G+U+A+G*g*t*c^{m}*t*c^{m}*a*t*c^{m}*c^{m}*+G+U+A+U | 4-9-4 | 9 | 73 |
| H15-1 | +G*+G*+U*c^{m}*a*g*a*a*g*c^{m}*c^{m}*t*+C^{m}*+G*+A | 3-9-3 | 14 | 74 |
| H15-2 | @G*@G*+U*c^{m}*a*g*a*a*g*c^{m}*c^{m}*t*+C^{m}*@G*@A | 3-9-3 | 14 | 75 |
| H15-3 | +A*+A*+G*+G*+U*c^{m}*a*g*a*a*g*c^{m}*c^{m}*t*+C^{m}*+G*+ | 5-9-5 | 19 | 76 |
| | A*+G*+G | | | |
| H15-4 | +G+G+UC^{m}*A*G*A*A*G*C^{m}*C^{m}*T*+C^{m}+G+A | 3-9-3 | 9 | 77 |
| H22 | @G@C^{m}@Ac^{m}*g*c^{m}*t*c^{m}*a*g*c^{m}*a*t*@C^{m}@T@G | 3-10-3 | 10 | 78 |
| H23 | @C^{m}@T@C^{m}@T@Gc^{m}*a*c^{m}*g*c^{m}*t*c^{m}*a*g*@C^{m}@A@T | 5-9-5 | 9 | 79 |
| | @C^{m}@T | | | |
| H24 | @G@C^{m}@Ac^{m}*g*c^{m}*t*c^{m}*a*g*c^{m}*a*@T@C^{m}@T | 3-9-3 | 9 | 80 |
| H25 | @T@C^{m}@T@Gc^{m}*a*c^{m}*g*c^{m}*t*c*a*g*c^{m}*@A@T@C^{m} | 4-10-3 | 10 | 81 |
| H26 | @C^{m}@T@Gc^{m}*a*c^{m}*g*c^{m}*t*c^{m}*a*g*@C^{m}@A@T@C^{m} | 3-9-4 | 9 | 82 |
| H27 | @G@G@Tc^{m}*a*g*a*a*g*c^{m}*c^{m}*t*@C^{m}@G@A | 3-9-3 | 9 | 83 |
| H28 | @G@G@T@C^{m}a*g*a*a*g*c^{m}*c^{m}*t*@C^{m}@G@A | 4-8-3 | 8 | 84 |
| H29 | @G@G@Ac^{m}*t*t*g*c^{m}*a*g*g*t*g*@T@A@G | 3-10-3 | 10 | 85 |
| H30 | +A+G+G+Ac^{m}*t*t*g*c^{m}*a*g*g*t*+G+U+A+G | 4-9-4 | 9 | 86 |
| H31 | @A+G@G+Ac^{m}*t*t*g*c^{m}*a*g*g*+U@G+U@A | 4-8-4 | 8 | 87 |
| H32 | +G+G+Ac^{m}*t*t*g*c^{m}*a*g*g*t^{m}*+G+U+A | 3-9-3 | 9 | 88 |
| H33 | +A+G+Ga*c^{m}*t*t*g*c^{m}*a*g*g*+U+G+U | 3-9-3 | 9 | 89 |
| H34 | @G@A@Ac^{m}*t*g*c*a*g*c^{m}*t*c^{m}*g*@G@A@G | 3-10-3 | 10 | 90 |
| H35 | @G@A@Ac^{m}*t*g*c^{m}*a*g*c^{m}*t*c^{m}*@G@G@A | 3-9-3 | 9 | 91 |
| H36 | @A@A@Gg*t*g*a*a*c*t*g*c^{m}*@A@G@C^{m} | 3-9-3 | 9 | 92 |
| ASO | +G+C^{m}+G+C^{m}+At*c^{m}*a*a*a*g*g*t*c^{m}*a*+G+A+A+G+C^{m} | 5-10-5 | 10 | 93 |
| 1477-1 | | | | |
| ASO | +C^{m}+C^{m}+U+G+Gg*a*a*t*g*t*cm*a*a*g*+G+U+U+G+G | 5-10-5 | 10 | 94 |
| 1659-2 | | | | |

*phosphorothioate linkage
+sign-2′-O-(2-Methoxyethyl)-oligori-bonucleotides (2′-MOE)
@-locked nucleic acid (LNA)
^{m}-5′methyl

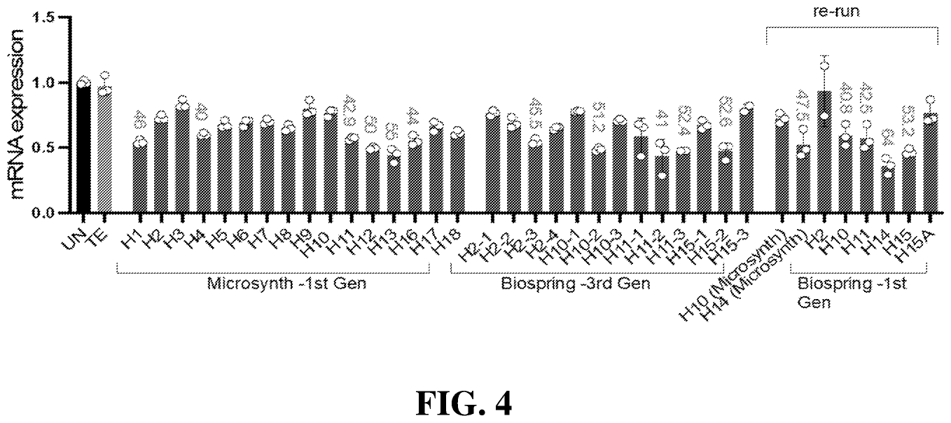

The library of ASOs from Table 4 and Table 5 (ASO H2-1 to H15-4) were synthesized a Biospring company and was screened in SHTYSY5Y cells using gymnosis. The cells were plated at 250,000 cells per well and treated with 10 m of ASO for 96 hours. The RNA was then extracted and subjected to qPCR to assess the TUBB4A expression. The results are shown in FIG. 4.

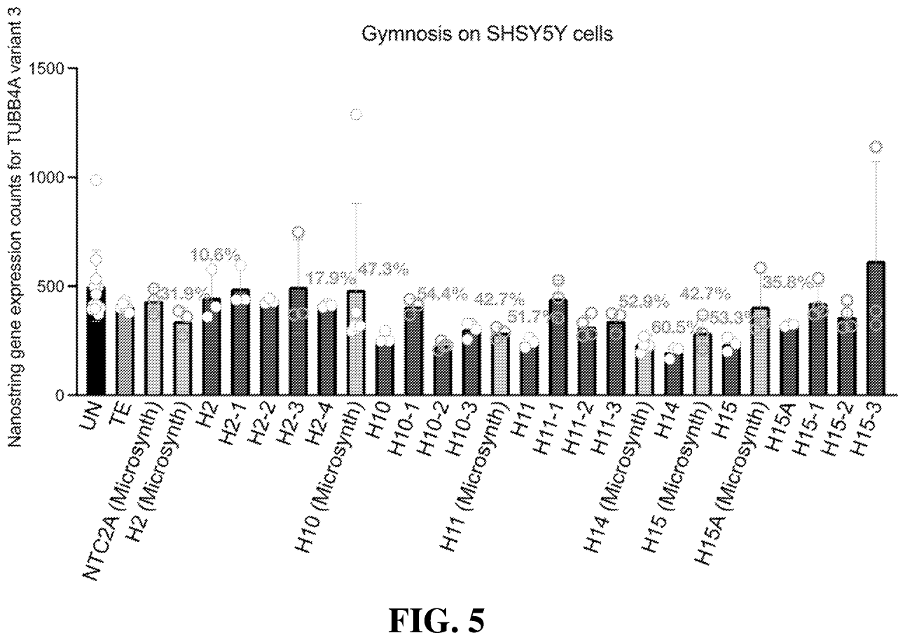

The library of ASOs was further screened in SHTYSY5Y cells using gymnosis. The cells were plated at 250,000 cells per well and treated with 10 m of ASO for 96 hours. The RNA was then extracted to conduct Nanostring for testing the levels of TUBB4A expression. The results are shown in FIG. 5.

The results from FIGS. 4 and 5 indicate that ASO H14 and H15 is particularly well suited for downregulation of TUBB4A. This data further indicates that ASO H14 and H15 is well suited for use as a model design for follow on generation of additional ASOs. Additional targets particularly well suited for downregulation of TUBB4A and generation of optimized ASOs, include ASO H13, H16, and H1.

After downregulation of TUBB4A was confirmed for the ASOs of Tables 3 and 5, these ASOs were tested for in vitro toxicity using the ApoTox-Glo Triplex kit. This kit combines three assay chemistries to assess cellular viability, cytotoxicity, and apoptosis within a single assay well. The first part of this assay involves simultaneously measuring two protease activities, one as a marker for cell viability and the other as a marker for cytotoxicity. The cells are then exposed to a second fluorogenic cell-impermeant peptide substrate to measure dead-cell protease activity to determine lost membrane integrity, thereby measuring the levels of apoptosis from the ASO.

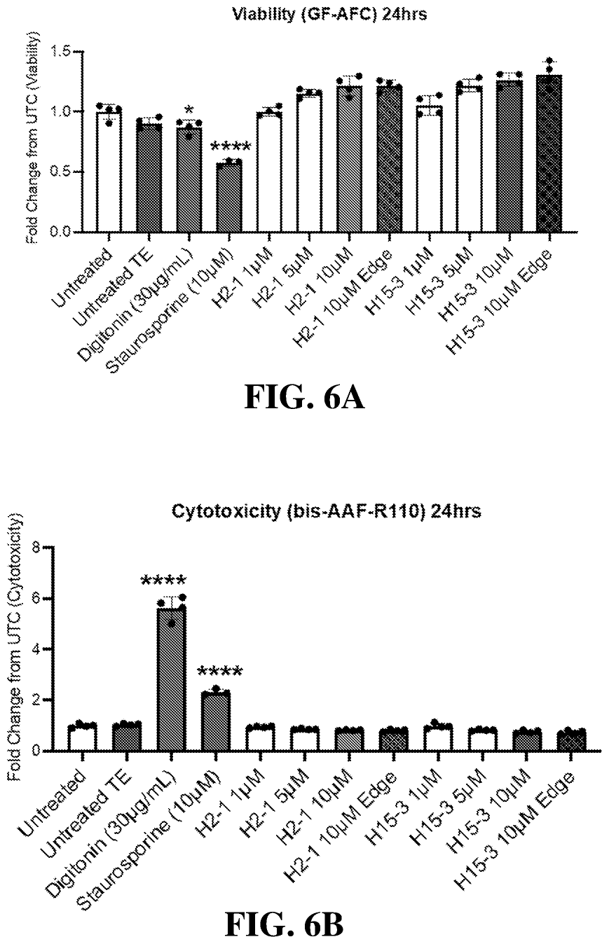

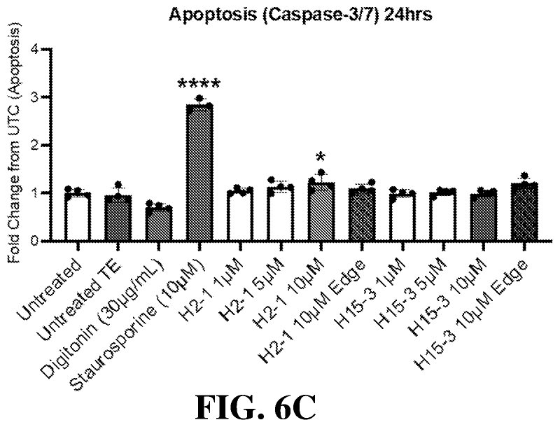

Initially, two ASOs were selected to detect the in vitro toxicity of ASOs 24 hours after administration. SHYSY5Y cells were plated at 10,000 cells per well and treated with 10 μm of ASO for 24 hours. As a positive control, two wells were treated with 10 m Digitonin or Staurosporine. Cell viability, cytotoxicity and apoptosis were then tested for each well. The results are shown in FIGS. 6A-6C. The tested ASOs showed no toxicity in vitro.

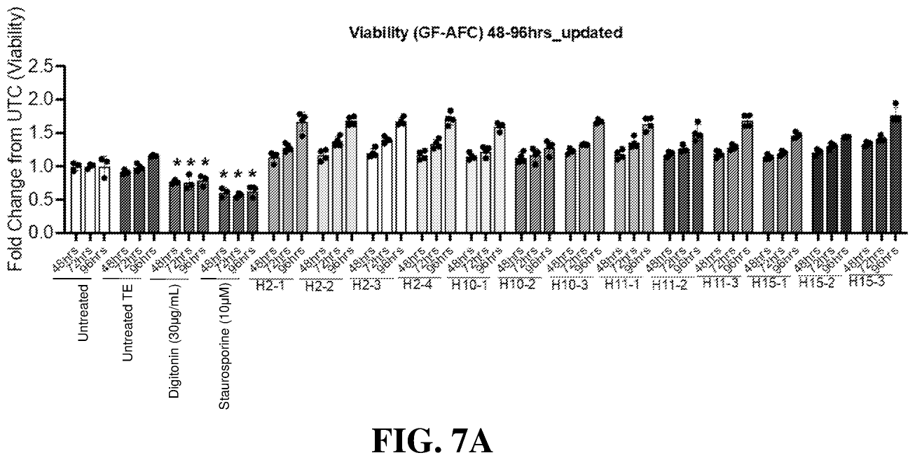

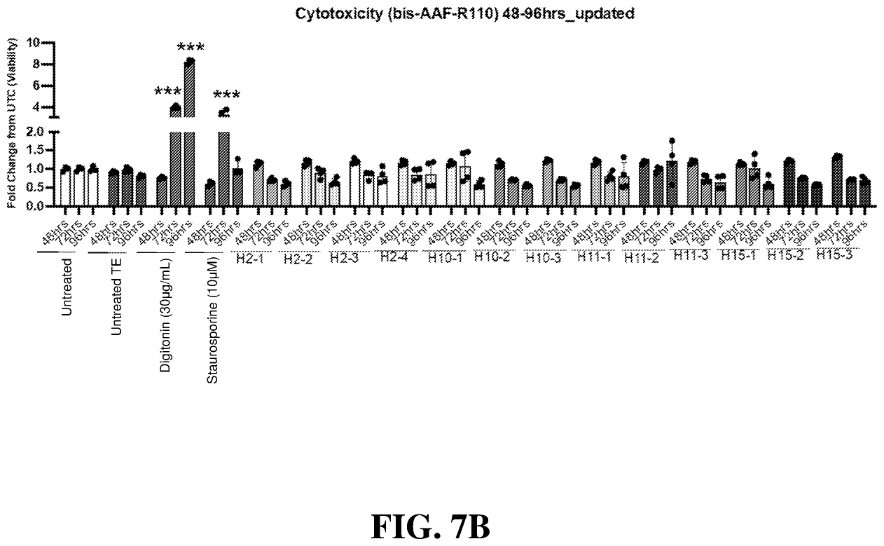

After confirmation of the preliminary results, additional ASOs were tested for in vitro toxicity. SHYSY5Y cells were plated at 10,000 cells per well and treated with 10 μm of ASO for 48, 72, or 96 hours. As a positive control, wells were treated with 10 μm Digitonin or Staurosporine for 48, 72, or 96 hours. Additionally, untreated SHYSY5Y cells and untreated vehicle were tested after 48, 72, or 96 hours. The results are shown in FIGS. 7A-7C. Again, the tested ASOs showed no toxicity in vitro.

Based on the TUBB4A downregulation and minimal toxicity in vitro, ASOs H10-2, H14, and H15 were tested on human iPSC derived Medium spiny neurons (HiPSC-MSNs). These ASOs were selected as they showed consistent TUBB4A downregulation by both qRT-PCR and Nanostring assays. The results in FIG. 4 shows that H10-2 had 51.2% knockdown of TUBB4A in SH-SY4Y cells while FIG. 5 shows that H10-2 had 54.4% knockdown of TUBB4A in SH-SY4Y cells. The results in FIG. 4 demonstrate that H14 had 64.0% knockdown of TUBB4A in SH-SY4Y cells, while FIG. 5 shows that H14 had 60.5% knockdown of TUBB4A in SH-SY4Y cells. The results in FIG. 4 shows that H15 had 53.2% knockdown of TUBB4A in SH-SY4Y cells while FIG. 5 shows that H15 had 53.3% knockdown of TUBB4A in SH-SY4Y cells.

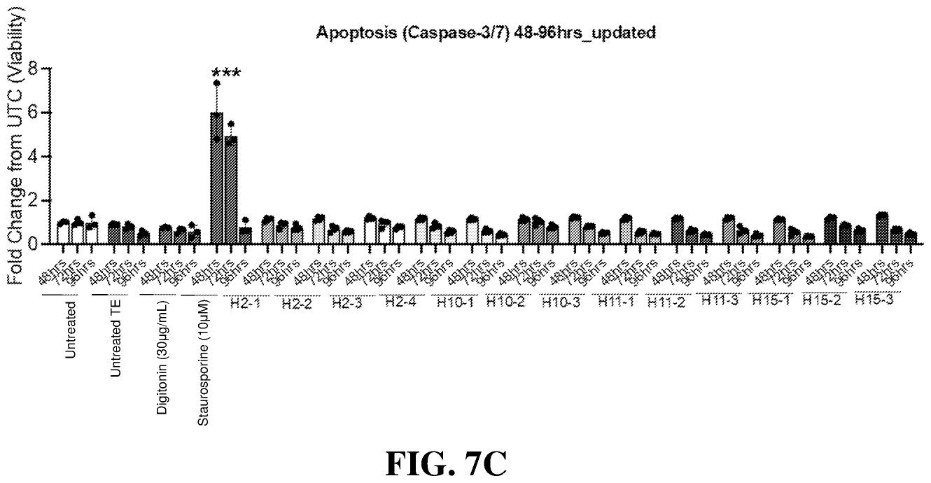

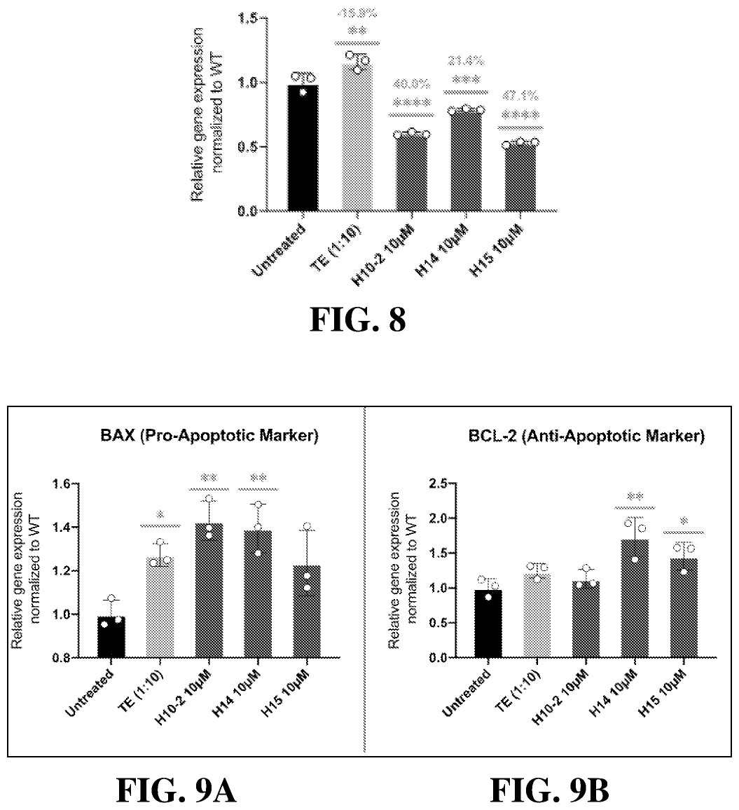

TUBB4A gene expression in was tested in HiPSC-MSNs using Gymnosis. The HiPSC-MSNs were treated with the ASO candidates at day 37. After four days, the cells were retreated with the ASOs. On day 7, the RNA was extracted and TUBB4A expression was determined using qRT-PCR. The results are shown in FIG. 8.

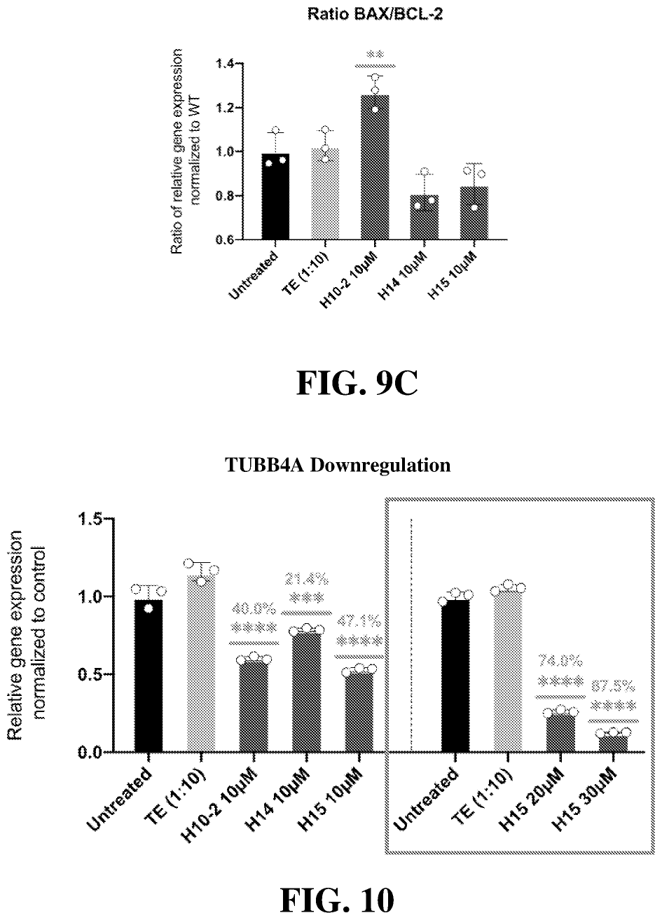

After downregulation of TUBB4A was confirmed for the ASOs in HiPSC-MSNs, the ASOs were tested for toxicity in HiPSC-MSNs. The HiPSC-MSNs were treated with the ASO candidates at day 37. After four days, the cells were retreated with the ASOs. On day 7, the RNA was extracted and BAX and BCL-2 expression was determined using qRT-PCR. The BCL-2 oncoprotein regulates programmed cell death by providing a survival advantage to rapidly proliferating cells, and the BAXprotein promotes apoptosis by enhancing cell susceptibility to apoptotic stimuli. Due to these functions, the BAX and BCL-2 proteins are commonly used as indicators of cellular toxicity. Ratios of BAXBCL-2 of >1 indicates more apoptosis, whereas ratios <1 indicate less apoptosis. The results of this experiment are shown in FIG. 9A-C.

The results of this Experiment show that the tested ASOs are effective at downmodulating TUBB4A and non-toxic to the treated cells.

### Example III

### Dose Response Analysis of Control iPS-MSNs for Dose Determination of Antisense Oligonucleotides

After determining that the ASOs described above are safe and effective for the downmodulation of TUBB4A, additional experimentation was performed to identify the appropriate dosage of the ASO. The dose response of the ASO candidates was performed on control HiPS-MSNs using gymnosis where the oligo is suspended in a biologically compatible buffer, including without limitation phosphate buffered saline, and CSF mimicking buffer. The HiPSC-MSNs were treated with 10 μm, 20 μm, and 30 μm ASO H15 at day 37. The HiPSC-MSNs were then retreated after four days using the same dosage. Seven days from the initial treatment, the RNA was extracted and TUBB4A expression was determined using qRT-PCR. The results of this experiment are shown in FIG. 10. As the dose of ASO increased, relative gene expression decreased.

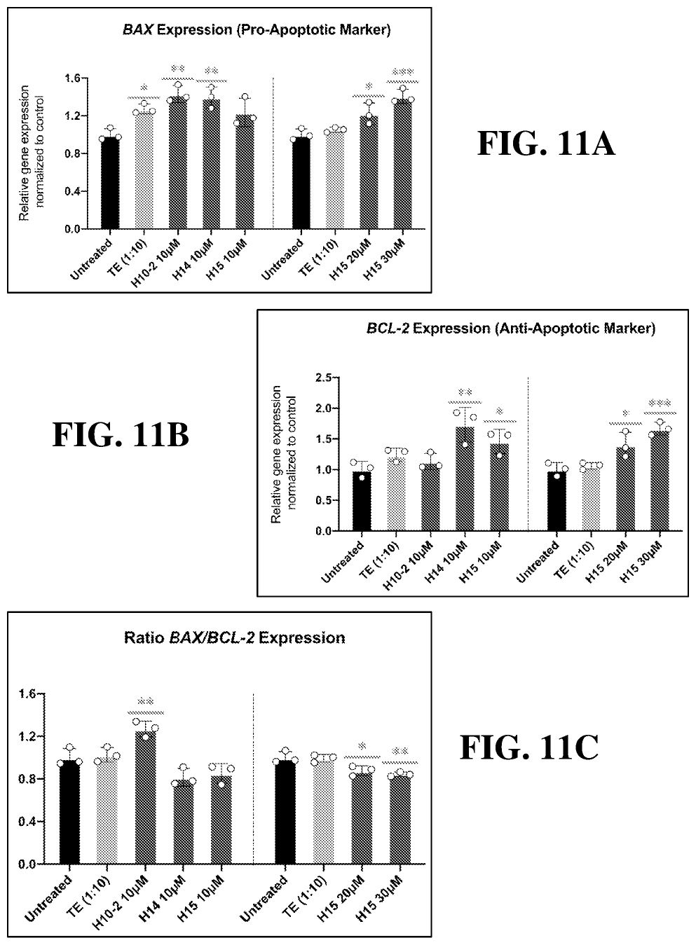

After showing increased downregulation of TUBB4A with increasing dosage, the toxicity of the ASO was determined in HiPSC-MSNs. The HiPSC-MSNs were treated with 10 μm, 20 μm, and 30 μm ASO H15 at day 37. The HiPSC-MSNs were then retreated after four days using the same dosage. Seven days from the initial treatment, the RNA was extracted and BAX and BCL-2 expression was determined using qRT-PCR. Ratios of BAXBCL-2 of >1 indicates more apoptosis, whereas ratios <1 indicate less apoptosis. The results of this experiment are shown in FIGS. 11A-11C.

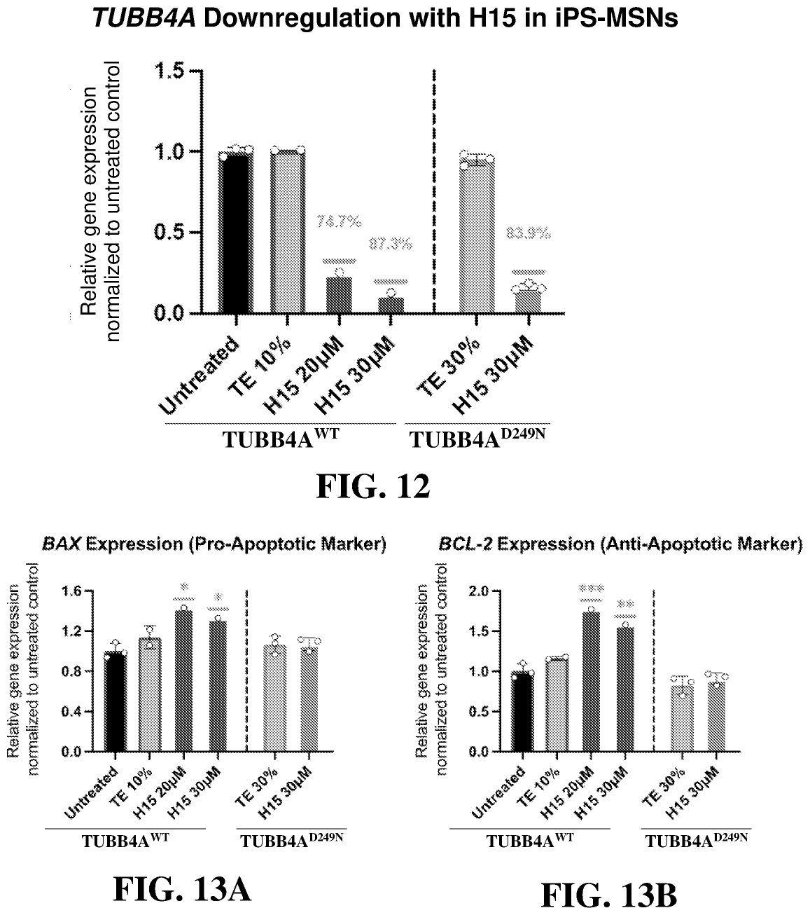

TUBB4A downregulation by ASO H15 was further analyzed using mutant HiPSC-MSNs (TUBB4A^{D249N}) using gymnosis. The mutant HiPSC-MSNs were treated with 30 μm ASO H15 at day 37. Wild type HiPSC-MSNs cells were also rerun to confirm the previous downregulation results by treating the cells with 10 μm, 20 μm, and 30 μm ASO H15 at day 37. The mutant and wild type HiPSC-MSNs were then retreated after four days using the same dosage. Seven days from the initial treatment RNA was extracted and TUBB4A expression was determined using qRT-PCR. ASO H15 showed similar downregulation of TUBB4A in both mutant and wild type cells. The results of this experiment are shown in FIG. 12.

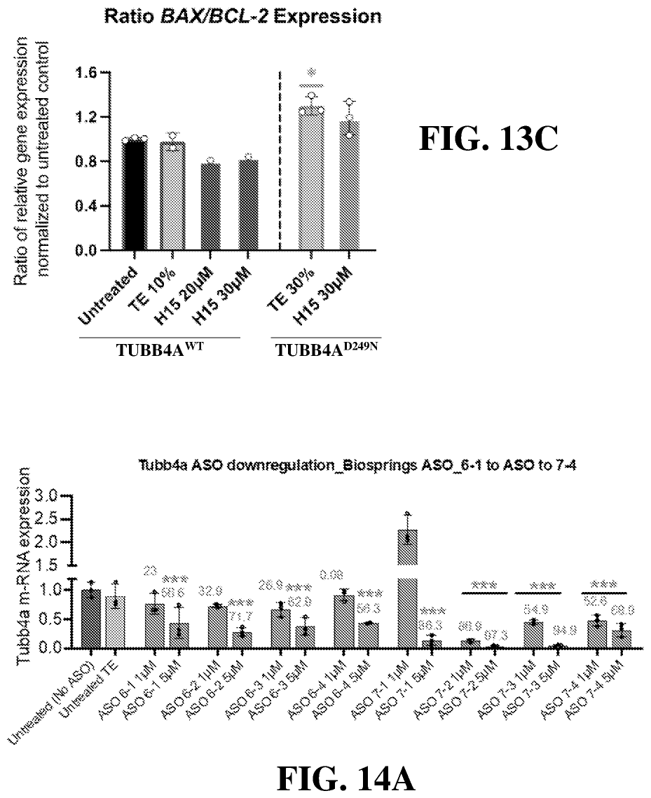

After showing increased downregulation of TUBB4A in mutant HiPS-MSNs, the toxicity of the ASO was confirmed in the mutant cells. The mutant HiPSC-MSNs were treated with 30 μm ASO H15 at day 37. Wild type HiPSC-MSNs cells were also rerun to confirm the previous downregulation results by treating the cells with 10 μm, 20 μm, and 30 μm ASO H15 at day 37. The mutant and wild type HiPSC-MSNs were then retreated after four days using the same dosage. Seven days from the initial treatment RNA was extracted and BAX and BCL-2 expression was determined using qRT-PCR. The results of this experiment are shown in FIGS. 13A-13C.

### Example IV

### Analysis of Antisense Oligonucleotide Mediated Therapeutic Suppression of Tubb4a and Toxicity in Mouse Cells

By screening TUBB4A ASOs in vitro murine cell lines and using the ASO sequences described in Example I, additional antisense oligonucleotides (ASO) which are targeted against and reduce murine TUBB4A were developed. See Table 6.

TABLE 6
Additional Mouse ASOs

| | | Gapmer | # PTO | SEQ ID |
|---|---|---|---|---|
| ASO | ASO design | type | bonds | NO: |
| 6-1 | +G*+C^{m}*+U*+C^{m}*g*t*c^{m}*t*a*c^{m}*c^{m}*t*c^{m}*+C^{m}*+U*+U* | 4-9-4 | 16 | 95 |
| | +C^{m} | | | |
| 6-2 | +G*+C^{m}+U+C^{m}*g*t*c^{m}*t*a*c^{m}*c^{m}*t*c^{m}*+C^{m}+ U+U*+C^{m} | 4-9-4 | 12 | 96 |
| 6-3 | +U*+G*+C^{m}*+U*+C^{m}*g*t*c^{m}*t*a*c^{m}*c^{m}*t*c^{m}*+C^{m}*+U* | 5-9-5 | 18 | 97 |
| | +U*+C^{m}*+A | | | |
| 6-4 | +U*+G+C^{m}+U+C^{m}*g*t*c^{m}*t*a*c^{m}*c^{m}*t*c^{m}*C^{m}+U+U+C^{m} | 5-9-5 | 12 | 98 |
| | *+A | | | |
| 6-5 | +G+C^{m}+U+C^{m}*g*t*c^{m}*t*a*c^{m}*c^{m}*t*c^{m}*+C^{m}+U+U+C^{m} | 4-9-4 | 10 | 99 |
| 7-1 | +G*+C^{m}*+U*c^{m}*g*t*c^{m}*t*a*c^{m}*c^{m}*t*c^{m}*+c^{m}*+U*+U | 3-10-3 | 15 | 100 |
| 7-2 | +G*@C^{m}*+U*c^{m}*g*t*c^{m}*t*a*c^{m}*c^{m}*t*c^{m}*+C^{m}*@U*+U | 3-10-3 | 15 | 101 |
| 7-3 | +U*+G*+C^{m}*+U*c^{m}*g*t*c^{m}*t*a*c^{m}*c^{m}*t*c^{m}*+C^{m}*+U*+ | 4-10-4 | 17 | 102 |
| | U*+C^{m} | | | |
| 7-4 | +U*+G+C^{m}+U*c^{m}*g*t*c^{m}*t*a*c^{m}*c^{m}*t*c^{m}*+C^{m}+U+U*+C^{m} | 4-10-4 | 13 | 103 |
| 7-5 | +G+C^{m}+Uc^{m}*g*t*c^{m}*t*a*c^{m}*c^{m}*t*c^{m}*+C^{m}+U+U | 3-10-3 | 10 | 104 |
| 7-6 | +U+G+C^{m}+Uc^{m}*g*t*c^{m}*t*a*c^{m}*c^{m}*t*c^{m}*+C^{m}+U+U+C^{m} | 4-10-4 | 10 | 105 |
| 8-1 | +U*+G*+C^{m}*t*c^{m}*g*t*c^{m}*t*a*c^{m}*c^{m}*t*+C^{m}*+C^{m}*+U | 3-10-3 | 15 | 106 |
| 8-2 | +U*@G+C^{m}*t*c^{m}*g*t*c^{m}*t*a*c^{m}*c^{m}*t*+C^{m}@C^{m}*U | 3-10-3 | 13 | 107 |
| 8-3** | +U*+C^{m}*+U*+G*+C^{m}*t*c^{m}*g*t*c^{m}*t*a*c^{m}*c^{m}*t*+C^{m}*+C^{m} | 5-10-5 | 19 | 108 |
| | *+U*+U*+C^{m} | | | |
| 8-4** | +U*+C^{m}+U+G+C^{m}*t*c^{m}*g*t*c^{m}*t*a*c^{m}*c^{m}*t*+C^{m}+C^{m}+ | 5-10-5 | 13 | 109 |
| | U+U*+C^{m} | | | |
| 8-5 | +U+G+C^{m}t*c^{m}*g*t*c^{m}*t*a*c^{m}*c^{m}*t*+C^{m}+C^{m}+U | 3-10-3 | 10 | 110 |
| 18-1 | +U*+G*+C^{m}*t*c^{m}*g*t*c^{m}*t*a*c^{m}*c^{m}*+U*+C^{m}*+C^{m} | 3-10-3 | 14 | 111 |
| 18-2 | @U*@G*@C^{m}*t*c^{m}*g*t*c^{m}*t*a*c^{m}*c^{m}*@U*@C^{m}*@C^{m} | 3-10-3 | 14 | 112 |
| 18-3 | +C^{m}*+U*+G*+C^{m}*t*c^{m}*g*t*c^{m}*t*a*c^{m}*c^{m}*+U*+C^{m}*+C^{m} | 4-9-4 | 16 | 113 |
| | *+U | | | |
| 18-5 | +U+G+C^{m}t*c^{m}*g*t*c^{m}*t*a*c^{m}*c^{m}*+U+C^{m}+C^{m} | 3-9-3 | 9 | 114 |

*phosphorothioate linkage
+sign-2′-O-(2-Methoxyethyl)-oligori-bonucleotides (2′-MOE)
@-locked nucleic acid (LNA)
^{m}-5′methyl

Mouse ASOs lack the homology with the human TUBB4A. That is why we have re-designed concomitant human ASO sequences which are screened separately. We will establish proof-of-concept with the mouse ASOs and human ASOs will be translated to the clinical trial.

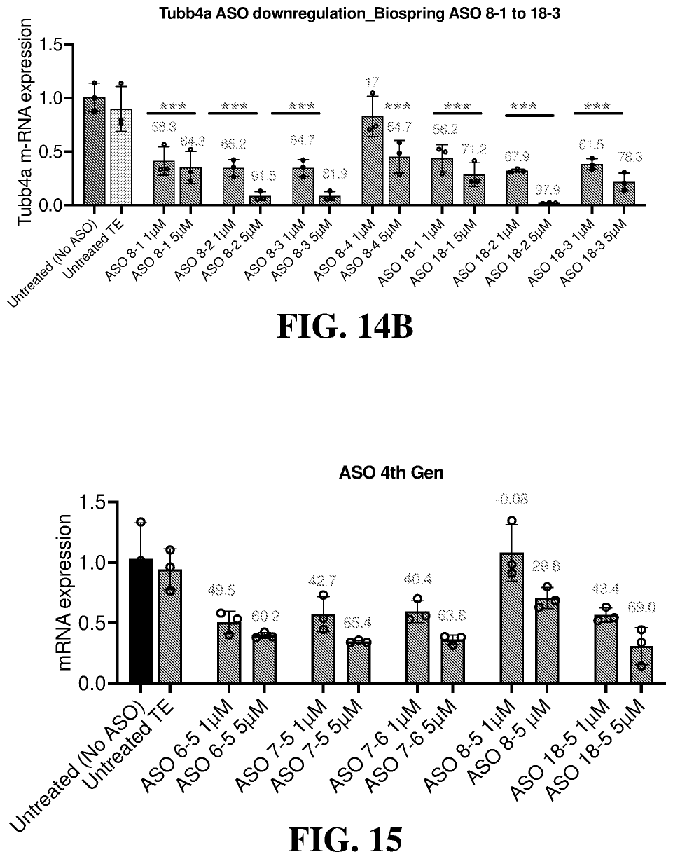

The library ASOs was screened in cortical mouse primary neurons cells using gymnosis. The cells were plated at 200,000 or 150,000 cells per well and treated with 1 μm and 5 μm of ASO for one week. The RNA was then extracted and subjected to qPCR to assess the TUBB4A expression. The results of these assays are shown in FIGS. 14A-14B and 15.

After downregulation of TUBB4A was confirmed for the ASOs of Tables 4 and 6, these ASOs were tested for in vitro toxicity using the ApoTox-Glo Triplex kit. This kit combines three assay chemistries to assess cellular viability and apoptosis within a single assay well. The first part of this assay involves simultaneously measuring a protease activity as a marker for cell viability. The cells are then exposed to a second fluorogenic cell-impermeant peptide substrate to measure dead-cell protease activity to determine lost membrane integrity, thereby measuring the levels of apoptosis from the ASO.

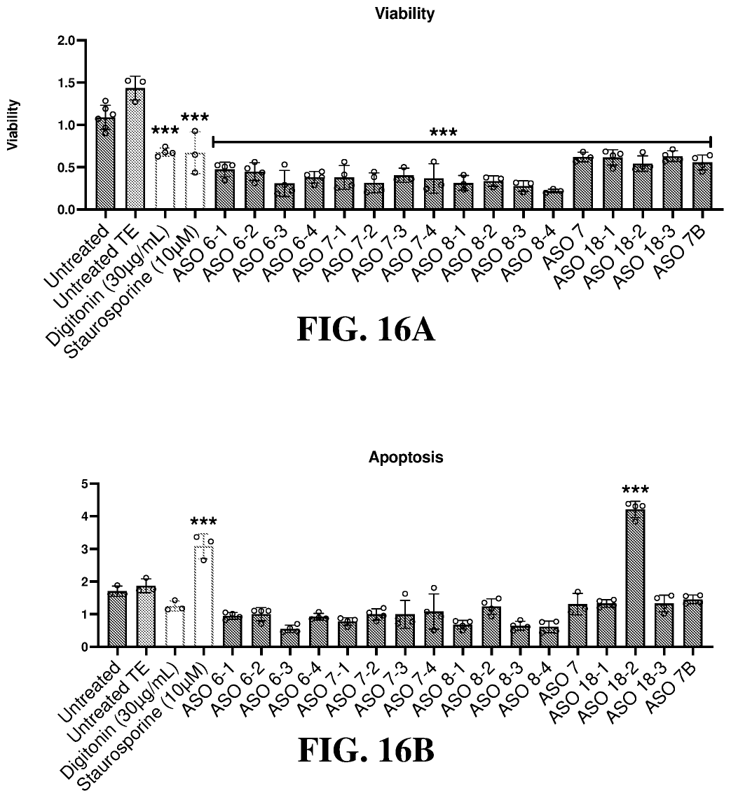

Several ASOs were screened to detect the in vitro toxicity of ASOs 96 hours after administration. Mouse cortical neurons cells were plated at 20,000 cells per well and treated with 5 μm of ASO for 96 hours. As a positive control, two wells were treated with 10 μm Digitonin or Staurosporine. Cell viability and apoptosis were then tested for each well. The results are shown in FIGS. 16A-16B. The tested ASOs showed no toxicity in vitro.

### Example V

### Analysis of Treatment of Wild Type Mice with Antisense Oligonucleotide

After analysis of the screening assays performed in Examples I and IV, ASOs 7-2, 7-3, 8-2, 8-3, 18-1, and 18-3 were chosen for analysis of ASO efficiency and toxicity in wild type mice. 150 μg/μL solutions containing 500 nmoles ASO were prepared.

The 30 adult mice aged P60 received an intracerebroventricular (ICV) injection with 15 μg/g or 30 μg/g ASO. Table 7 describes the observations of each WT mice.

TABLE 7
Status of Mice 31-28 days after injection

| Name | Treatment | Sex | Observations |
|---|---|---|---|
| Mouse1 | PBS | M | Active, Normal Gait |
| Mouse2 | ASO7-2 (15 μg/g) | M | Active, Dystonia |
| Mouse7 | ASO7-3 (15 μg/g) | M | Active, Normal Gait |
| Mouse8 | ASO7-3 (15 μg/g) | M | Active, Normal Gait, small mass present |
| Mouse9 | ASO7-3 (15 μg/g) | F | Active, Normal Gait |
| Mouse13 | ASO 8-2 (15 μg/g) | F | Active, Mild Dystonia, Loss of Hindlimb tone, |
| | | | Loss of Body tone, Ataxia |
| Mouse17 | ASO 8-3 (15 μg/g) | M | Hyperactive, Normal Gait |
| Mouse18 | ASO 8-3 (15 μg/g) | F | Hyperactive, Normal Gait |
| Mouse19 | ASO 8-3 (15 μg/g) | F | Hyperactive, Normal Gait |
| Mouse20 | ASO 8-3 (30 μg/g) | F | Active, Ataxia |
| Mouse21 | ASO 8-3 (30 μg/g) | M | Active, Ataxia |
| Mouse22 | ASO 18-1(15 μg/g) | F | Active, Normal Gait |
| Mouse23 | ASO 18-1(15 μg/g) | M | Active, Normal Gait |
| Mouse24 | ASO 18-1(30 μg/g) | M | Active, Mild dystonia, Loss of Hindlimb tone, |
| | | | Loss of Body tone |
| Mouse25 | ASO 18-1(30 μg/g) | F | Active, Mild dystonia, Loss of Hindlimb tone, |
| | | | Loss of Body tone |
| Mouse26 | ASO 18-3(15 μg/g) | F | Active, Normal Gait |
| Mouse27 | ASO 18-3(15 μg/g) | M | Active, Normal Gait |
| Mouse28 | ASO 18-3(15 μg/g) | F | Active, Normal Gait |
| Mouse29 | ASO 18-3(30 μg/g) | F | Active, Mild dystonia |
| Mouse30 | Non-injected | M | Active, Normal Gait |
| P846_1 WT | Non-injected | M | Active, Normal Gait |
| P846_5 WT | Non-injected | M | Active, Normal Gait |
| P846_6 WT | Non-injected | M | Active, Normal Gait |
| P846_12 WT | Non-injected | M | Active, Normal Gait |

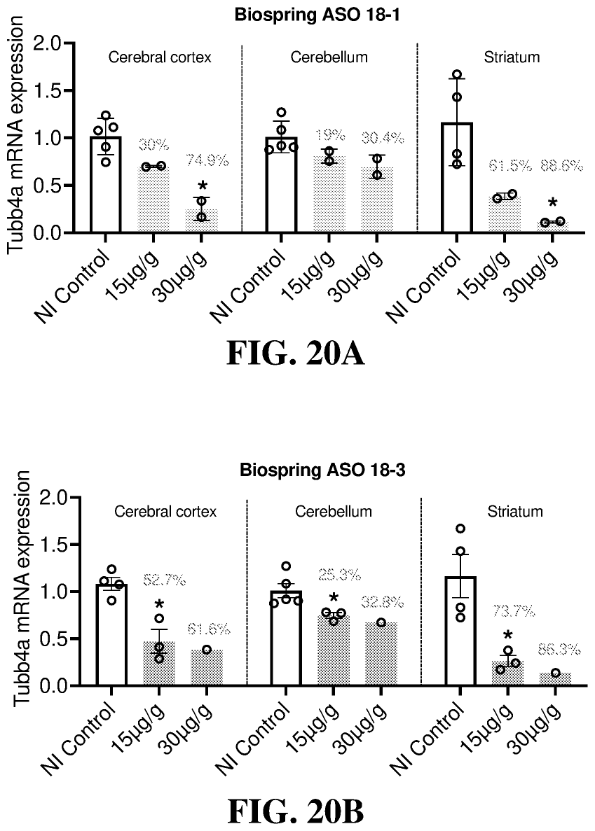

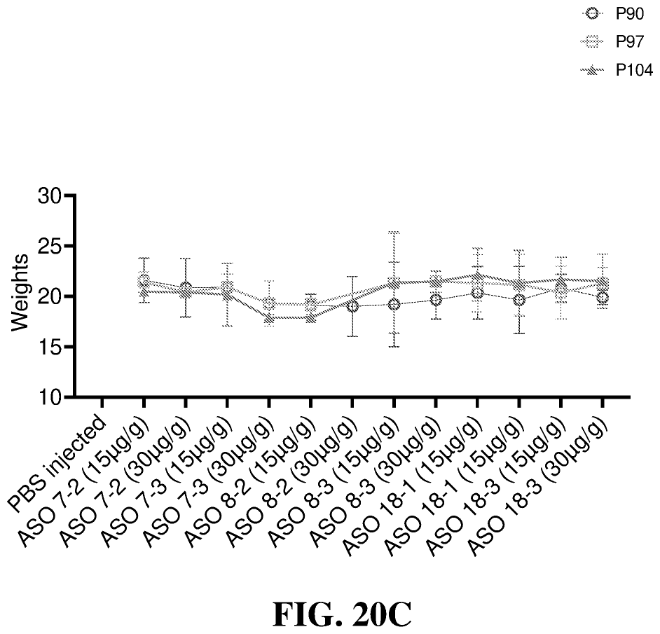

The neurological deficits cause by the ASOs were assessed in multiple ways. First, every week since injections, weights (FIG. 20C) and observations (listed in above Table 7) are taken to monitor the mice for weight loss and other adverse symptoms. Additionally, the general health and physical condition, spontaneous activity and additional reflexes and tone tests are performed.

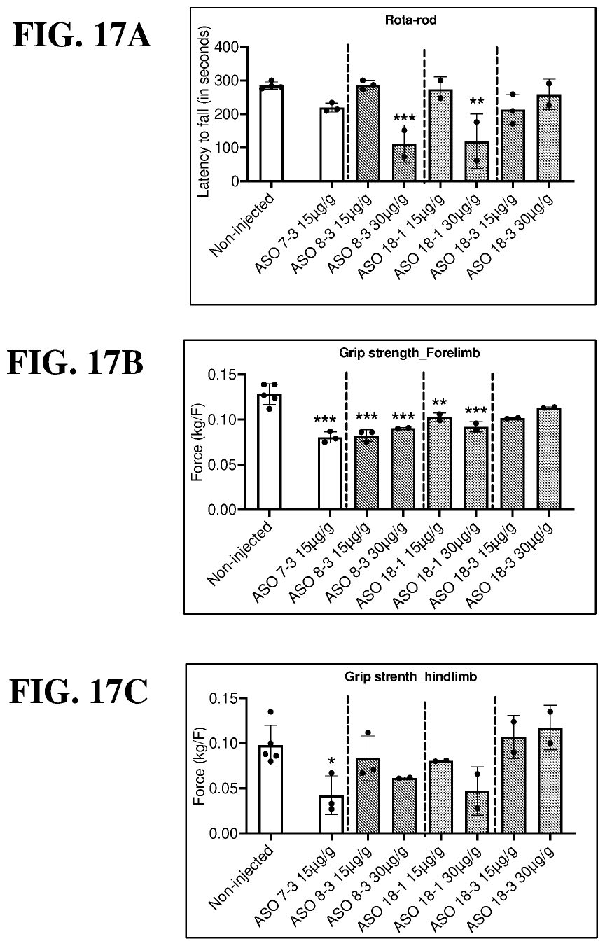

To assess the motor dysfunction after the ASO injection, a rotarod test is performed. In this test, the mouse is placed on a horizontally oriented, rotating cylinder suspended above the cage floor. The mouse attempts to stay on the rotating cylinder. The length of time the mouse stays on the cylinder is then recorded. This test is performed in three phases. On day 1, the acclimation phase, mice receive one trial for 100 seconds at a steady rate of 5 RPM. On day 2, the practice phase, mice received three trials for 300 seconds each on a gradual incline at 5-30 RPM. On Day 3, the test phase, mice receive three trials for 300 seconds each on a gradual incline at 5-30 RPM. The results of the test phase for each mouse, 30 days after injection, are shown in FIG. 17A.

To further assess the grip strength after the ASO injection, a grip strength test is performed. The grip strength of both the front limb and the hind limb is measured in kG/F units. Forelimb and hindlimb grip strength of ASO treated mice were tested using a grip strength meter (080312-3 Columbus Instruments, Columbus, OH, USA). For forelimb testing, mice were held at the proximal part of the tail and allowed to grasp a horizontal metal bar with both paws. The mice were then steadily pulled away and the pull force recorded once the mice unclasped the metal bar. For the hindlimb grip strength measurements, mice were allowed to grab the horizontal bar with their hindlimb paws while facing away from the meter and the tails were steadily pulled directly toward the meter until their grasps broke. Three trials were performed in each 2 min. FIGS. 17B and 17C shows the average results of the grip strength after three trials.

The in vivo toxicology of the ASOs was analyzed by collecting tissue and blood from each mouse. Tissues removed from the subjects include: blood, cortex, cerebellum, brain stem, midbrain, liver, and the rest of the brain. After removal, tissues were stored at −80° C. until the RNA and protein could be extracted. The summaries of the toxicology and Functional observation battery are provided in Table 8.

TABLE 8
Toxicity and FOB results

| Injection | Observations |
|---|---|
| ASO 7-2 | 3 Mice were injected |
| 15 μg/g | WT mice injected had to be euthanized because of severe ataxia, failure to |
| | walk and hunched ~d21-d30 post injection |
| ASO 7-2 | 3 Mice were injected |
| 30 μg/g | One died post one week after ASO injection |
| | One was euthanized post d23 after ASO injection because of severe |
| | ataxia, hunched, and not able to move (no striatum collected) |
| ASO 7-3 | 3 Mice were injected |
| 15 μg/g | Mild ataxia and d30 post injection |
| ASO 7-3 | 3 Mice were injected |
| 30 μg/g | Two were euthanized post d23 after ASO injection because of severe |
| | ataxia, hunched, and not able to move (no striatum collected) |
| ASO 8-2 | 3 Mice were injected |
| 15 μg/g | Two WT mice taken at P23 because of severe ataxia, were hunched and |
| | not able to move efficiently (no striatum collected). |
| | One striatum was collected on P30 |
| ASO 8-2 | 2 Mice were injected |
| 30 μg/g | Both deceased 2 weeks post ICV injection (no tissue collected) |
| ASO 8-3 | 3 Mice were injected |
| 15 μg/g | One WT mouse had ataxia, poor hindlimb grasp |
| | 2^{nd} mouse was hunched w/o ataxia |
| | 3^{rd} mouse was mild ataxic |
| | All tissues were collected post d30 injection |
| ASO 8-3 | 2 Mice were injected |
| 30 μg/g | Hunched, Mild ataxic and poor hindlimb tone and grasp |
| | Tissue collected for both |
| ASO 18-1 | 2 Mice were injected |
| 15 μg/g | Mild ataxia but all other parameters were good (active, alert, ect.) |
| ASO 18-1 | 2 Mice were injected |
| 30 μg/g | 1st mouse: Weak hindlimb tone, mild body tone, weak hindlimb grasp, |
| | head bobbing, wild twitching, absent tail elevation and dystonia. |
| | 2nd mouse: Poor hindlimb tone, ataxia, and dystonia. |
| ASO 18-3 | Three mice were injected |
| 15 μg/g | Mild hindlimb tone |
| ASO 18-3 | One mouse was injected |
| 30 μg/g | Mild hunched and poor hindlimb tone |

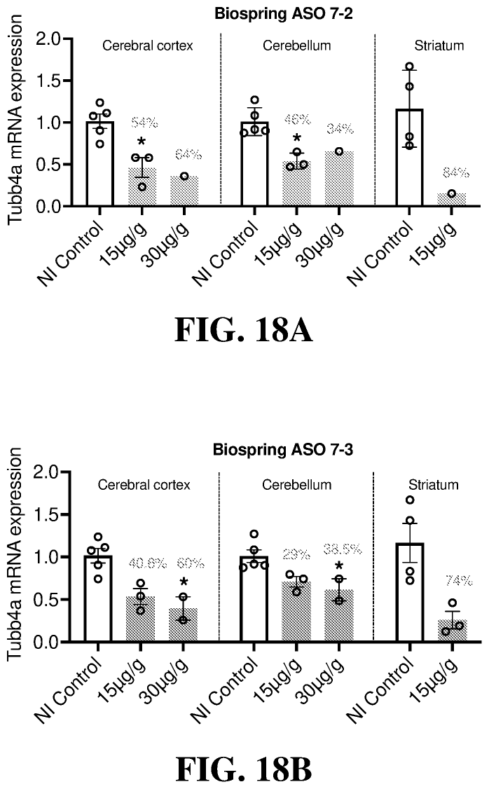

The downregulation of TUBB4A was further assessed using RNA extraction, cDNA creation and RT-qPCR. As indicated above, mice were given an ICV injected of ASO 7-2 at age P60 (adult). The subject's tissues were collected 23-30 days after injection. RNA was extracted and TUBB4A downregulation was determined by qRT-PCR. Results are shown in FIG. 18A. ASO 7-2 showed toxicity at both doses. For the 30 g/g dose, WT mice were euthanized humanely at earlier time points and therefore the values of downregulation for striatum are missing for 15 and 30 μg/g.

As indicated above, mice were given an ICV injected of ASO 7-3 at age P60 (adult). The subject's tissues were collected 23-30 days after injection. RNA was extracted and TUBB4A downregulation was determined by qRT-PCR. Results are shown in FIG. 18B. ASO 7-3 showed toxicity at both doses. As above, for the 30 μg/g dose, WT mice were euthanized humanely at earlier time points and therefore the values of downregulation for striatum are missing for 15 and 30 μg/g.

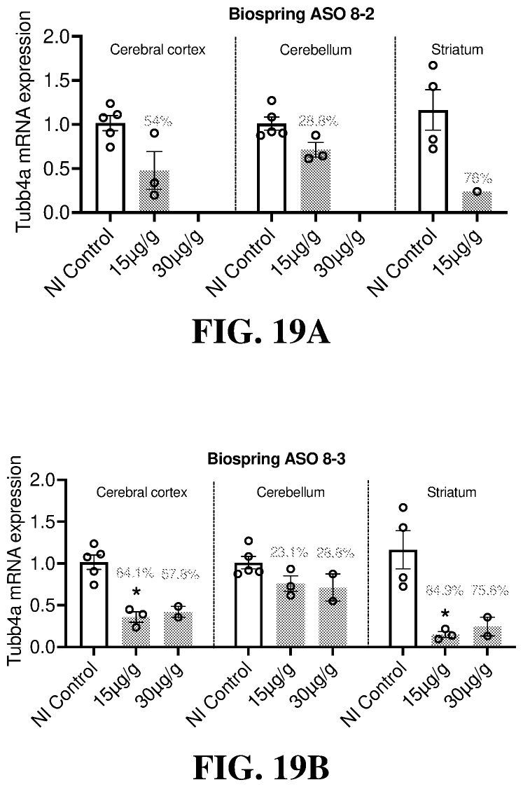

Mice were given an ICV injection of ASO 8-2 at age P60 (adult). The subject's tissues were collected 23-30 days after injection. RNA was extracted and TUBB4A downregulation was determined by qRT-PCR. Results are shown in FIG. 19A. ASO 8-2 showed lethality at 30 μg/g dose. Therefore, no tissues were collected at that dose.

As indicated above, mice were given an ICV injected of ASO 8-3 at age P60 (adult). The subject's tissues were collected 23-30 days after injection. RNA was extracted and TUBB4A downregulation was determined by qRT-PCR. Results are shown in FIG. 19B. ASO 8-3 did not show toxicity at either dose. All tissues were collected 30 days after injection.

As indicated above, mice were given an IC injected of ASO 18-1 at age P60 (adult). The subject's tissues were collected 23-30 days after injection. RNA was extracted and TUBB4A downregulation was determined by qRT-PCR. Results are shown in FIG. 20A. ASO 18-1 did not show toxicity at either dose. All tissues were collected 30 days after injection.

As indicated above, mice were given an ICV injected of ASO 18-3 at age P60 (adult). The subject's tissues were collected 23-30 days after injection. RNA was extracted and TUBB4A downregulation was determined by qRT-PCR. Results are shown in FIG. 20B. ASO 18-3 did not show toxicity at either dose. All tissues were collected 30 days after injection. In particular, ASO 18-3 showed both good TUBB4A downregulation and minimal toxicity in vivo.

Accordingly, ASOs 6-5, 7-5, 7-6, 8-5, and 18-5 were synthesized in order to produce good TUBB4A downregulation and minimal toxicity in vivo, similarly to ASO 18-3. Results of Tubb4a downregulation are provided in FIG. 15. The toxicity in vivo results are provided in Table 9. The toxicity of ASOs in reduced drastically.

TABLE 9
Status of Mice 31-28 days after injection and toxicity

| Name | Treatment | Sex | Observations |
|---|---|---|---|
| Mouse1 | PBS | M | Active, Normal Gait |
| Mouse2 | ASO6-5 (30 μg/g) | M | Active, Normal Gait |
| Mouse3 | ASO7-5 (30 μg/g) | M | Active, Normal Gait |
| Mouse4 | ASO 7-6 (15 μg/g) | M | Active, Normal Gait |
| Mouse5 | ASO 8-5 (15 μg/g) | F | Active, Normal Gait |
| Mouse6 | ASO 18-5 (15 μg/g) | F | Active, Normal Gait |
| Mouse7 | PBS | M | Active, Normal Gait |
| Mouse8 | ASO6-5 (30 μg/g) | M | Active, Normal Gait |
| Mouse9 | ASO7-5 (30 μg/g) | M | Active, Normal Gait |
| Mouse10 | ASO 7-6 (15 μg/g) | M | Active, Normal Gait |
| Mouse11 | ASO 8-5 (15 μg/g) | F | Active, Normal Gait |
| Mouse12 | ASO 18-5 (15 μg/g) | F | Active, Normal Gait |

### Example VI

### Methods for Downmodulating TUBB4A Activity for the Treatment of H-ABC Leukodystrophy

H-ABC and related TUBB4A associated leukodystrophy are currently untreatable. Based on the data provided in the Examples above, it is clear that downregulation of TUBB4A expression reduces H-ABC disease manifestations. Furthermore, it is clear that administration of the ASOs to patients is nontoxic. Notably, administration of antisense molecules has successfully been used for treatment of other neurodegenerative conditions, including Spinal Muscular Atrophy (See clinicaltrials.gov/ct2/show/NCT02122952). Following this paradigm, a patient having symptoms of H-ABC or a related TUBB4A-associated leukodystrophy can be treated via injection of an effective amount of an anti-TUBB4A antisense molecule using a method disclosed herein or in Evers et al., supra which down regulates TUBB4A expression, thereby alleviating symptoms of leukodystrophy.

### REFERENCES

Blumkin, L., et al., 2014. Expansion of the spectrum of TUBB4A-related disorders: a new phenotype associated with a novel mutation in the TUBB4A gene. Neurogenetics. 15, 107-13.
Ferreira, C., et al., 2014. Novel TUBB4A mutations and expansion of the neuroimaging phenotype of hypomyelination with atrophy of the basal ganglia and cerebellum (H-ABC). Am J Med Genet A. 164A, 1802-7.
Hersheson, J., et al., 2013. Mutations in the autoregulatory domain of beta-tubulin 4a cause hereditary dystonia. Ann Neurol. 73, 546-53.
Li, F. Y., et al., 2003. Mapping of taiep rat phenotype to rat Chromosome 9. Mamm Genome. 14, 703-5.
Pizzino, A., et al., 2014. TUBB4A de novo mutations cause isolated hypomyelination. Neurology. 83, 898-902.
Sase, S., et al., 2020. TUBB4A mutations result in both glial and neuronal degeneration in an H-ABC leukodystrophy mouse model. Elife. 9.
Simons, C., et al., 2013. A de novo mutation in the beta-tubulin gene TUBB4A results in the leukoencephalopathy hypomyelination with atrophy of the basal ganglia and cerebellum. Am J Hum Genet. 92, 767-73.

While certain features of the invention have been described herein, many modifications, substitutions, changes, and equivalents will now occur to those of ordinary skill in the art. It is, therefore, to be understood that the appended claims are intended to cover all such modifications and changes as fall within the true spirit of the invention.

1. A method of lowering TUBB4A levels in targeted cells, thereby improving symptoms of H-ABC leukodystrophy in a patient in need thereof, the method comprising administration of a therapeutically effective amount of a composition comprising a synthetic inhibitory nucleic acid molecule targeting TUBB4A, wherein lowering TUBB4A levels in said cell and reducing one or more of
i) delayed motor development;
ii) cognitive dysfunction;
iii) gait dysfunction;
iv) ataxia;
v) intention tremor;
vi) dysarthria;
vii) dysphonia; and
viii) aberrant hypomyelination;
in said patient, wherein the synthetic nucleic acid molecule is selected from an antisense oligonucleotide, an shRNA, an siRNA, and a guide strand suitable for CRISPR editing TUBB4A targeted nucleic acids.

2. A method of treating, delaying the onset of, ameliorating, and/or reducing a disease, disorder and/or condition, or a symptom thereof, associated with H-ABC leukodystrophy in a patient in need thereof, comprising administering to the patient a therapeutically effective amount of a synthetic nucleic acid targeting TUBB4A, wherein the disease, disorder and/or condition, or the symptom thereof, associated with H-ABC leukodystrophy is treated, inhibited, the onset delayed, ameliorated, and/or reduced in the patient, wherein the synthetic nucleic acid molecule is selected from an antisense oligonucleotide, an shRNA, an siRNA, and a guide strand suitable for CRISPR editing.

3. The method of claim 1, wherein said synthetic nucleic acid targeting TUBB4A is modified to increase stability and/or uptake in vivo.

4. The method of claim 1, wherein said synthetic nucleic acid targeting TUBB4A is a modified antisense oligonucleotide.

5. The method of claim 1, wherein said synthetic nucleic acid encoding said antisense oligonucleotide is cloned into a vector.

6. The method of claim 5, wherein said vector is selected from a plasmid vector, a lentiviral vector, a retroviral vector, an AAV vector, and an adenovirus associated vector.

7. The method of 6, wherein the disease, disorder and/or condition is associated with hypomyelination.

8. The method of claim 4, where said synthetic nucleic acid is an antisense oligonucleotide provided in Table 3 or Table 5.

9. The method of claim 8, wherein the nucleic acid is modified and has a nucleobase sequence that is at least 90%, at least 95%, at least 99%, or 100% complementary to a portion of a human TUBB4A nucleic acid.

10. The method of claim 9, wherein the modified oligonucleotide comprises at least one modified internucleoside linkage, at least one nucleoside of the modified oligonucleotide comprises a modified sugar or at least one nucleoside of the modified oligonucleotide comprises a modified nucleobase.

11. The method of claim 1, further comprising an additional active pharmaceutical agent useful for treatment of leukodystrophy.

12. A composition for reducing expression of TUBB4A, comprising a synthetic nucleic acid molecule targeting, and specifically hybridizing to, a TUBB4A encoding nucleic acid, selected from an antisense oligonucleotide, an shRNA, an siRNA, and a guide strand suitable for CRISPR editing in a biologically acceptable carrier.

13. The composition of claim 12, wherein said synthetic nucleic acid is modified to increase stability in bodily fluids and/or uptake in a cell of interest.

14. The composition of claim 12, wherein said synthetic nucleic acid is listed in Table 3 or Table 5.

15. The composition of 12, wherein said synthetic nucleic acid molecule is present in a vector.

16. The composition according to claim 12, formulated for ex vivo cellular administration, intracranial administration, parenteral administration, and intravenous administration.

17. The composition of 12 formulated for single intracerebroventricular (ICV) bolus injection.

18. A kit comprising for practicing the methods of claim 1.

19. The composition of claim 12, wherein said synthetic nucleic acid is ASO H14 or ASO H15.

20. The composition of claim 19, wherein said synthetic nucleic is present in vector selected from a plasmid vector, a lentiviral vector, a retroviral vector, an AAV vector, and an adenovirus associated vector, and is formulated for a route of administration selected from ex vivo cellular administration, intracranial administration, parenteral administration, intravenous administration, and single intracerebroventricular (ICV) bolus injection.


SEQUENCE LISTING (114 sequences)

SEQ ID NO: 1 - DNA, 16 bp, Artificial Sequence
  ASO H1
  modified_base (1)..(3): 2′-O-(2-Methoxyethyl)-oligori-bonucleotides
  misc_feature (1)..(16): phosphorothioate bonds
  modified_base (14)..(16): 2′-O-(2-Methoxyethyl)-oligori-bonucleotides
uaggtctcatccguau

SEQ ID NO: 2 - DNA, 16 bp, Artificial Sequence
  ASO H2
  modified_base (1)..(3): 2′-O-(2-Methoxyethyl)-oligori-bonucleotides
  misc_feature (1)..(16): phosphorothioate bonds
  modified_base (14)..(16): 2′-O-(2-Methoxyethyl)-oligori-bonucleotides
ugcacgctcagcaucu

SEQ ID NO: 3 - DNA, 15 bp, Artificial Sequence
  ASO H3
  misc_feature (1)..(15): phosphorothioate bonds
  modified_base (1)..(3): 2′-O-(2-Methoxyethyl)-oligori-bonucleotides
  modified_base (13)..(15): 2′-O-(2-Methoxyethyl)-oligori-bonucleotides
ucagaagcctcgagg

SEQ ID NO: 4 - DNA, 17 bp, Artificial Sequence
  ASO H4
  modified_base (1)..(4): 2′-O-(2-Methoxyethyl)-oligori-bonucleotides
  misc_feature (1)..(17): phosphorothioate bonds
  modified_base (14)..(17): 2′-O-(2-Methoxyethyl)-oligori-bonucleotides
gagctccaaaggtauug

SEQ ID NO: 5 - DNA, 17 bp, Artificial Sequence
  ASO H5
  misc_feature (1)..(17): phosphorothioate bonds
  modified_base (1)..(4): 2′-O-(2-Methoxyethyl)-oligori-bonucleotides
  modified_base (14)..(17): 2′-O-(2-Methoxyethyl)-oligori-bonucleotides
gcgagctccaaagguau

SEQ ID NO: 6 - DNA, 16 bp, Artificial Sequence
  ASO H6
  modified_base (1)..(3): 2′-O-(2-Methoxyethyl)-oligori-bonucleotides
  misc_feature (1)..(16): phosphorothioate bonds
  modified_base (14)..(16): 2′-O-(2-Methoxyethyl)-oligori-bonucleotides
agcgagctccaaaggu

SEQ ID NO: 7 - DNA, 16 bp, Artificial Sequence
  ASO H7
  misc_feature (1)..(16): phosphorothioate bonds
  modified_base (1)..(3): 2′-O-(2-Methoxyethyl)-oligori-bonucleotides
  modified_base (14)..(16): 2′-O-(2-Methoxyethyl)-oligori-bonucleotides
gguaaagcgagctcca

SEQ ID NO: 8 - DNA, 17 bp, Artificial Sequence
  ASO H8
  modified_base (1)..(4): 2′-O-(2-Methoxyethyl)-oligori-bonucleotides
  misc_feature (1)..(17): phosphorothioate bonds
  modified_base (14)..(17): 2′-O-(2-Methoxyethyl)-oligori-bonucleotides
gccagaggtaaagcgag

SEQ ID NO: 9 - DNA, 15 bp, Artificial Sequence
  ASO H9
  modified_base (1)..(3): 2′-O-(2-Methoxyethyl)-oligori-bonucleotides
  misc_feature (1)..(15): phosphorothioate bonds
  modified_base (13)..(15): 2′-O-(2-Methoxyethyl)-oligori-bonucleotides
ggutaaaggtgaggc

SEQ ID NO: 10 - DNA, 16 bp, Artificial Sequence
  ASO H10
  modified_base (1)..(3): 2′-O-(2-Methoxyethyl)-oligori-bonucleotides
  misc_feature (1)..(16): phosphorothioate bonds
  modified_base (14)..(16): 2′-O-(2-Methoxyethyl)-oligori-bonucleotides
ggacttgcaggtguag

SEQ ID NO: 11 - DNA, 15 bp, Artificial Sequence
  ASO H11
  misc_feature (1)..(15): phosphorothioate bonds
  modified_base (1)..(3): 2′-O-(2-Methoxyethyl)-oligori-bonucleotides
  modified_base (13)..(15): 2′-O-(2-Methoxyethyl)-oligori-bonucleotides
gaactgcagctcgga

SEQ ID NO: 12 - DNA, 17 bp, Artificial Sequence
  ASO H12
  modified_base (1)..(4): 2′-O-(2-Methoxyethyl)-oligori-bonucleotides
  misc_feature (1)..(17): phosphorothioate bonds
  modified_base (14)..(17): 2′-O-(2-Methoxyethyl)-oligori-bonucleotides
aagctaaggtcggcagg

SEQ ID NO: 13 - DNA, 18 bp, Artificial Sequence
  ASO H13
  misc_feature (1)..(18): phosphorothioate bonds
  modified_base (1)..(4): 2′-O-(2-Methoxyethyl)-oligori-bonucleotides
  modified_base (15)..(18): 2′-O-(2-Methoxyethyl)-oligori-bonucleotides
uaggtctcatccgtauuc

SEQ ID NO: 14 - DNA, 17 bp, Artificial Sequence
  ASO H14
  modified_base (1)..(4): 2′-O-(2-Methoxyethyl)-oligori-bonucleotides
  misc_feature (1)..(17): phosphorothioate bonds
  modified_base (14)..(17): 2′-O-(2-Methoxyethyl)-oligori-bonucleotides
guaggtctcatccguau

SEQ ID NO: 15 - DNA, 15 bp, Artificial Sequence
  ASO H15
  misc_feature (1)..(15): phosphorothioate bonds
  modified_base (1)..(3): 2′-O-(2-Methoxyethyl)-oligori-bonucleotides
  modified_base (13)..(15): 2′-O-(2-Methoxyethyl)-oligori-bonucleotides
ggucagaagcctcga

SEQ ID NO: 16 - DNA, 18 bp, Artificial Sequence
  ASO H16
  modified_base (1)..(4): 2′-O-(2-Methoxyethyl)-oligori-bonucleotides
  misc_feature (1)..(18): phosphorothioate bonds
  modified_base (15)..(18): 2′-O-(2-Methoxyethyl)-oligori-bonucleotides
gcgagctccaaagguauu

SEQ ID NO: 17 - DNA, 18 bp, Artificial Sequence
  ASO H17
  misc_feature (1)..(18): phosphorothioate bonds
  modified_base (1)..(4): 2′-O-(2-Methoxyethyl)-oligori-bonucleotides
  modified_base (15)..(18): 2′-O-(2-Methoxyethyl)-oligori-bonucleotides
agcgagctccaaagguau

SEQ ID NO: 18 - DNA, 17 bp, Artificial Sequence
  ASO H18
  modified_base (1)..(4): 2′-O-(2-Methoxyethyl)-oligori-bonucleotides
  misc_feature (1)..(17): phosphorothioate bonds
  modified_base (14)..(17): 2′-O-(2-Methoxyethyl)-oligori-bonucleotides
agcgagctccaaaggua

SEQ ID NO: 19 - DNA, 18 bp, Artificial Sequence
  ASO H19
  misc_feature (1)..(18): phosphorothioate bonds
  modified_base (1)..(4): 2′-O-(2-Methoxyethyl)-oligori-bonucleotides
  modified_base (15)..(18): 2′-O-(2-Methoxyethyl)-oligori-bonucleotides
aaagcgagctccaaaggu

SEQ ID NO: 20 - DNA, 17 bp, Artificial Sequence
  ASO H20
  modified_base (1)..(4): 2′-O-(2-Methoxyethyl)-oligori-bonucleotides
  misc_feature (1)..(17): phosphorothioate bonds
  modified_base (14)..(17): 2′-O-(2-Methoxyethyl)-oligori-bonucleotides
aaagcgagctccaaagg

SEQ ID NO: 21 - DNA, 17 bp, Artificial Sequence
  ASO H21
  misc_feature (1)..(17): phosphorothioate bonds
  modified_base (1)..(4): 2′-O-(2-Methoxyethyl)-oligori-bonucleotides
  modified_base (14)..(17): 2′-O-(2-Methoxyethyl)-oligori-bonucleotides
ggacttgcaggtguaga

SEQ ID NO: 22 - DNA, 16 bp, Artificial Sequence
  ASO H2A
  modified_base (1)..(4): 2′-O-(2-Methoxyethyl)-oligori-bonucleotides
  misc_feature (1)..(2): phosphorothioate bonds
  misc_feature (5)..(13): phosphorothioate bonds
  modified_base (5)..(5): 5′methyl
  modified_base (7)..(7): 5′methyl
  modified_base (9)..(9): 5′methyl
  modified_base (12)..(12): 5′methyl
  modified_base (13)..(16): 2′-O-(2-Methoxyethyl)-oligori-bonucleotides
ugcacgctcagcaucu

SEQ ID NO: 23 - DNA, 16 bp, Artificial Sequence
  ASO H2B
  misc_feature (1)..(2): phosphorothioate bonds
  modified_base (1)..(4): 2′-O-(2-Methoxyethyl)-oligori-bonucleotides
  modified_base (5)..(5): 5′methyl
  misc_feature (5)..(13): phosphorothioate bonds
  modified_base (7)..(7): 5′methyl
  modified_base (9)..(9): 5′methyl
  modified_base (12)..(12): 5′methyl
  modified_base (14)..(16): 2′-O-(2-Methoxyethyl)-oligori-bonucleotides
  misc_feature (15)..(16): phosphorothioate bonds
ugcacgctcagcaucu

SEQ ID NO: 24 - DNA, 16 bp, Artificial Sequence
  ASO H10A
  modified_base (1)..(4): 2′-O-(2-Methoxyethyl)-oligori-bonucleotides
  misc_feature (1)..(2): phosphorothioate bonds
  misc_feature (5)..(13): phosphorothioate bonds
  modified_base (8)..(8): 5′methyl
  modified_base (13)..(16): 2′-O-(2-Methoxyethyl)-oligori-bonucleotides
ggacttgcaggtguag

SEQ ID NO: 25 - DNA, 16 bp, Artificial Sequence
  ASO H10B
  misc_feature (1)..(2): phosphorothioate bonds
  modified_base (1)..(4): 2′-O-(2-Methoxyethyl)-oligori-bonucleotides
  misc_feature (5)..(13): phosphorothioate bonds
  modified_base (8)..(8): 5′methyl
  modified_base (14)..(16): 2′-O-(2-Methoxyethyl)-oligori-bonucleotides
  misc_feature (15)..(16): phosphorothioate bonds
ggacttgcaggtguag

SEQ ID NO: 26 - DNA, 15 bp, Artificial Sequence
  ASO H11A
  modified_base (1)..(3): 2′-O-(2-Methoxyethyl)-oligori-bonucleotides
  misc_feature (1)..(2): phosphorothioate bonds
  modified_base (4)..(4): 5′methyl
  misc_feature (5)..(12): phosphorothioate bonds
  modified_base (7)..(7): 5′methyl
  modified_base (10)..(10): 5′methyl
  modified_base (12)..(12): 5′methyl
  modified_base (13)..(15): 2′-O-(2-Methoxyethyl)-oligori-bonucleotides
gaactgcagctcgga

SEQ ID NO: 27 - DNA, 15 bp, Artificial Sequence
  ASO H11B
  misc_feature (1)..(2): phosphorothioate bonds
  modified_base (1)..(3): 2′-O-(2-Methoxyethyl)-oligori-bonucleotides
  modified_base (4)..(4): 5′methyl
  misc_feature (5)..(12): phosphorothioate bonds
  modified_base (7)..(7): 5′methyl
  modified_base (10)..(10): 5′methyl
  modified_base (12)..(12): 5′methyl
  modified_base (13)..(15): 2′-O-(2-Methoxyethyl)-oligori-bonucleotides
  misc_feature (14)..(15): phosphorothioate bonds
gaactgcagctcgga

SEQ ID NO: 28 - DNA, 15 bp, Artificial Sequence
  ASO H15A
  modified_base (1)..(3): 2′-O-(2-Methoxyethyl)-oligori-bonucleotides
  misc_feature (1)..(2): phosphorothioate bonds
  modified_base (4)..(4): 5′methyl
  misc_feature (5)..(12): phosphorothioate bonds
  modified_base (10)..(11): 5′methyl
  modified_base (13)..(15): 2′-O-(2-Methoxyethyl)-oligori-bonucleotides
ggucagaagcctcga

SEQ ID NO: 29 - DNA, 15 bp, Artificial Sequence
  ASO H15B
  misc_feature (1)..(2): phosphorothioate bonds
  modified_base (1)..(3): 2′-O-(2-Methoxyethyl)-oligori-bonucleotides
  modified_base (4)..(4): 5′methyl
  misc_feature (5)..(12): phosphorothioate bonds
  modified_base (10)..(11): 5′methyl
  modified_base (13)..(15): 2′-O-(2-Methoxyethyl)-oligori-bonucleotides
  misc_feature (14)..(15): phosphorothioate bonds
ggucagaagcctcga

SEQ ID NO: 30 - DNA, 17 bp, Artificial Sequence
  ASO 1
  modified_base (1)..(4): 2′-O-(2-Methoxyethyl)-oligori-bonucleotides
  misc_feature (1)..(17): phosphorothioate bonds
  modified_base (14)..(17): 2′-O-(2-Methoxyethyl)-oligori-bonucleotides
gcuugcaggtgcacgau

SEQ ID NO: 31 - DNA, 17 bp, Artificial Sequence
  ASO 2
  misc_feature (1)..(17): phosphorothioate bonds
  modified_base (1)..(4): 2′-O-(2-Methoxyethyl)-oligori-bonucleotides
  modified_base (14)..(17): 2′-O-(2-Methoxyethyl)-oligori-bonucleotides
ugucgatgcagtagguc

SEQ ID NO: 32 - DNA, 16 bp, Artificial Sequence
  ASO 3
  modified_base (1)..(3): 2′-O-(2-Methoxyethyl)-oligori-bonucleotides
  misc_feature (1)..(16): phosphorothioate bonds
  modified_base (14)..(16): 2′-O-(2-Methoxyethyl)-oligori-bonucleotides
ugucgatgcagtaggu

SEQ ID NO: 33 - DNA, 17 bp, Artificial Sequence
  ASO 4
  misc_feature (1)..(17): phosphorothioate bonds
  modified_base (1)..(4): 2′-O-(2-Methoxyethyl)-oligori-bonucleotides
  modified_base (14)..(17): 2′-O-(2-Methoxyethyl)-oligori-bonucleotides
ccucgttgtcgatgcag

SEQ ID NO: 34 - DNA, 17 bp, Artificial Sequence
  ASO 5
  modified_base (1)..(4): 2′-O-(2-Methoxyethyl)-oligori-bonucleotides
  misc_feature (1)..(17): * phosphorothioate bonds
  modified_base (14)..(17): 2′-O-(2-Methoxyethyl)-oligori-bonucleotides
uugaggtccccgtaggu

SEQ ID NO: 35 - DNA, 17 bp, Artificial Sequence
  ASO 6
  misc_feature (1)..(17): phosphorothioate bonds
  modified_base (1)..(4): 2′-O-(2-Methoxyethyl)-oligori-bonucleotides
  modified_base (14)..(17): 2′-O-(2-Methoxyethyl)-oligori-bonucleotides
gcucgtctacctccuuc

SEQ ID NO: 36 - DNA, 16 bp, Artificial Sequence
  ASO 7
  modified_base (1)..(3): 2′-O-(2-Methoxyethyl)-oligori-bonucleotides
  misc_feature (1)..(16): phosphorothioate bonds
  modified_base (14)..(16): 2′-O-(2-Methoxyethyl)-oligori-bonucleotides
gcucgtctacctccuu

SEQ ID NO: 37 - DNA, 16 bp, Artificial Sequence
  ASO 8
  misc_feature (1)..(16): phosphorothioate bonds
  modified_base (1)..(3): 2′-O-(2-Methoxyethyl)-oligori-bonucleotides
  modified_base (14)..(16): 2′-O-(2-Methoxyethyl)-oligori-bonucleotides
ugctcgtctacctccu

SEQ ID NO: 38 - DNA, 17 bp, Artificial Sequence
  ASO 9
  modified_base (1)..(4): 2′-O-(2-Methoxyethyl)-oligori-bonucleotides
  misc_feature (1)..(17): phosphorothioate bonds
  modified_base (14)..(17): 2′-O-(2-Methoxyethyl)-oligori-bonucleotides
ucucgtccatgccuucg

SEQ ID NO: 39 - DNA, 17 bp, Artificial Sequence
  ASO 10
  misc_feature (1)..(17): phosphorothioate bonds
  modified_base (1)..(4): 2′-O-(2-Methoxyethyl)-oligori-bonucleotides
  modified_base (14)..(17): 2′-O-(2-Methoxyethyl)-oligori-bonucleotides
ccauctcgtccatgccu

SEQ ID NO: 40 - DNA, 17 bp, Artificial Sequence
  ASO 11
  modified_base (1)..(4): 2′-O-(2-Methoxyethyl)-oligori-bonucleotides
  misc_feature (1)..(17): phosphorothioate bonds
  modified_base (14)..(17): 2′-O-(2-Methoxyethyl)-oligori-bonucleotides
ucuucgaactcgcccuc

SEQ ID NO: 41 - DNA, 16 bp, Artificial Sequence
  ASO 12
  misc_feature (1)..(16): phosphorothioate bonds
  modified_base (1)..(3): 2′-O-(2-Methoxyethyl)-oligori-bonucleotides
  modified_base (14)..(16): 2′-O-(2-Methoxyethyl)-oligori-bonucleotides
ccucctcttcgaacuc

SEQ ID NO: 42 - DNA, 15 bp, Artificial Sequence
  ASO 13
  modified_base (1)..(3): 2′-O-(2-Methoxyethyl)-oligori-bonucleotides
  modified_base (13)..(15): 2′-O-(2-Methoxyethyl)-oligori-bonucleotides
  misc_feature (13)..(15): phosphorothioate bonds
ggucagaggtaaggc

SEQ ID NO: 43 - DNA, 17 bp, Artificial Sequence
  ASO 14
  misc_feature (1)..(17): phosphorothioate bonds
  modified_base (1)..(4): 2′-O-(2-Methoxyethyl)-oligori-bonucleotides
  modified_base (14)..(17): 2′-O-(2-Methoxyethyl)-oligori-bonucleotides
ugcacgatttcccgcau

SEQ ID NO: 44 - DNA, 17 bp, Artificial Sequence
  ASO 15
  modified_base (1)..(4): 2′-O-(2-Methoxyethyl)-oligori-bonucleotides
  misc_feature (1)..(17): phosphorothioate bonds
  modified_base (14)..(17): 2′-O-(2-Methoxyethyl)-oligori-bonucleotides
guugtcgatgcaguagg

SEQ ID NO: 45 - DNA, 19 bp, Artificial Sequence
  ASO 16
  misc_feature (1)..(19): phosphorothioate bonds
  modified_base (1)..(5): 2′-O-(2-Methoxyethyl)-oligori-bonucleotides
  modified_base (16)..(19): 2′-O-(2-Methoxyethyl)-oligori-bonucleotides
ccucgttgtcgatgcagua

SEQ ID NO: 46 - DNA, 17 bp, Artificial Sequence
  ASO 17
  modified_base (1)..(4): 2′-O-(2-Methoxyethyl)-oligori-bonucleotides
  misc_feature (1)..(17): phosphorothioate bonds
  modified_base (15)..(17): 2′-O-(2-Methoxyethyl)-oligori-bonucleotides
cucgttgtcgatgcagu

SEQ ID NO: 47 - DNA, 15 bp, Artificial Sequence
  ASO 18
  misc_feature (1)..(15): phosphorothioate bonds
  modified_base (1)..(3): 2′-O-(2-Methoxyethyl)-oligori-bonucleotides
  modified_base (13)..(15): 2′-O-(2-Methoxyethyl)-oligori-bonucleotides
ugctcgtctaccucc

SEQ ID NO: 48 - DNA, 18 bp, Artificial Sequence
  ASO 19
  modified_base (1)..(4): 2′-O-(2-Methoxyethyl)-oligori-bonucleotides
  modified_base (15)..(18): 2′-O-(2-Methoxyethyl)-oligori-bonucleotides
  misc_feature (15)..(18): phosphorothioate bonds
gugctgttgccgatgaag

SEQ ID NO: 49 - DNA, 16 bp, Artificial Sequence
  ASO 20
  misc_feature (1)..(16): phosphorothioate bonds
  modified_base (1)..(3): 2′-O-(2-Methoxyethyl)-oligori-bonucleotides
  modified_base (14)..(16): 2′-O-(2-Methoxyethyl)-oligori-bonucleotides
gugctgttgccgauga

SEQ ID NO: 50 - DNA, 19 bp, Artificial Sequence
  ASO 21
  modified_base (1)..(5): 2′-O-(2-Methoxyethyl)-oligori-bonucleotides
  misc_feature (1)..(19): phosphorothioate bonds
  modified_base (16)..(19): 2′-O-(2-Methoxyethyl)-oligori-bonucleotides
aucucgtccatgcctucgc

SEQ ID NO: 51 - DNA, 18 bp, Artificial Sequence
  ASO 22
  misc_feature (1)..(18): phosphorothioate bonds
  modified_base (1)..(4): 2′-O-(2-Methoxyethyl)-oligori-bonucleotides
  modified_base (15)..(18): 2′-O-(2-Methoxyethyl)-oligori-bonucleotides
ccauctcgtccatgccuu

SEQ ID NO: 52 - DNA, 17 bp, Artificial Sequence
  ASO 23
  modified_base (1)..(4): 2′-O-(2-Methoxyethyl)-oligori-bonucleotides
  misc_feature (1)..(17): phosphorothioate bonds
  modified_base (14)..(17): 2′-O-(2-Methoxyethyl)-oligori-bonucleotides
uccatctcgtccaugcc

SEQ ID NO: 53 - DNA, 16 bp, Artificial Sequence
  ASO 24
  misc_feature (1)..(16): phosphorothioate bonds
  modified_base (1)..(3): 2′-O-(2-Methoxyethyl)-oligori-bonucleotides
  modified_base (14)..(16): 2′-O-(2-Methoxyethyl)-oligori-bonucleotides
uucgaactcgcccucu

SEQ ID NO: 54 - DNA, 17 bp, Artificial Sequence
  ASO 25
  modified_base (1)..(4): 2′-O-(2-Methoxyethyl)-oligori-bonucleotides
  modified_base (14)..(17): 2′-O-(2-Methoxyethyl)-oligori-bonucleotides
  misc_feature (14)..(17): phosphorothioate bonds
cucutcgaactcgcccu

SEQ ID NO: 55 - DNA, 17 bp, Artificial Sequence
  ASO 4A
  misc_feature (1)..(2): phosphorothioate bonds
  modified_base (1)..(4): 2′-O-(2-Methoxyethyl)-oligori-bonucleotides
  misc_feature (5)..(14): phosphorothioate bonds
  modified_base (10)..(10): 5′methyl
  modified_base (14)..(17): 2′-O-(2-Methoxyethyl)-oligori-bonucleotides
ccucgttgtcgatgcag

SEQ ID NO: 56 - DNA, 17 bp, Artificial Sequence
  ASO 4B
  modified_base (1)..(4): 2′-O-(2-Methoxyethyl)-oligori-bonucleotides
  misc_feature (1)..(2): phosphorothioate bonds
  misc_feature (5)..(14): phosphorothioate bonds
  modified_base (10)..(10): 5′methyl
  modified_base (14)..(17): 2′-O-(2-Methoxyethyl)-oligori-bonucleotides
  misc_feature (16)..(17): phosphorothioate bonds
ccucgttgtcgatgcag

SEQ ID NO: 57 - DNA, 17 bp, Artificial Sequence
  ASO 6A
  misc_feature (1)..(2): phosphorothioate bonds
  modified_base (1)..(4): 2′-O-(2-Methoxyethyl)-oligori-bonucleotides
  misc_feature (5)..(14): phosphorothioate bonds
  modified_base (7)..(7): 5′methyl
  modified_base (10)..(11): 5′methyl
  modified_base (14)..(17): 2′-O-(2-Methoxyethyl)-oligori-bonucleotides
gcucgtctacctccuuc

SEQ ID NO: 58 - DNA, 17 bp, Artificial Sequence
  ASO 6B
  modified_base (1)..(4): 2′-O-(2-Methoxyethyl)-oligori-bonucleotides
  misc_feature (1)..(2): phosphorothioate bonds
  misc_feature (5)..(14): phosphorothioate bonds
  modified_base (7)..(7): 5′methyl
  modified_base (10)..(11): 5′methyl
  modified_base (14)..(17): 2′-O-(2-Methoxyethyl)-oligori-bonucleotides
  misc_feature (16)..(17): phosphorothioate bonds
gcucgtctacctccuuc

SEQ ID NO: 59 - DNA, 16 bp, Artificial Sequence
  ASO 7A
  misc_feature (1)..(2): phosphorothioate bonds
  modified_base (1)..(4): 2′-O-(2-Methoxyethyl)-oligori-bonucleotides
  misc_feature (5)..(13): phosphorothioate bonds
  modified_base (7)..(7): 5′methyl
  modified_base (10)..(11): 5′methyl
  modified_base (13)..(13): 5′methyl
  modified_base (14)..(16): 2′-O-(2-Methoxyethyl)-oligori-bonucleotides
gcucgtctacctccuu

SEQ ID NO: 60 - DNA, 16 bp, Artificial Sequence
  ASO 7B
  modified_base (1)..(4): 2′-O-(2-Methoxyethyl)-oligori-bonucleotides
  misc_feature (1)..(2): phosphorothioate bonds
  misc_feature (5)..(13): phosphorothioate bonds
  modified_base (7)..(7): 5′methyl
  modified_base (10)..(11): 5′methyl
  modified_base (13)..(13): 5′methyl
  modified_base (14)..(16): 2′-O-(2-Methoxyethyl)-oligori-bonucleotides
  misc_feature (15)..(16): phosphorothioate bonds
gcucgtctacctccuu

SEQ ID NO: 61 - DNA, 16 bp, Artificial Sequence
  NTC Control
  misc_feature (1)..(2): phosphorothioate bonds
  modified_base (1)..(4): 2′-O-(2-Methoxyethyl)-oligori-bonucleotides
  misc_feature (5)..(13): phosphorothioate bonds
  modified_base (5)..(5): 5′methyl
  modified_base (13)..(16): 2′-O-(2-Methoxyethyl)-oligori-bonucleotides
  misc_feature (15)..(16): phosphorothioate bonds
uacgcgacuuaugcgg

SEQ ID NO: 62 - DNA, 16 bp, Artificial Sequence
  ASO H2-1
  misc_feature (1)..(3): 2′-O-(2-Methoxyethyl)-oligori-bonucleotides
  misc_feature (1)..(15): phosphorothioate bonds
  misc_feature (3)..(3): 5′methyl
  misc_feature (5)..(5): 5′methyl
  misc_feature (7)..(7): 5′methyl
  misc_feature (9)..(9): 5′methyl
  misc_feature (12)..(12): 5′methyl
  misc_feature (14)..(16): 2′-O-(2-Methoxyethyl)-oligori-bonucleotides
  misc_feature (15)..(15): 5′methyl
ugcacgctcagcaucu

SEQ ID NO: 63 - DNA, 16 bp, Artificial Sequence
  ASO H2-2
  misc_feature (1)..(1): 2′-O-(2-Methoxyethyl)-oligori-bonucleotides
  misc_feature (1)..(15): phosphorothioate bonds
  misc_feature (2)..(2): locked nucleic acid
  misc_feature (3)..(3): 2′-O-(2-Methoxyethyl)-oligori-bonucleotides
  misc_feature (3)..(3): 5′methyl
  misc_feature (5)..(5): 5′methyl
  misc_feature (7)..(7): 5′methyl
  misc_feature (9)..(9): 5′methyl
  misc_feature (12)..(12): 5′methyl
  misc_feature (14)..(14): 2′-O-(2-Methoxyethyl)-oligori-bonucleotides
  misc_feature (15)..(15): 5′methyl
  misc_feature (15)..(15): locked nucleic acid
  misc_feature (16)..(16): 2′-O-(2-Methoxyethyl)-oligori-bonucleotides
ugcacgctcagcaucu

SEQ ID NO: 64 - DNA, 16 bp, Artificial Sequence
  ASO H2-3
  misc_feature (1)..(3): locked nucleic acid
  misc_feature (1)..(16): phosphorothioate bonds
  misc_feature (3)..(3): 5′methyl
  misc_feature (5)..(5): 5′methyl
  misc_feature (7)..(7): 5′methyl
  misc_feature (9)..(9): 5′methyl
  misc_feature (12)..(12): 5′methyl
  misc_feature (15)..(17): locked nucleic acid
  misc_feature (16)..(16): 5′methyl
ugcacgctcagcaucu

SEQ ID NO: 65 - DNA, 18 bp, Artificial Sequence
  ASO H2-4
  misc_feature (1)..(4): 2′-O-(2-Methoxyethyl)-oligori-bonucleotides
  misc_feature (1)..(17): phosphorothioate bonds
  misc_feature (1)..(1): 5′methyl
  misc_feature (4)..(4): 5′methyl
  misc_feature (6)..(6): 5′methyl
  misc_feature (8)..(8): 5′methyl
  misc_feature (10)..(10): 5′methyl
  misc_feature (13)..(13): 5′methyl
  misc_feature (15)..(18): 2′-O-(2-Methoxyethyl)-oligori-bonucleotides
  misc_feature (16)..(16): 5′methyl
cugcacgctcagcaucug

SEQ ID NO: 66 - DNA, 16 bp, Artificial Sequence
  ASO H10-1
  misc_feature (1)..(3): 2′-O-(2-Methoxyethyl)-oligori-bonucleotides
  misc_feature (1)..(15): phosphorothioate bonds
  misc_feature (4)..(4): 5′methyl
  misc_feature (8)..(8): 5′methyl
  misc_feature (14)..(16): 2′-O-(2-Methoxyethyl)-oligori-bonucleotides
ggacttgcaggtguag

SEQ ID NO: 67 - DNA, 16 bp, Artificial Sequence
  ASO H10-2
  misc_feature (1)..(3): locked nucleic acid
  misc_feature (1)..(1): phosphorothioate bonds
  misc_feature (3)..(13): phosphorothioate bonds
  misc_feature (4)..(4): 5′methyl
  misc_feature (8)..(8): 5′methyl
  misc_feature (14)..(16): locked nucleic acid
  misc_feature (15)..(16): phosphorothioate bonds
ggacttgcaggtguag

SEQ ID NO: 68 - DNA, 18 bp, Artificial Sequence
  ASO H10-3
  misc_feature (1)..(4): 2′-O-(2-Methoxyethyl)-oligori-bonucleotides
  misc_feature (1)..(2): 2′-O-(2-Methoxyethyl)-oligori-bonucleotides
  misc_feature (4)..(15): 2′-O-(2-Methoxyethyl)-oligori-bonucleotides
  misc_feature (5)..(5): 5′methyl
  misc_feature (9)..(9): 5′methyl
  misc_feature (15)..(18): 2′-O-(2-Methoxyethyl)-oligori-bonucleotides
  misc_feature (17)..(18): 2′-O-(2-Methoxyethyl)-oligori-bonucleotides
aggacttgcaggtguaga

SEQ ID NO: 69 - DNA, 16 bp, Artificial Sequence
  ASO H10-4
  misc_feature (1)..(3): locked nucleic acid
  misc_feature (3)..(14): phosphorothioate bonds
  misc_feature (4)..(4): 5′methyl
  misc_feature (8)..(8): 5′methyl
  misc_feature (14)..(16): locked nucleic acid
ggacttgcaggtguag

SEQ ID NO: 70 - DNA, 15 bp, Artificial Sequence
  ASO H11-1
  misc_feature (1)..(3): 2′-O-(2-Methoxyethyl)-oligori-bonucleotides
  misc_feature (1)..(13): phosphorothioate bonds
  misc_feature (4)..(4): 5′methyl
  misc_feature (7)..(7): 5′methyl
  misc_feature (10)..(10): 5′methyl
  misc_feature (12)..(12): 5′methyl
  misc_feature (13)..(15): 2′-O-(2-Methoxyethyl)-oligori-bonucleotides
gaactgcagctcgga

SEQ ID NO: 71 - DNA, 15 bp, Artificial Sequence
  ASO H11-2
  misc_feature (1)..(14): phosphorothioate bonds
  misc_feature (1)..(1): locked nucleic acid
  misc_feature (2)..(3): 2′-O-(2-Methoxyethyl)-oligori-bonucleotides
  misc_feature (4)..(4): 5′methyl
  misc_feature (7)..(7): 5′methyl
  misc_feature (10)..(10): 5′methyl
  misc_feature (12)..(12): 5′methyl
  misc_feature (13)..(14): 2′-O-(2-Methoxyethyl)-oligori-bonucleotides
  misc_feature (15)..(15): locked nucleic acid
gaactgcagctcgga

SEQ ID NO: 72 - DNA, 17 bp, Artificial Sequence
  ASO H11-3
  misc_feature (1)..(16): phosphorothioate bonds
  misc_feature (1)..(1): locked nucleic acid
  misc_feature (2)..(4): 2′-O-(2-Methoxyethyl)-oligori-bonucleotides
  misc_feature (5)..(5): 5′methyl
  misc_feature (8)..(8): 5′methyl
  misc_feature (11)..(11): 5′methyl
  misc_feature (13)..(13): 5′methyl
  misc_feature (14)..(16): 2′-O-(2-Methoxyethyl)-oligori-bonucleotides
  misc_feature (17)..(17): locked nucleic acid
ugaactgcagctcggag

SEQ ID NO: 73 - DNA, 17 bp, Artificial Sequence
  ASO H14-1
  misc_feature (1)..(4): 2′-O-(2-Methoxyethyl)-oligori-bonucleotides
  misc_feature (4)..(14): phosphorothioate bonds
  misc_feature (7)..(7): 5′methyl
  misc_feature (9)..(9): 5′methyl
  misc_feature (12)..(13): 5′methyl
  misc_feature (14)..(17): 2′-O-(2-Methoxyethyl)-oligori-bonucleotides
guaggtctcatccguau

SEQ ID NO: 74 - DNA, 15 bp, Artificial Sequence
  ASO H15-1
  misc_feature (1)..(3): 2′-O-(2-Methoxyethyl)-oligori-bonucleotides
  misc_feature (1)..(14): phosphorothioate bonds
  misc_feature (4)..(4): 5′methyl
  misc_feature (10)..(11): 5′methyl
  misc_feature (13)..(13): 5′methyl
  misc_feature (13)..(15): 2′-O-(2-Methoxyethyl)-oligori-bonucleotides
ggucagaagcctcga

SEQ ID NO: 75 - DNA, 15 bp, Artificial Sequence
  ASO H15-2
  misc_feature (1)..(14): phosphorothioate bonds
  misc_feature (1)..(2): locked nucleic acid
  misc_feature (3)..(3): 2′-O-(2-Methoxyethyl)-oligori-bonucleotides
  misc_feature (4)..(4): 5′methyl
  misc_feature (10)..(11): 5′methyl
  misc_feature (13)..(13): 5′methyl
  misc_feature (13)..(13): 2′-O-(2-Methoxyethyl)-oligori-bonucleotides
  misc_feature (14)..(15): locked nucleic acid
ggucagaagcctcga

SEQ ID NO: 76 - DNA, 19 bp, Artificial Sequence
  ASO H15-3
  misc_feature (1)..(5): 2′-O-(2-Methoxyethyl)-oligori-bonucleotides
  misc_feature (1)..(18): phosphorothioate bonds
  misc_feature (6)..(6): 5′methyl
  misc_feature (12)..(13): 5′methyl
  misc_feature (15)..(15): 5′methyl
  misc_feature (15)..(19): 2′-O-(2-Methoxyethyl)-oligori-bonucleotides
aaggucagaagcctcgagg

SEQ ID NO: 77 - DNA, 15 bp, Artificial Sequence
  ASO H15-4
  misc_feature (1)..(3): 2′-O-(2-Methoxyethyl)-oligori-bonucleotides
  misc_feature (4)..(13): phosphorothioate bonds
  misc_feature (4)..(4): 5′methyl
  misc_feature (10)..(11): 5′methyl
  misc_feature (13)..(13): 5′methyl
  misc_feature (13)..(15): 2′-O-(2-Methoxyethyl)-oligori-bonucleotides
ggucagaagcctcga

SEQ ID NO: 78 - DNA, 16 bp, Artificial Sequence
  ASO H22
  misc_feature (1)..(3): locked nucleic acid
  misc_feature (2)..(2): 5′methyl
  misc_feature (4)..(13): phosphorothioate bonds
  misc_feature (4)..(4): 5′methyl
  misc_feature (6)..(6): 5′methyl
  misc_feature (8)..(8): 5′methyl
  misc_feature (11)..(11): 5′methyl
  misc_feature (14)..(14): 5′methyl
  misc_feature (14)..(16): locked nucleic acid
gcacgctcagcatctg

SEQ ID NO: 79 - DNA, 19 bp, Artificial Sequence
  ASO H23
  misc_feature (1)..(5): locked nucleic acid
  misc_feature (1)..(1): 5′methyl
  misc_feature (3)..(3): 5′methyl
  misc_feature (5)..(13): phosphorothioate bonds
  misc_feature (6)..(6): 5′methyl
  misc_feature (8)..(8): 5′methyl
  misc_feature (10)..(10): 5′methyl
  misc_feature (12)..(12): 5′methyl
  misc_feature (15)..(15): 5′methyl
  misc_feature (15)..(19): locked nucleic acid
  misc_feature (18)..(18): 5′methyl
ctctgcacgctcagcatct

SEQ ID NO: 80 - DNA, 15 bp, Artificial Sequence
  ASO H24
  misc_feature (1)..(3): locked nucleic acid
  misc_feature (2)..(2): 5′methyl
  misc_feature (4)..(12): phosphorothioate bonds
  misc_feature (4)..(4): 5′methyl
  misc_feature (6)..(6): 5′methyl
  misc_feature (8)..(8): 5′methyl
  misc_feature (11)..(11): 5′methyl
  misc_feature (13)..(15): locked nucleic acid
  misc_feature (14)..(14): 5′methyl
gcacgctcagcatct

SEQ ID NO: 81 - DNA, 17 bp, Artificial Sequence
  ASO H25
  misc_feature (1)..(4): locked nucleic acid
  misc_feature (2)..(2): 5′methyl
  misc_feature (5)..(14): phosphorothioate bonds
  misc_feature (5)..(5): 5′methyl
  misc_feature (7)..(7): 5′methyl
  misc_feature (9)..(9): 5′methyl
  misc_feature (14)..(14): 5′methyl
  misc_feature (15)..(17): locked nucleic acid
  misc_feature (17)..(17): 5′methyl
tctgcacgctcagcatc

SEQ ID NO: 82 - DNA, 16 bp, Artificial Sequence
  ASO H26
  misc_feature (1)..(3): locked nucleic acid
  misc_feature (1)..(3): phosphorothioate bonds
  misc_feature (1)..(1): 5′methyl
  misc_feature (4)..(12): phosphorothioate bonds
  misc_feature (4)..(4): 5′methyl
  misc_feature (6)..(6): 5′methyl
  misc_feature (8)..(8): 5′methyl
  misc_feature (10)..(10): 5′methyl
  misc_feature (13)..(13): 5′methyl
  misc_feature (13)..(16): locked nucleic acid
  misc_feature (16)..(16): 5′methyl
ctgcacgctcagcatc

SEQ ID NO: 83 - DNA, 15 bp, Artificial Sequence
  ASO H27
  misc_feature (1)..(3): locked nucleic acid
  misc_feature (4)..(12): phosphorothioate bonds
  misc_feature (4)..(4): 5′methyl
  misc_feature (10)..(11): 5′methyl
  misc_feature (13)..(13): 5′methyl
  misc_feature (13)..(15): locked nucleic acid
ggtcagaagcctcga

SEQ ID NO: 84 - DNA, 15 bp, Artificial Sequence
  ASO H28
  misc_feature (1)..(4): locked nucleic acid
  misc_feature (4)..(4): 5′methyl
  misc_feature (5)..(12): phosphorothioate bonds
  misc_feature (10)..(11): 5′methyl
  misc_feature (13)..(13): 5′methyl
  misc_feature (13)..(15): locked nucleic acid
ggtcagaagcctcga

SEQ ID NO: 85 - DNA, 16 bp, Artificial Sequence
  ASO H29
  misc_feature (1)..(3): locked nucleic acid
  misc_feature (4)..(13): phosphorothioate bonds
  misc_feature (4)..(4): 5′methyl
  misc_feature (8)..(8): 5′methyl
  misc_feature (14)..(16): locked nucleic acid
ggacttgcaggtgtag

SEQ ID NO: 86 - DNA, 17 bp, Artificial Sequence
  ASO H30
  misc_feature (1)..(4): 2′-O-(2-Methoxyethyl)-oligori-bonucleotides
  misc_feature (5)..(12): phosphorothioate bonds
  misc_feature (5)..(5): 5′methyl
  misc_feature (9)..(9): 5′methyl
  misc_feature (14)..(17): 2′-O-(2-Methoxyethyl)-oligori-bonucleotides
aggacttgcaggtguag

SEQ ID NO: 87 - DNA, 16 bp, Artificial Sequence
  ASO H31
  misc_feature (1)..(1): locked nucleic acid
  misc_feature (2)..(2): 2′-O-(2-Methoxyethyl)-oligori-bonucleotides
  misc_feature (3)..(3): locked nucleic acid
  misc_feature (4)..(4): 2′-O-(2-Methoxyethyl)-oligori-bonucleotides
  misc_feature (5)..(12): phosphorothioate bonds
  misc_feature (5)..(5): 5′methyl
  misc_feature (9)..(9): 5′methyl
  misc_feature (13)..(13): 2′-O-(2-Methoxyethyl)-oligori-bonucleotides
  misc_feature (14)..(14): locked nucleic acid
  misc_feature (15)..(15): 2′-O-(2-Methoxyethyl)-oligori-bonucleotides
  misc_feature (16)..(16): locked nucleic acid
aggacttgcaggugua

SEQ ID NO: 88 - DNA, 15 bp, Artificial Sequence
  ASO H32
  misc_feature (1)..(3): 2′-O-(2-Methoxyethyl)-oligori-bonucleotides
  misc_feature (4)..(12): phosphorothioate bonds
  misc_feature (4)..(4): 5′methyl
  misc_feature (8)..(8): 5′methyl
  misc_feature (12)..(12): 5′methyl
  misc_feature (13)..(15): 2′-O-(2-Methoxyethyl)-oligori-bonucleotides
ggacttgcaggtgua

SEQ ID NO: 89 - DNA, 15 bp, Artificial Sequence
  ASO H33
  misc_feature (1)..(3): 2′-O-(2-Methoxyethyl)-oligori-bonucleotides
  misc_feature (4)..(12): phosphorothioate bonds
  misc_feature (5)..(5): 5′methyl
  misc_feature (9)..(9): 5′methyl
  misc_feature (13)..(15): 2′-O-(2-Methoxyethyl)-oligori-bonucleotides
aggacttgcaggugu

SEQ ID NO: 90 - DNA, 16 bp, Artificial Sequence
  ASO H34
  misc_feature (1)..(3): locked nucleic acid
  misc_feature (4)..(13): phosphorothioate bonds
  misc_feature (4)..(4): 5′methyl
  misc_feature (10)..(10): 5′methyl
  misc_feature (12)..(12): 5′methyl
  misc_feature (14)..(16): locked nucleic acid
gaactgcagctcggag

SEQ ID NO: 91 - DNA, 15 bp, Artificial Sequence
  ASO H35
  misc_feature (1)..(3): locked nucleic acid
  misc_feature (4)..(12): phosphorothioate bonds
  misc_feature (4)..(4): 5′methyl
  misc_feature (7)..(7): 5′methyl
  misc_feature (10)..(10): 5′methyl
  misc_feature (12)..(12): 5′methyl
  misc_feature (14)..(16): locked nucleic acid
gaactgcagctcgga

SEQ ID NO: 92 - DNA, 15 bp, Artificial Sequence
  ASO H36
  misc_feature (1)..(3): locked nucleic acid
  misc_feature (4)..(12): phosphorothioate bonds
  misc_feature (12)..(12): 5′methyl
  misc_feature (13)..(15): locked nucleic acid
  misc_feature (15)..(15): 5′methyl
aaggtgaactgcagc

SEQ ID NO: 93 - DNA, 20 bp, Artificial Sequence
  ASO 1477-1
  misc_feature (1)..(5): 2′-O-(2-Methoxyethyl)-oligori-bonucleotides
  misc_feature (2)..(2): 5′methyl
  misc_feature (4)..(4): 5′methyl
  misc_feature (6)..(15): phosphorothioate bonds
  misc_feature (7)..(7): 5′methyl
  misc_feature (14)..(14): 5′methyl
  misc_feature (16)..(20): 2′-O-(2-Methoxyethyl)-oligori-bonucleotides
  misc_feature (20)..(20): 5′methyl
gcgcatcaaaggtcagaagc

SEQ ID NO: 94 - DNA, 20 bp, Artificial Sequence
  ASO 1659-2
  misc_feature (1)..(5): 2′-O-(2-Methoxyethyl)-oligori-bonucleotides
  misc_feature (1)..(2): 5′Methyl
  misc_feature (6)..(15): phosphorothioate bonds
  misc_feature (12)..(12): 5′Methyl
  misc_feature (16)..(20): 2′-O-(2-Methoxyethyl)-oligori-bonucleotides
ccugggaatgtcaagguugg

SEQ ID NO: 95 - DNA, 17 bp, Artificial Sequence
  ASO 6-1
  misc_feature (1)..(16): phosphorothioate bonds
  misc_feature (1)..(4): 2′-O-(2-Methoxyethyl)-oligori-bonucleotides
  misc_feature (2)..(2): 5′methyl
  misc_feature (4)..(4): 5′methyl
  misc_feature (7)..(7): 5′methyl
  misc_feature (10)..(11): 5′methyl
  misc_feature (13)..(14): 5′methyl
  misc_feature (14)..(17): 2′-O-(2-Methoxyethyl)-oligori-bonucleotides
  misc_feature (17)..(17): 5′methyl
gcucgtctacctccuuc

SEQ ID NO: 96 - DNA, 17 bp, Artificial Sequence
  ASO 6-2
  misc_feature (1)..(2): phosphorothioate bonds
  misc_feature (1)..(4): 2′-O-(2-Methoxyethyl)-oligori-bonucleotides
  misc_feature (2)..(2): 5′methyl
  misc_feature (4)..(14): phosphorothioate bonds
  misc_feature (4)..(4): 5′methyl
  misc_feature (7)..(7): 5′methyl
  misc_feature (10)..(11): 5′methyl
  misc_feature (13)..(14): 5′methyl
  misc_feature (14)..(17): 2′-O-(2-Methoxyethyl)-oligori-bonucleotides
  misc_feature (16)..(17): phosphorothioate bonds
  misc_feature (17)..(17): 5′methyl
gcucgtctacctccuuc

SEQ ID NO: 97 - DNA, 19 bp, Artificial Sequence
  ASO 6-3
  misc_feature (1)..(18): phosphorothioate bonds
  misc_feature (1)..(5): 2′-O-(2-Methoxyethyl)-oligori-bonucleotides
  misc_feature (3)..(3): 5′methyl
  misc_feature (5)..(5): 5′methyl
  misc_feature (8)..(8): 5′methyl
  misc_feature (11)..(12): 5′methyl
  misc_feature (14)..(15): 5′methyl
  misc_feature (15)..(19): 2′-O-(2-Methoxyethyl)-oligori-bonucleotides
  misc_feature (18)..(18): 5′methyl
ugcucgtctacctccuuca

SEQ ID NO: 98 - DNA, 19 bp, Artificial Sequence
  ASO 6-4
  misc_feature (1)..(2): phosphorothioate bonds
  misc_feature (1)..(5): 2′-O-(2-Methoxyethyl)-oligori-bonucleotides
  misc_feature (3)..(3): 5′methyl
  misc_feature (5)..(15): phosphorothioate bonds
  misc_feature (5)..(5): 5′methyl
  misc_feature (8)..(8): 5′methyl
  misc_feature (11)..(12): 5′methyl
  misc_feature (14)..(15): 5′methyl
  misc_feature (15)..(19): 2′-O-(2-Methoxyethyl)-oligori-bonucleotides
  misc_feature (18)..(19): phosphorothioate bonds
  misc_feature (18)..(18): 5′methyl
ugcucgtctacctccuuca

SEQ ID NO: 99 - DNA, 17 bp, Artificial Sequence
  ASO 6-5
  misc_feature (1)..(4): 2′-O-(2-Methoxyethyl)-oligori-bonucleotides
  misc_feature (2)..(2): 5′methyl
  misc_feature (4)..(14): phosphorothioate bonds
  misc_feature (4)..(4): 5′methyl
  misc_feature (7)..(7): 5′methyl
  misc_feature (10)..(11): 5′methyl
  misc_feature (13)..(14): 5′methyl
  misc_feature (14)..(17): 2′-O-(2-Methoxyethyl)-oligori-bonucleotides
  misc_feature (17)..(17): 5′methyl
gcucgtctacctccuuc

SEQ ID NO: 100 - DNA, 16 bp, Artificial Sequence
  ASO 7-1
  misc_feature (1)..(16): phosphorothioate bonds
  misc_feature (1)..(3): 2′-O-(2-Methoxyethyl)-oligori-bonucleotides
  misc_feature (2)..(2): 5′methyl
  misc_feature (4)..(4): 5′methyl
  misc_feature (7)..(7): 5′methyl
  misc_feature (10)..(11): 5′methyl
  misc_feature (13)..(14): 5′methyl
  misc_feature (14)..(16): 2′-O-(2-Methoxyethyl)-oligori-bonucleotides
gcucgtctacctccuu

SEQ ID NO: 101 - DNA, 16 bp, Artificial Sequence
  ASO 7-2
  misc_feature (1)..(16): phosphorothioate bonds
  misc_feature (1)..(1): 2′-O-(2-Methoxyethyl)-oligori-bonucleotides
  misc_feature (2)..(2): 5′methyl
  misc_feature (2)..(2): locked nucleic acid
  misc_feature (3)..(3): 2′-O-(2-Methoxyethyl)-oligori-bonucleotides
  misc_feature (4)..(4): 5′methyl
  misc_feature (7)..(7): 5′methyl
  misc_feature (10)..(11): 5′methyl
  misc_feature (13)..(14): 5′methyl
  misc_feature (14)..(14): 2′-O-(2-Methoxyethyl)-oligori-bonucleotides
  misc_feature (15)..(15): locked nucleic acid
  misc_feature (16)..(16): 2′-O-(2-Methoxyethyl)-oligori-bonucleotides
gcucgtctacctccuu

SEQ ID NO: 102 - DNA, 18 bp, Artificial Sequence
  ASO 7-3
  misc_feature (1)..(18): phosphorothioate bonds
  misc_feature (1)..(4): 2′-O-(2-Methoxyethyl)-oligori-bonucleotides
  misc_feature (3)..(3): 5′methyl
  misc_feature (5)..(5): 5′methyl
  misc_feature (8)..(8): 5′methyl
  misc_feature (11)..(12): 5′methyl
  misc_feature (14)..(15): 5′methyl
  misc_feature (15)..(18): 2′-O-(2-Methoxyethyl)-oligori-bonucleotides
  misc_feature (18)..(18): 5′methyl
ugcucgtctacctccuuc

SEQ ID NO: 103 - DNA, 18 bp, Artificial Sequence
  ASO 7-4
  misc_feature (1)..(2): phosphorothioate bonds
  misc_feature (1)..(4): 2′-O-(2-Methoxyethyl)-oligori-bonucleotides
  misc_feature (3)..(3): 5′methyl
  misc_feature (4)..(15): phosphorothioate bonds
  misc_feature (5)..(5): 5′methyl
  misc_feature (8)..(8): 5′methyl
  misc_feature (11)..(12): 5′methyl
  misc_feature (14)..(15): 5′methyl
  misc_feature (15)..(18): 2′-O-(2-Methoxyethyl)-oligori-bonucleotides
  misc_feature (18)..(19): phosphorothioate bonds
  misc_feature (18)..(18): 5′methyl
ugcucgtctacctccuuc

SEQ ID NO: 104 - DNA, 16 bp, Artificial Sequence
  ASO 7-5
  misc_feature (1)..(3): 2′-O-(2-Methoxyethyl)-oligori-bonucleotides
  misc_feature (2)..(2): 5′methyl
  misc_feature (4)..(14): phosphorothioate bonds
  misc_feature (4)..(4): 5′methyl
  misc_feature (7)..(7): 5′methyl
  misc_feature (10)..(11): 5′methyl
  misc_feature (13)..(14): 5′methyl
  misc_feature (14)..(16): 2′-O-(2-Methoxyethyl)-oligori-bonucleotides
gcucgtctacctccuu

SEQ ID NO: 105 - DNA, 18 bp, Artificial Sequence
  ASO 7-6
  misc_feature (1)..(4): 2′-O-(2-Methoxyethyl)-oligori-bonucleotides
  misc_feature (3)..(3): 5′methyl
  misc_feature (5)..(15): phosphorothioate bonds
  misc_feature (5)..(5): 5′methyl
  misc_feature (8)..(8): 5′methyl
  misc_feature (11)..(12): 5′methyl
  misc_feature (14)..(15): 5′methyl
  misc_feature (15)..(18): 2′-O-(2-Methoxyethyl)-oligori-bonucleotides
  misc_feature (18)..(18): 5′methyl
ugcucgtctacctccuuc

SEQ ID NO: 106 - DNA, 16 bp, Artificial Sequence
  ASO 8-1
  misc_feature (1)..(16): phosphorothioate bonds
  misc_feature (1)..(3): 2′-O-(2-Methoxyethyl)-oligori-bonucleotides
  misc_feature (3)..(3): 5′methyl
  misc_feature (5)..(5): 5′methyl
  misc_feature (8)..(8): 5′methyl
  misc_feature (11)..(12): 5′methyl
  misc_feature (14)..(15): 5′methyl
  misc_feature (14)..(16): 2′-O-(2-Methoxyethyl)-oligori-bonucleotides
ugctcgtctacctccu

SEQ ID NO: 107 - DNA, 16 bp, Artificial Sequence
  ASO 8-2
  misc_feature (1)..(2): phosphorothioate bonds
  misc_feature (1)..(1): 2′-O-(2-Methoxyethyl)-oligori-bonucleotides
  misc_feature (2)..(2): locked nucleic acid
  misc_feature (3)..(3): 5′methyl
  misc_feature (3)..(14): phosphorothioate bonds
  misc_feature (3)..(3): 2′-O-(2-Methoxyethyl)-oligori-bonucleotides
  misc_feature (5)..(5): 5′methyl
  misc_feature (8)..(8): 5′methyl
  misc_feature (11)..(12): 5′methyl
  misc_feature (14)..(15): 5′methyl
  misc_feature (14)..(14): 2′-O-(2-Methoxyethyl)-oligori-bonucleotides
  misc_feature (15)..(16): phosphorothioate bonds
  misc_feature (15)..(15): locked nucleic acid
  misc_feature (16)..(16): 2′-O-(2-Methoxyethyl)-oligori-bonucleotides
ugctcgtctacctccu

SEQ ID NO: 108 - DNA, 20 bp, Artificial Sequence
  ASO 8-3
  misc_feature (1)..(20): phosphorothioate bonds
  misc_feature (1)..(5): 2′-O-(2-Methoxyethyl)-oligori-bonucleotides
  misc_feature (2)..(2): 5′methyl
  misc_feature (5)..(5): 5′methyl
  misc_feature (7)..(7): 5′methyl
  misc_feature (10)..(10): 5′methyl
  misc_feature (13)..(14): 5′methyl
  misc_feature (16)..(17): 5′methyl
  misc_feature (16)..(20): 2′-O-(2-Methoxyethyl)-oligori-bonucleotides
  misc_feature (20)..(20): 5′methyl
ucugctcgtctacctccuuc

SEQ ID NO: 109 - DNA, 20 bp, Artificial Sequence
  ASO 8-4
  misc_feature (1)..(2): phosphorothioate bonds
  misc_feature (1)..(5): 2′-O-(2-Methoxyethyl)-oligori-bonucleotides
  misc_feature (2)..(2): 5′methyl
  misc_feature (5)..(16): phosphorothioate bonds
  misc_feature (5)..(5): 5′methyl
  misc_feature (7)..(7): 5′methyl
  misc_feature (10)..(10): 5′methyl
  misc_feature (13)..(14): 5′methyl
  misc_feature (16)..(17): 5′methyl
  misc_feature (16)..(20): 2′-O-(2-Methoxyethyl)-oligori-bonucleotides
  misc_feature (19)..(20): phosphorothioate bonds
  misc_feature (20)..(20): 5′methyl
ucugctcgtctacctccuuc

SEQ ID NO: 110 - DNA, 16 bp, Artificial Sequence
  ASO 8-5
  misc_feature (1)..(3): 2′-O-(2-Methoxyethyl)-oligori-bonucleotides
  misc_feature (3)..(3): 5′methyl
  misc_feature (4)..(14): phosphorothioate bonds
  misc_feature (5)..(5): 5′methyl
  misc_feature (8)..(8): 5′methyl
  misc_feature (11)..(12): 5′methyl
  misc_feature (14)..(15): 5′methyl
  misc_feature (14)..(16): 2′-O-(2-Methoxyethyl)-oligori-bonucleotides
ugctcgtctacctccu

SEQ ID NO: 111 - DNA, 15 bp, Artificial Sequence
  ASO 18-1
  misc_feature (1)..(15): phosphorothioate bonds
  misc_feature (1)..(3): 2′-O-(2-Methoxyethyl)-oligori-bonucleotides
  misc_feature (3)..(3): 5′methyl
  misc_feature (5)..(5): 5′methyl
  misc_feature (8)..(8): 5′methyl
  misc_feature (11)..(12): 5′methyl
  misc_feature (13)..(15): 2′-O-(2-Methoxyethyl)-oligori-bonucleotides
  misc_feature (14)..(15): 5′methyl
ugctcgtctaccucc

SEQ ID NO: 112 - DNA, 15 bp, Artificial Sequence
  ASO 18-2
  misc_feature (1)..(15): phosphorothioate bonds
  misc_feature (1)..(3): locked nucleic acid
  misc_feature (3)..(3): 5′methyl
  misc_feature (5)..(5): 5′methyl
  misc_feature (8)..(8): 5′methyl
  misc_feature (11)..(12): 5′methyl
  misc_feature (13)..(15): locked nucleic acid
  misc_feature (14)..(15): 5′methyl
ugctcgtctaccucc

SEQ ID NO: 113 - DNA, 17 bp, Artificial Sequence
  ASO 18-3
  misc_feature (1)..(1): 5′methyl
  misc_feature (1)..(17): phosphorothioate bonds
  misc_feature (1)..(4): 2′-O-(2-Methoxyethyl)-oligori-bonucleotides
  misc_feature (4)..(4): 5′methyl
  misc_feature (6)..(6): 5′methyl
  misc_feature (9)..(9): 5′methyl
  misc_feature (12)..(13): 5′methyl
  misc_feature (14)..(17): 2′-O-(2-Methoxyethyl)-oligori-bonucleotides
  misc_feature (15)..(16): 5′methyl
cugctcgtctaccuccu

SEQ ID NO: 114 - DNA, 15 bp, Artificial Sequence
  ASO 18-5
  misc_feature (1)..(3): 2′-O-(2-Methoxyethyl)-oligori-bonucleotides
  misc_feature (3)..(3): 5′methyl
  misc_feature (4)..(13): phosphorothioate bonds
  misc_feature (5)..(5): 5′methyl
  misc_feature (8)..(8): 5′methyl
  misc_feature (11)..(12): 5′methyl
  misc_feature (13)..(15): 2′-O-(2-Methoxyethyl)-oligori-bonucleotides
  misc_feature (14)..(15): 5′methyl
ugctcgtctaccucc